(19) United States
(12) Patent Application Publication
(10) Pub. No.: US 2025/0364084 A1
(43) Pub. Date: Nov. 27, 2025

(54) METHODS FOR USING A MACHINE LEARNING ALGORITHM FOR OMIC ANALYSIS

(71) Applicant: Seer, Inc., Redwood City, CA (US)

(72) Inventors: Daniel HORNBURG, Foster City, CA (US); Harendra GUTURU, Oakland, CA (US); Moaraj HASAN, San Francisco, CA (US); Shadi ROSHDIFERDOSI, San Carlos, CA (US); Amir ALAVI, San Francisco, CA (US); Tristan BROWN, Sunnyvale, CA (US); Jian WANG, San Diego, CA (US); Alexey STUKALOV, Redwood City, CA (US)

(21) Appl. No.: 19/104,122

(22) PCT Filed: Aug. 17, 2023
(86) PCT No.: PCT/US2023/072417

§ 371 Date: Feb. 14, 2025

Related U.S. Application Data
(60) Provisional application No. 63/399,205, filed on Aug. 18, 2022.
(60) Provisional application No. 63/373,700, filed on Aug. 26, 2022.

(51) Int. Cl. G16B 40/10; G16C 20/20

(52) U.S. Cl. CPC G16B 40/10; G16C 20/20

(57) ABSTRACT

In some aspects, the present disclosure provides a computer-implemented method for quantifying a molecule using a machine learning algorithm. The computer-implemented method can comprise providing an input dataset comprising one or more features representing a quantity of the molecule measured using at least a first condition. The computer-implemented method can comprise processing the input dataset, using a machine learning algorithm, to generate an adjusted quantity of the molecule at a second condition.

### CROSS-REFERENCE

This application claims the benefit of U.S. Provisional Application No. 63/399,205, filed Aug. 18, 2022, and U.S. Provisional Application No. 63/373,700, filed Aug. 26, 2022, each of which is incorporated herein by reference in its entirety.

### BACKGROUND

Blood plasma is an ideal biospecimen to assess human health and disease states because it connects to almost all tissues and is accessible longitudinally and with minimal invasiveness. However, the wide dynamic range of the plasma proteome, over 10 orders of magnitude, perhaps millions of proteoforms, creates challenges for standard proteomic approaches and prevents wide-spread adoption of untargeted deep proteomics at scale.

### SUMMARY

In some aspects, provided herein are computer-implemented systems and methods for predicting concentrations of molecules in an original sample using quantities of molecules measured from various readout technologies (e.g., mass spectrometry, sequencing, ELISA assays, etc.) and a machine learning algorithm. In some aspects, provided herein are computer-implemented systems and methods for training a machine learning algorithm for predicting compositions of original samples. In some embodiments, the machine learning algorithm may use data representative of derivatives (or relative or absolute changes) in the quantity of a molecule with respect to a change in an experimental parameter used for obtaining the quantity. The derivative (or change) information can provide thermodynamic and dynamic information that can tie different molecules or complexes of molecules (e.g., having different chemical structures) subjected to similar deviations and errors in measurement.

An aspect of the present disclosure provides a computer-implemented method for training a machine learning algorithm for molecule quantification comprising: providing an input dataset comprising one or more features that represent changes in quantities for a plurality of molecules with respect to one or more physicochemical parameters, wherein the changes are measured using at least a first condition; processing, using the machine learning algorithm, the input dataset to generate an output value; and adjusting one or more numerical parameters of the machine learning algorithm based on a loss function based at least in part on the output value, such that the output value accounts for a difference between (i) the quantities for at least a portion of the plurality of molecules and (ii) reference quantities for a plurality of reference molecules, wherein the reference quantities are measured using at least a second condition.

In some embodiments, the first condition comprises binding the plurality of molecules to a surface. In some embodiments, the surface comprises a sensor element surface. In some embodiments, the sensor element surface comprises a particle surface. In some embodiments, the particle surface is a nanoparticle surface. In some embodiments, the particle surface is a microparticle surface. In some embodiments, the particle surface comprises pores. In some embodiments, the binding is via adsorption. In some embodiments, the binding is non-specific. In some embodiments, the binding is specific. In some embodiments, the plurality of molecules forms a corona on the particle surface. In some embodiments, the quantities comprise measured intensities. In some embodiments, the measured intensities comprise mass spectrometry (MS) intensities. In some embodiments, the MS intensities comprise peptide intensities, protein group intensities, peptide group intensities, or combinations thereof. In some embodiments, the plurality of molecules comprises a plurality of proteins. In some embodiments, the input dataset comprises measured intensities of a plurality of peptides, wherein the plurality of peptides is derived from the plurality of proteins. In some embodiments, the MS intensities comprise small molecule intensities. In some embodiments, the MS intensities are based on data-independent acquisition (DIA) MS, data-dependent acquisition (DDA) MS, or both. In some embodiments, the MS intensities are based on liquid-chromatography tandem mass spectrometry (LC-MS/MS). In some embodiments, the measured intensities comprise the fluorescence signals. In some embodiments, the measured intensities comprise an induced current. In some embodiments, the measured intensities are obtained using a nanopore sensor. In some embodiments, the measured intensities are obtained using an immunoassay. In some embodiments, the quantities are determined using a nucleic acid sequencer. In some embodiments, the reference quantities comprise measured intensities. In some embodiments, the measured intensities comprise mass spectrometry (MS) intensities. In some embodiments, the MS intensities comprise peptide intensities, protein group intensities, or both. In some embodiments, the MS intensities comprise small molecule intensities. In some embodiments, the MS intensities are based on data-independent acquisition (DIA) MS, data-dependent acquisition (DDA) MS, or both. In some embodiments, the MS intensities are based on liquid-chromatography tandem mass spectrometry (LC-MS/MS). In some embodiments, the measured intensities comprise the fluorescence signals. In some embodiments, the measured intensities comprise an induced current. In some embodiments, the measured intensities are obtained using a nanopore sensor. In some embodiments, the measured intensities are obtained using an immunoassay. In some embodiments, the quantities are determined using a nucleic acid sequencer. In some embodiments, the one or more physicochemical parameters comprise: sample to surface ratio, incubation time, pH, salt concentration, ionic strength, solvent composition, solvent dielectric constant, crowding agent concentration, temperature, sample composition, surfactant concentration, concentration of enzymes, activity of enzymes, chemical reactions, concentrations of small molecules, surface chemistry, or any combination thereof. In some embodiments, the sample to surface ratio comprises (i) volume of sample to surface area of the surface, (ii) volume of sample to mass of a substrate comprising the surface, (iii) mass of sample to surface area of the surface, or (iv) mass of sample to mass of the substrate comprising the surface. In some embodiments, the one or more physicochemical parameters comprise a ratio of surface area of the surface to a volume of a sample comprising the plurality of molecules. In some embodiments, the one or more physicochemical parameters comprise a ratio of surface area of the surface to a concentration of the plurality of molecules in a sample. In some embodiments, the one or more physicochemical parameters comprise a ratio of surface area of the surface to a mass of the plurality of molecules in a sample. In some embodiments, the one or more physicochemical parameters comprise a ratio of mass of a substrate comprising the surface to a volume of a sample comprising the plurality of molecules. In some embodiments, the one or more physicochemical parameters comprise a ratio of mass of a substrate comprising the surface to a concentration of the plurality of molecules in a sample. In some embodiments, the one or more physicochemical parameters comprise a ratio of mass of a substrate comprising the surface to a mass of the plurality of molecules in a sample. In some embodiments, the one or more physicochemical parameters comprise surface chemistry. In some embodiments, the one or more physicochemical parameters comprise an incubation time for the plurality of molecules to the surface. In some embodiments, the one or more features represent changes in quantities for the plurality of molecules with respect incubation time when the incubation time is at least 1, 15, 30, or 60 seconds. In some embodiments, the one or more features represent changes in quantities for the plurality of molecules with respect incubation time when the incubation time is at least 1, 15, 30, or 60 minutes. In some embodiments, the one or more features represent changes in quantities for the plurality of molecules with respect incubation time when the incubation time is at least 1, 2, 3, 4, 8, 12, 16, 20, or 24 hours. In some embodiments, the one or more features represent changes in quantities for the plurality of molecules with respect incubation time when the incubation time is at least 1, 2, 3, 4, 5, 6 or 7 days. In some embodiments, the one or more features represent changes in quantities for the plurality of molecules with respect incubation time when the incubation time is at most 1, 2, 3, 4, 5, 6 or 7 days. In some embodiments, the one or more features represent changes in quantities for the plurality of molecules with respect incubation time when the incubation time is at most 1, 2, 3, 4, 8, 12, 16, 20, or 24 hours. In some embodiments, the one or more features represent changes in quantities for the plurality of molecules with respect incubation time when the incubation time is at most 1, 15, 30, or 60 minutes. In some embodiments, the one or more features represent changes in quantities for the plurality of molecules with respect to incubation time when the incubation time is at most 1, 15, 30, or 60 seconds. In some embodiments, the input dataset comprises a first plurality of quantities measured at the first condition. In some embodiments, the input dataset comprises a second plurality of quantities measured at the second condition. In some embodiments, the plurality of molecules comprise at least 20, 40, 60, 80, 100, 200, 300, 400, 500, 600, 700, 800, 900, 1000, 2000, 3000, 4000, 5000, 6000, 7000, 8000, 9000, 10000, 11000, 12000, 13000, 14000, 15000, 16000, 17000, 18000, 19000, or 20000 molecules. In some embodiments, the plurality of molecules comprise at most 20, 40, 60, 80, 100, 200, 300, 400, 500, 600, 700, 800, 900, 1000, 2000, 3000, 4000, 5000, 6000, 7000, 8000, 9000, 10000, 11000, 12000, 13000, 14000, 15000, 16000, 17000, 18000, 19000, or 20000 molecules. In some embodiments, the one or more features comprise at least 2, 3, 4, 5, 6, 7, 8, 9, 10, 20, 30, 40, 50, 60, 70, 80, 90, or 100 features for each molecule in the plurality of molecules. In some embodiments, the one or more features comprise at most 2, 3, 4, 5, 6, 7, 8, 9, 10, 20, 30, 40, 50, 60, 70, 80, 90, or 100 features for each molecule in the plurality of molecules. In some embodiments, the one or more physicochemical parameters comprise at least 2, 3, 4, 5, 6, 7, 8, 9, 10, 20, 30, 40, 50, 60, 70, 80, 90, or 100 physicochemical parameters. In some embodiments, the one or more physicochemical parameters comprise at most 2, 3, 4, 5, 6, 7, 8, 9, 10, 20, 30, 40, 50, 60, 70, 80, 90, or 100 physicochemical parameters. In some embodiments, the output value is a normalization value for adjusting the quantities of the plurality of molecules using the first condition to predicted quantities of the molecules using the second condition. In some embodiments, the normalization value is the difference between a quantity and a reference quantity. In some embodiments, the normalization value is a ratio between a quantity and a reference quantity. In some embodiments, the output value is a reference quantity. In some embodiments, the molecule is not in the input dataset. In some embodiments, a magnitude of the predicted quantity is below a detection limit of the method or device used to generate the machine learning model. In some embodiments, a magnitude of the predicted quantity is below a detection limit of the method or device used to generate the input dataset. In some embodiments, a first scale of the predicted quantity is different from a second scale of the method or device used to generate the machine learning model. In some embodiments, the second scale of the method or device comprises a deviation. In some embodiments, the method or device comprises MS, and wherein the deviation is based on a number of charges and flyability. In some embodiments, the output value is a corrected or tuned quantity. In some embodiments, the corrected quantity is a corrected MS intensity. In some embodiments, the adjusting comprises at least partially optimizing a mean squared error loss function when the input dataset comprises a quantity in the quantities and a reference quantity in the reference quantities. In some embodiments, the adjusting comprises at least partially optimizing a logistic loss function when the input dataset does not comprise either a quantity in the quantities or a reference quantity in the reference quantities. In some embodiments, the computer-implemented further comprises receiving a second input dataset comprising: (a) a second set of features that represent a second set of changes in a second set of quantities for a second plurality of molecules with respect to the one or more physicochemical parameters, wherein the second set of changes are measured using at least a third condition; (b) processing, using the machine learning algorithm, the second input dataset to generate a second output value; and adjusting the one or more numerical parameters of the machine learning algorithm based on a second loss function based at least in part on the second output value. In some embodiments, the second plurality of molecules comprises no molecules in common with the plurality of molecules. In some embodiments, the second plurality of molecules comprises one or more molecules in common with the plurality of molecules. In some embodiments, the second plurality of molecules comprises one or more molecules not in the plurality of molecules. In some embodiments, the second input dataset comprises the reference quantities. In some embodiments, the second input dataset comprises a plurality of differences between the quantities and the reference quantities. In some embodiments, a reference quantity of a reference molecule in the reference molecules and a quantity of a molecule in the molecules have a similar change with respect to the one or more physicochemical parameters. In some embodiments, the reference molecules are the same as the at least the portion of the molecules. In some embodiments, the reference quantities of the reference molecules are derived from the same sample as the at least the portion of the molecules. In some embodiments, the reference quantities comprise average abundance values of the molecules over a plurality of samples. In some embodiments, the average abundance values are concentration values, intensities values, or relative abundance values. In some embodiments, the second condition comprises a neat measurement condition. In some embodiments, the neat measurement condition does not comprise binding the molecule to the surface. In some embodiments, the reference quantities comprise an aggregate of measurements of samples. In some embodiments, the reference quantity of a reference molecule in the second input dataset is based on a reference signal of another molecule. In some embodiments, the second condition comprises using liquid chromatography with a gradient length equal to or greater than 30 minutes or 2 hours. In some embodiments, the second condition comprises gas phase separation. In some embodiments, the second condition comprises a different ratio of surface area of the surface to a volume of a sample comprising the biomolecule compared to the first condition. In some embodiments, the second condition comprises a different ratio of a surface area of the surface to a concentration of the biomolecule in a sample compared to the first condition. In some embodiments, the second condition comprises a different ratio of a surface area of the surface to a mass of the biomolecule in a sample compared to the first condition. In some embodiments, the second condition comprises a different ratio of a mass of a substrate comprising the surface to a volume of a sample comprising the biomolecule compared to the first condition. In some embodiments, the second condition comprises a different ratio of a mass of a substrate comprising the surface to a concentration of the biomolecule in a sample compared to the first condition. In some embodiments, the second condition comprises a different ratio of a mass of a substrate comprising the surface to a mass of the biomolecule in a sample compared to the first condition. In some embodiments, the second condition comprises a different ratio of the biomolecule to the surface in a sample compared to the first condition. In some embodiments, the second condition comprises the surface with a different surface charge compared to the first condition. In some embodiments, the second condition comprises the surface with a different surface functionalization compared to the first condition. In some embodiments, the second condition comprises a different incubation time for binding the biomolecule to the surface compared to the first condition. In some embodiments, the plurality of molecules comprises a plurality of biomolecules. In some embodiments, the plurality of molecules comprises a plurality of proteins. In some embodiments, the plurality of molecules comprises a plurality of proteoforms. In some embodiments, the plurality of proteoforms comprises a splicing variant. In some embodiments, the plurality of proteoforms comprises an allelic variant. In some embodiments, the plurality of proteoforms comprises a post-translational cleavage variant. In some embodiments, the plurality of proteoforms comprises a phosphorylated variant. In some embodiments, the plurality of molecules comprises a plurality of lipids. In some embodiments, the plurality of molecules comprises a plurality of nucleic acids. In some embodiments, the plurality of molecules comprises a plurality of metabolites. In some embodiments, the plurality of molecules comprises a plurality of secreted molecules. In some embodiments, the first condition, the second condition, or both comprises binding a molecule in the plurality of molecules to an antibody. In some embodiments, the first condition, the second condition, or both comprises binding the molecule to a pair of antibodies. In some embodiments, the pair of antibodies comprises complementary single-stranded nucleic acid sequences attached thereto, such that when the pair of antibodies bind to the molecule, the complementary nucleic acids hybridize to form a double stranded nucleic acid. In some embodiments, the double stranded nucleic acid is configured to form a binding complex with a polymerase and a plurality of nucleotides, nucleosides, nucleotide analogs, and/or nucleoside analogs to perform an amplification reaction to produce a detectable signal. In some embodiments, the first condition, the second condition, or both comprises binding a molecule in the plurality of molecules to an aptamer. In some embodiments, the one or more aptamers are coupled to a surface via a cleavable linker. In some embodiments, the surface is a particle surface. In some embodiments, the cleavable linker is photocleavable. In some embodiments, the first condition, the second condition, or both comprises contacting the molecule and the aptamer with a macromolecular competitor configured to, in a fluid composition, reduce dissociation of a complex comprising the one or more aptamers and the molecule. In some embodiments, the macromolecular competitor is a polyanionic macromolecule. In some embodiments, the first condition, the second condition, or both comprises protein sequencing, and the plurality of molecules comprises a plurality of proteins. In some embodiments, the protein sequencing comprises (i) digesting the plurality of proteins to generate a plurality of protein fragments, (ii) immobilizing the plurality of protein fragments to a semiconductor substrate, (iii) contacting the plurality of protein fragments with a plurality of labeled recognizers, wherein the plurality of labeled recognizers are configured to attach to a predetermined chemical moiety in the plurality of protein fragments at the N-terminus of the plurality of protein fragments, (iv) exciting the plurality of labeled recognizers to detect the plurality of labeled recognizers, thereby detecting the predetermined chemical moiety, (v) removing an amino acid from the N-terminus of the plurality of protein fragments, (vi) contacting the plurality of protein fragments with a second plurality of labeled recognizers, (vii) exciting the second plurality of labeled recognizers to detect a second amino acid from the N-terminus of the plurality of protein fragments, thereby performing the protein sequencing. In some embodiments, the one or more features are obtained from a sample comprising the plurality of molecules. In some embodiments, the sample comprises at most about 1000, 100, 10, 1, 0.1, 0.01, or 0.001 nanograms of biomolecules. In some embodiments, the sample comprises at most about 1000, 100, 10, 1, 0.1, 0.01, or 0.001 nanograms of biomolecules per mL of the sample. In some embodiments, the sample comprises biomolecules from at most about 1000, 100, 10, or 1 cell. In some embodiments, the sample comprises at most about 1000, 100, 10, 1, 0.1, 0.01, or 0.001 microliters. In some embodiments, the sample comprises a complex biological sample. In some embodiments, the sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some embodiments, the biological sample comprises plasma or serum. In some embodiments, the predicted quantities of the plurality of molecules is more accurate than the quantities of the plurality of molecules by at least 1, 2, 3, 4, 5, 6, 7, 8, 9, 10, 15, 20, 25, 30, 35, 40, 45, or 50 percent. In some embodiments, a coefficient of determination between the predicted quantities of the plurality of molecules and the reference quantities of the pluralities of reference molecules is at least 0.7, 0.8, 0.85, 0.9, 0.91, 0.92, 0.93, 0.94, 0.95, 0.96, 0.97, 0.98, or 0.99, when the plurality of molecules and the plurality of reference molecules are the same and the coefficient of determination is measured with a k-fold cross validation, wherein k is an integer greater than 1. In some embodiments, a first coefficient of determination between the predicted quantities of the plurality of molecules and the reference quantities of the pluralities of reference molecules is greater than a second coefficient of determination between the quantities of the plurality of molecules and the reference quantities of the plurality of reference molecules by at least 0.01, 0.02, 0.03, 0.04, 0.05, 0.06, 0.07, 0.08, 0.09, 0.1, 0.2, 0.3, 0.4, or 0.5 when the plurality of molecules and the plurality of reference molecules are the same and the coefficient of determination is measured with a k-fold cross validation, wherein k is an integer greater than 1. In some embodiments, a mean absolute error (MAE) between the predicted quantities of the plurality of molecules and the reference quantities of the pluralities of molecules is at most 1, 2, 3, 4, 5, 6, 7, 8, 9, 10, 15, 20, 25, or 30 percent of the standard deviation of the reference quantities when the plurality of molecules and the plurality of reference molecules are the same and the MAE is measured with a k-fold cross validation, wherein k is an integer greater than 1. In some embodiments, a first mean absolute error (MAE) between the predicted quantities of the plurality of molecules and the reference quantities of the pluralities of molecules is less than a second MAE between the quantities of the plurality of molecules and the reference quantities of the plurality of molecules by at least 1, 2, 3, 4, 5, 6, 7, 8, 9, 10, 15, 20, 25, or 30 percent of the standard deviation of the reference quantities when the plurality of molecules and the plurality of reference molecules are the same and the MAE is measured with a k-fold cross validation, wherein k is an integer greater than 1.

Another aspect of the present disclosure provides a computer-implemented method for quantifying a molecule using a machine learning algorithm, comprising: providing an input dataset comprising one or more features representing a quantity of the molecule measured using at least a first condition; processing the input dataset, using the machine learning algorithm trained according to any one of the methods disclosed herein, to generate an adjusted quantity of the molecule at a second condition. In some embodiments, the input dataset comprises one or more features for a plurality of quantities of a plurality of molecules. In some embodiments, the plurality of quantities comprise at least 20, 40, 60, 80, 100, 200, 300, 400, 500, 600, 700, 800, 900, 1000, 2000, 3000, 4000, 5000, 6000, 7000, 8000, 9000, 10000, or 20000 quantities. In some embodiments, the plurality of quantities comprise at most 20, 40, 60, 80, 100, 200, 300, 400, 500, 600, 700, 800, 900, 1000, 2000, 3000, 4000, 5000, 6000, 7000, 8000, 9000, 10000, or 20000 quantities. In some embodiments, the plurality of molecules comprise at least 20, 40, 60, 80, 100, 200, 300, 400, 500, 600, 700, 800, 900, 1000, 2000, 3000, 4000, 5000, 6000, 7000, 8000, 9000, 10000, or 20000 molecules. In some embodiments, the plurality of molecules comprise at most 20, 40, 60, 80, 100, 200, 300, 400, 500, 600, 700, 800, 900, 1000, 2000, 3000, 4000, 5000, 6000, 7000, 8000, 9000, 10000, or 20000 molecules.

Another aspect of the present disclosure provides a computer-implemented method for training a machine learning algorithm for biomolecule quantification comprising: measuring quantities of a plurality of proteins in a sample, by: (i) contacting the plurality of proteins with a surface to generate a plurality of adsorbed proteins; and (ii) performing mass spectrometry (MS) using the plurality of adsorbed proteins to obtain the quantities, wherein the quantities comprise a deviation or a noise introduced by the contacting in (i); repeating the contacting and performing MS using a set of different experimental conditions to generate a set of quantities, wherein the set of different experimental conditions are different in (i) ratios of the surface to the plurality of proteins, (ii) incubation time used for the contacting, or (iii) both; measuring reference quantities of a plurality of reference proteins in a reference sample by: (i) performing mass spectrometry using the plurality of reference proteins, without contacting the plurality of reference proteins with the surface, to obtain the reference quantities, such that the reference quantities do not comprise the bias or the noise; processing the set of quantities to generate a first set of features that represent changes in the quantities with respect to the set of different experimental conditions; processing the set of quantities and the reference quantities to generate a second set of features that represent a quantitative difference between the quantities and the reference quantities; processing, using the machine learning algorithm, the first set of features to generate an output value; and adjusting one or more numerical parameters of the machine learning algorithm based on a loss function based at least in part on the output value and the second set of features, such that the output value accounts for the quantitative difference between the quantities and the reference quantities, thereby training the machine learning algorithm.

In some embodiments, the method further comprises: measuring initial quantities of a plurality of target proteins in a target sample, by: i. contacting the plurality of target proteins with the surface to generate a plurality of adsorbed target proteins; and ii. performing mass spectrometry (MS) using the plurality of adsorbed target proteins to obtain the initial quantities, wherein the initial quantities comprise the bias or the noise; repeating the measuring and performing mass spectroscopy using the set of different experimental conditions to generate a set of initial quantities; processing the set of initial quantities to generate a third set of features that represent changes in the initial quantities with respect to the set of different experimental conditions; processing, using the machine learning algorithm, the third set of features to generate an output value; and using the output value to adjust the initial quantities to generate adjusted quantities, wherein the adjusted quantities comprise less of the bias or the noise.

Another aspect of the present disclosure provides a computer program product comprising a computer-readable medium having computer-executable code encoded therein, the computer-executable code adapted to be executed to implement any one of the methods disclosed herein.

Another aspect of the present disclosure provides a non-transitory computer-readable storage media encoded with a computer program including instructions executable by one or more processors to implement any one of the methods disclosed herein.

Another aspect of the present disclosure provides a computer-implemented system comprising: a digital processing device comprising: at least one processor, an operating system configured to perform executable instructions, a memory, and a computer program including instructions executable by the digital processing device to perform any one of the methods disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

This patent application contains at least one drawing executed in color. Copies of this patent or patent application with color drawing(s) will be provided by the Office upon request and payment of the necessary fee.

FIG. 1 illustrates a PROTEOGRAPH™ workflow.

FIGS. 2A-2F are TEM images of silica-coated SPIONs without installed functional ligands (FIG. 2A and FIG. 2B), polymer-coated SPIONs (FIG. 2C and FIG. 2D), and surface-functionalized SPIONs (FIG. 2E and FIG. 2F).

FIGS. 3A-3E are schematic representations of the NPs: NP-1 (FIG. 3A), NP-2 (FIG. 3B), NP-3 (FIG. 3C), NP-4 (FIG. 3D), and NP-5 (FIG. 3E).

FIGS. 4A-4E are pie charts illustrating the elemental composition of each NP measured by XPS: NP-1 (FIG. 4A), NP-2 (FIG. 4B), NP-3 (FIG. 4C), NP-4 (FIG. 4D), and NP-5 (FIG. 4E).

FIGS. 5A-5C illustrate solution-phase properties measured by DLS. Bar plots from top to bottom show the hydrodynamic size (FIG. 5A), PDI (FIG. 5B), and zeta potential (FIG. 5C) of each NP sample in the panel.

FIG. 6 illustrates NP-protein interactions and competitive binding at different protein to NP-surface ratio (P/NP) ratios (not to scale).

FIG. 7 illustrates a distribution of the plasma concentration of proteins that are identified at different NP dilutions (based on the estimated log 10 ng/ml values in HPPP reference library). Protein groups were filtered for complete identification in all replicates. Boxes show 25% (lower hinge), 50%, and 75% quantiles (upper hinge). Whiskers indicate observations equal to or outside hinge±1.5*interquartile range (IQR).

FIG. 8 illustrates the number of identified protein groups at different NP dilution ratios and in neat plasma. The error bars show standard deviation across replicates. Lines show a regression based on the y=a*log (x+0.01)+k formula.

FIG. 9 illustrates the number of protein groups measured at five-NP panel level below given coefficient of variation (CV) for different NP dilution ratios.

FIG. 10 illustrates bootstrapped fuzzy C-means clustering on the protein group intensities using 3 centers, resulting in an up trending (cluster 1), constant (cluster 2), and down trending (cluster 3) set of proteins across the dilution series. Proteins were filtered prior to this analysis.

FIG. 11 shows violin plots indicating normalized intensities (reference data base) of proteins found in clusters 1-3. Kruskall-Wallis test indicates a difference between means between cluster abundances, and a post-hoc Mann-Whitney U-test p<0.001 indicate cluster 1 and cluster 3 abundances are significantly lower and higher respectively than cluster 2. Boxes show 25% (lower hinge), 50%, and 75% quantiles (upper hinge). Whiskers indicate observations equal to or outside hinge±1.5*interquartile range (IQR). Outliers (beyond 1.5*IQR) are not plotted.

FIG. 12 illustrates a comparison of the change in protein intensities between low P/NP conditions that results in weak Vroman effect and high P/NP conditions that increase competition of particle binding area and engender a strong Vroman effect. The y-axis shows log 10 intensity ratios between 100× and 1× P/NP. The x-axis shows log 10 intensity ratios between 2× and 1× P/NP. The diagonal indicates unchanged protein intensities between the strong and weak Vroman effect. Points above the dash line indicate enrichment and points below the dash line indicate depletion of proteins. The 25 highest abundance proteins based on the HPPP database are shown as triangles.

FIGS. 13A-13C illustrate an enrichment analysis of the increasing and decreasing protein clusters composited into a volcano plot are shown: S7 volcano plot (FIG. 13A), S118 volcano plot (FIG. 13B), and S229 volcano plot (FIG. 13C). Upper half of the plot Log2_Odds>0: Over-Representation Analysis on cluster 1 for each NP highlighting Pfam, Uniport keywords, and KEGG Pathway terms with BH corrected p-value<0.05. Lower half of the plot Log2_Odds<0: over-representation analysis on cluster 3 for each NP highlighting Pfam, Uniport keywords, and KEGG Pathway terms with BH corrected p-value<0.05.

FIG. 14 is a heatmap illustrating the z-score of genes associated with FDA-approved protein biomarkers and drug targets as well as those that have functional annotations related to hormone signaling, cytokine/chemokine signaling, and inflammation identified across the panel of 5NPs in different NP dilution ratios. Protein groups were filtered for complete features.

FIG. 15 illustrates a fold coverage increase using particles compared to neat plasma for each category at each NP dilution ratio. Protein groups were filtered for complete features.

FIG. 16 is a density estimation showing predicted rank abundances (x) derived from HPPP for proteins identified across the panel of 5 NPs at P/NP 100 that are annotated as FDA approved biomarkers, protein drug targets, or both.

FIG. 17 illustrates an accumulation of the protein drug targets across the dynamic range (rank). The line shows extrapolation of protein drug targets accumulation from the first 500 proteins to the next 1,500 proteins.

FIG. 18 illustrates an accumulation of FDA biomarkers across the dynamic range (rank). The line shows extrapolation of biomarker accumulation from the first 500 proteins to the next 1,500 proteins suggesting an additional about 150 novel biomarkers among the top 2000 plasma proteins.

FIG. 19 illustrates estimated effects of NP incubation time and concentration variation on protein corona composition. Experiment design: The plasma proteome was enriched by selected NPs (NP-4, NP-5, and NP-3) using all combinations of specified incubation times and NP concentrations and measured on MS in DIA mode.

FIG. 20 illustrates the result of 2D UMAP of MS analysis. Each dot represents 1 MS run. Experiments with the same incubation time or NP concentration are connected by the solid or dashed lines, respectively. The distance between the MS runs reflects the similarity of protein intensities measured in these experiments.

FIG. 21A illustrates 3D scatter and surface plot of BMP1 protein intensities across different P/NP ratios and corona formation times.

FIG. 21B illustrates 2D UMAP of the protein-NP dynamic profiles. Each dot represents the intensities of a given protein group across all incubation times and NP concentrations. The distance between the points reflects the similarity of protein-NP dynamics.

FIGS. 22A and 22B illustrate the correlation between the neat protein intensity (x-axis) and the protein intensity measured by using the reference NP condition (P/NP=10/1, 1 h incubation time) (y-axis; FIG. 22A) or the predicted protein intensity (y-axis; FIG. 22B), respectively.

FIGS. 23A and 23B illustrate a bootstrapped analysis of Pearson correlation between log-transformed protein intensities (predicted and measured using standard NP protocol) and log-transformed plasma protein intensities (FIG. 23A, correlations are different with p-value<10⁻³³ significance) or log-transformed HPPP protein abundance estimates (FIG. 23B; p-value<10⁻³⁰). For significance testing, Mann-Whitney test was applied to the correlations calculated for 100 subsets of 100 randomly selected (without replacement) protein groups.

FIG. 24 illustrates the distribution of the plasma protein intensities predicted using the protein-NP dynamic profiles (upper half), and the distribution of the protein intensities measured using the reference condition (P/NP=10/1, 1 h incubation time) (lower half). The vertical dashed line shows the intensity with 50% probability to detect the protein.

FIGS. 25A-25J are enrichment analyses of the increasing and decreasing protein clusters. FIGS. 25A-25E: Over-Representation Analysis on cluster 1 for each NP highlighting Pfam, Uniport keywords, and KEGG Pathway terms with BH corrected p-value<0.05. FIGS. 25F-25J: over-representation analysis on cluster 3 for each NP highlighting Pfam, Uniport keywords, and KEGG Pathway terms with BH corrected p-value<0.05.

FIGS. 26A-26J illustrate a comparison of P/NP 1× to 100× (y-axis) plotted against rank in a reference database on the left, proteins with increasing intensities with P/NP ratio (FIGS. 26A-26E) and on the right proteins with decreasing intensities (FIGS. 26F-26J).

FIGS. 27A-27E illustrate a set of proteins across the dilution series. Bootstrapped fuzzy C-means clustering was performed on the protein group intensities using 3 centers, resulting in an increasing (cluster 1), constant (cluster 2), and decreasing (cluster 3). Proteins were filtered prior to that analysis.

FIGS. 28A-28E are boxplots indicating the isoelectric points of all proteins contained in cluster 1. Whiskers indicate observations equal to or outside hinge±1.5*interquartile range (IQR). Outliers (beyond 1.5*IQR) are not plotted.

FIGS. 29A-29E are violin plots indicating higher normalized intensities of proteins found in cluster 3 as compared to cluster 1. One-way Kruskal-Wallace test indicates that the protein intensities contained in all three clusters are significantly different. Wilcox-test indicates cluster 1 and cluster 3 contain proteins with significant differences in their normalized intensities as defined by the HPP dataset. Boxes show 25% (lower hinge), 50%, and 75% quantiles (upper hinge). Whiskers indicate observations equal to or outside hinge±1.5*interquartile range (IQR). Outliers (beyond 1.5*IQR) are not plotted.

FIGS. 30A-30E illustrate divergence of select protein intensities as P/NP is increased. Protein intensities at P/NP 2× are plotted against the same protein at to 100× (y-axis). Proteins bellowing to the top 40 within the dataset as a reference database are indicated as triangles. The red line indicates the manifold where no differences can between detected between proteins are different P/NP ratios. Points above the line indicate enrichment and points below the line indicate decreasing intensities with P/NP ratio.

FIGS. 31A-31B illustrate two approaches for stratifying particle-based assay data.

FIGS. 32A-32F illustrate high viability of Vroman effect regimes across proteome: BMP1 (FIG. 32A), C4A (FIG. 32B), ORM1 (FIG. 32C), FBN2 (FIG. 32D), KLKB1 (FIG. 32E), and CXCL12 (FIG. 32F).

FIG. 33B illustrates a predicted log₂ intensity as a function of P/NP ratio based on the NP-protein data in FIG. 33A. One can train on one protein and then can apply to another related protein.

FIG. 34A illustrates a regression loss to be at least partially optimized when a reference measurement is available. FIG. 34B illustrates a logistic loss to be at least partially optimized when a reference measurement is not available.

FIG. 35A is a schematic illustration of competitive bindings of protein A and protein B to a NP and shows their ordinary differential equation models. FIG. 35B illustrates a C4A modeling based on the ordinary differential equation models.

FIG. 36A shows a surface, in accordance with some embodiments. A surface may be functionalized at one or more regions for capturing biomolecules. FIG. 36B shows a surface in accordance with some embodiments. A surface may comprise one or more wells or depressions for capturing biomolecules. For example, a functionalized surface may be disposed in a 96 well plate or a 384 well plate. FIG. 36C shows a surface in accordance with some embodiments. A surface may be disposed on one or more particles. In some embodiments, the one or more particles may be disposed in one or more wells or depressions. FIG. 36D shows a surface in accordance with some embodiments. A surface may be disposed on a plurality of particles packed in a channel or a porous material disposed in a channel. FIG. 36E shows a surface, in accordance with some embodiments. A surface may be disposed on an inner surface of a channel. FIGS. 36F-361 show surfaces in accordance with some embodiments. A surface may comprise 1, 2, 3, 4 or any number of distinct surface regions. In some embodiments, a surface may be disposed on a particle. In some embodiments, a particle may be a porous particle.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

Introducing a nanoparticle (NP) or other surfaces into a biofluid, such as blood plasma, can lead to the formation of a selective and reproducible protein corona at the nano-bio interface driven by a combination of protein-surface affinity, protein abundance, and protein-protein interactions. These interactions can be exploited to interrogate the entire plasma proteome at scale and depth without the inherent bias of targeted analyte-specific probes (e.g., antibodies or aptamers). When introduced into a biological matrix, proteins may assemble on surfaces to form a protein corona via physical adsorption and/or electrostatic interactions. Without requiring a presence of a specific entity that is configured for binding to a singular specific protein (e.g., as in immunoassays), the nanoparticles can allow dynamic range compression of proteins bound to the nanoparticle surfaces while capturing a wide variety of proteins. In other words, the relative abundance of proteins in the sample can be modified on the nanoparticle surfaces, such that the rare proteins are relatively more abundant, and the highly abundant proteins are relatively less abundant compared to the original sample.

At preequilibrium, the protein corona composition can be driven by the relative proximity of proteins that diffuse to interacting moieties on the particle surface. As such, proteins with high abundance can dominate the initial corona composition. At equilibrium, governed by thermodynamics, high-abundance low-affinity proteins on the NP surface can be displaced by low-abundance high-affinity proteins (Vroman effect), which may lead to compression of the dynamic range. The competition between proteins for binding to a surface (e.g., the Vroman effect) can play an important role in protein corona composition, and surfaces can be tuned with different functionalizations to enhance and differentiate protein selectivity. The quantitative composition of protein coronas thus can depend on the physicochemical properties of the surfaces, the presence and abundance of proteins with compatible surface epitopes, and the competition of proteins for binding.

The compression of the dynamic range can confer significant advantages in determining the biomolecule composition in biofluids such as human plasma. Human plasma contains protein species over a dynamic range that exceeds 12 orders of magnitude, where the top few proteins (e.g., albumin, transferrin, complement proteins, apolipoproteins, and alpha-2-macroglobulin) comprise 95% of the mass of protein in the plasma, and most of the protein species comprise the remaining 5%. Some of the protein species exist in the nanograms per milliliter ranges (e.g., transforming growth factor beta-1-induced transcript 1 protein at ˜10 ng/ml; fructose-bisphosphate aldolase A at ˜20 ng/ml; thioredoxin at ˜18 ng/ml; and L-selectin at ˜92 ng/ml), and some proteins are expected to present at level even beneath that range. Liquid chromatography coupled with mass spectrometry (LC-MS) or tandem mass spectrometry (LC-MS/MS) can be used to identify protein species in plasma; however, due to the stochastic nature of the methods, only a fraction of ionic species that are generated at a time from a given sample may be selected for acquiring mass spectra. As a result, the species that are highly abundant compared to the rare species can generate a signal that overwhelms signal from rare species. Compressing the dynamic range of protein species in a sample can allow rare proteins to comprise a higher fraction of ionic species, thereby allowing higher probability for detecting those rare proteins in a MS experiment. This process, incorporated within the Proteograph™ proteomics platform, may offer superior plasma profiling performance in terms of depth and breadth, compared to conventional shallow and deep workflows.

In some cases, a dynamic range may be compressed by at least about 0.1, 0.5, 1, 1.5, 2, 3, 4, 5, 6, 7, 8, 9, 10, 11, 12, 13, 14, or 15 orders of magnitude. In some cases, a dynamic range may be compressed at most about 1, 2, 3, 4, 5, 6, 7, 8, 9, 10, 11, or 12 orders of magnitude. In some embodiments, a dynamic range may be compressed by about 2 to 12 orders of magnitude. In some embodiments, a dynamic range may be compressed by about 6 to 10 orders of magnitude.

In some cases, it may be desirable to obtain quantities of proteins in a sample before the dynamic range compression using nanoparticles. In some aspects, the present disclosure provides a process which can comprise measuring quantities of proteins using nanoparticles to compress the dynamic range, and then using a machine learning algorithm to decompress the measured quantities to the quantities that are expected in the sample before dynamic range compression.

In some aspects, the present disclosure provides a computer-implemented method for training a machine learning algorithm for molecule quantification. An input dataset for training the machine learning algorithm can comprise one or more features that represent changes in quantities for a plurality of molecules with respect to one or more physicochemical parameters. The changes can be measured in at least a first condition (e.g., an experimental condition). The input dataset can be processed by the machine learning algorithm to generate an output value. One or more numerical parameters of the machine learning algorithm can be adjusted by minimizing a loss function based at least in part on the output value, such that the output value accounts for a difference between (i) the quantities for at least a portion of the molecules and (ii) reference quantities for a plurality of reference molecules, wherein the reference quantities are measured in at least a second condition.

The trained machine learning algorithm can be used to quantify molecules using different datasets. In some aspects, the present disclosure provides a computer-implemented method for quantifying a molecule using a machine learning algorithm. An input dataset comprising one or more features can be provided. The one or more features can represent a quantity of the molecule measured in at least a first condition. The input dataset can be processed by the machine learning algorithm to generate an adjusted quantity. The adjusted quantity can be a predicted quantity of the molecule as if the molecule was measured in a second condition.

### Methods for Molecule Quantification

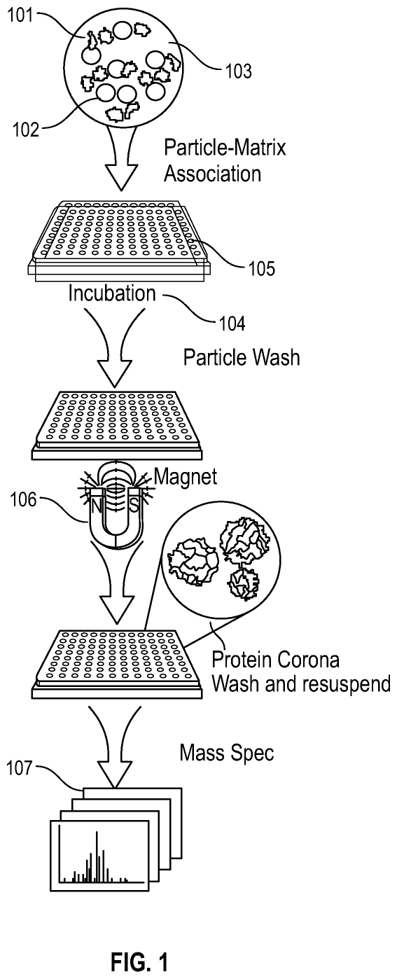

In some embodiments, the machine learning algorithm can be trained for protein quantification. An input dataset for training the machine learning algorithm can be generated by measuring quantities of proteins in a sample using a surface for binding proteins (e.g., nanoparticles). The quantities can be measured by, for example, following the process illustrated in FIG. 1. Biomolecules, such as proteins (101), in a sample can be contacted (103) with a surface, such as nanoparticles (102), such that the proteins are adsorbed on the surface. The proteins can be incubated (104) with the surface in partitions or wells (e.g., 96 well-plate; 105). The surfaces can be particle surfaces, wherein the particles can comprise a magnetic material that can be manipulated with a magnet (106) to facilitate separating, washing, and other relevant processing steps disclosed elsewhere herein. Mass spectrometry (MS; 107) or other assays can be performed using the adsorbed proteins to obtain quantities of the proteins in the sample. The quantities can comprise a deviation or a noise that are introduced by contacting the proteins with the surface.

The input dataset can be generated by repeating the measurement of the proteins varying experimental conditions. The repeated measurements can generate a set of quantities for the input dataset, wherein the set of varying experimental conditions can be varied, for example, in (i) ratios of the surface area to the plurality of proteins, (ii) incubation time used for the contacting, or (iii) both. Other experimental conditions that can influence the thermodynamics and/or kinetics of the adsorption between of the proteins and the surface can be varied as well.

A reference dataset can be generated by measuring quantities of proteins in a reference condition. For example, a reference condition may be performing mass spectrometry using the sample directly (e.g., without contacting it with the surface described above). In another example, the reference condition may be performing mass spectrometry after contacting the sample with a reference surface. In yet another example, the reference condition may be measuring quantities of the proteins using another readout method or technology, e.g., ELISA, protein sequencing, antibody-based quantification, etc. The reference condition can be any condition, where an experimenter can aim to adjust quantities obtained from one condition to the quantities that could or would have been obtained at the reference condition. For example, the quantity of proteins within a biomolecule corona formed by incubating plasma with one or more particles may be adjusted to quantities of the proteins measured directly from plasma (e.g., by performing MS or ELISA on neat plasma). The quantities obtained in the reference condition may be subject to fewer deviations or less noise, or different kinds of deviation or noise. When the reference condition is performing mass spectrometry without contacting proteins with a surface, then the quantities obtained would not comprise the deviation or the noise that would be introduced by the contact between the proteins and the surface.

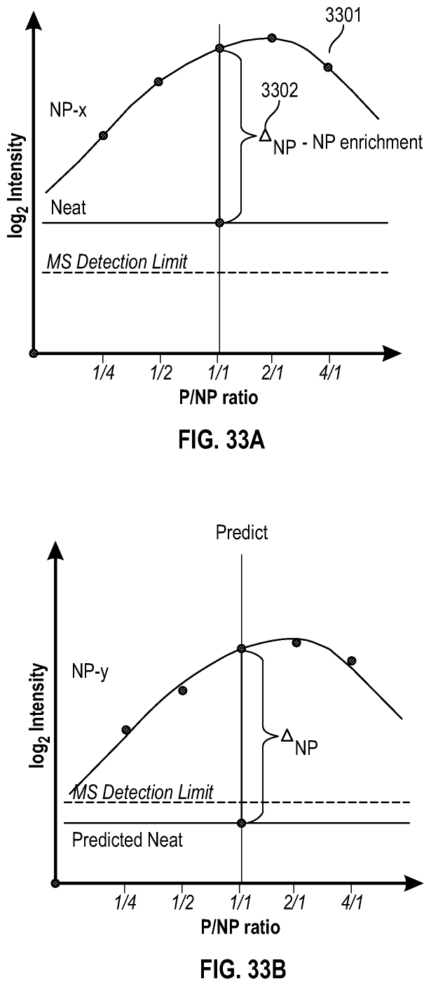

The input dataset can be processed to generate features that represent changes in the quantities with respect to the set of varying experimental conditions. For example, FIG. 33A shows a curved plot (3301) that can provide information that indicates a derivative of a quantity for a protein with respect to a change in an experimental parameter that influences binding of a protein to a surface (e.g., by changing the thermodynamics and/or kinetics of protein binding). The input dataset and the reference dataset can be processed to generate features that represent a quantitative difference between the quantities and the reference quantities. For example, FIG. 33A shows a feature that represents the quantitative difference (3302) between the quantity of protein measured using a surface versus a reference condition, the reference condition being measuring protein quantity from a sample without using the surface.

The input quantities may comprise quantities of the molecules (e.g., proteins) directly or other quantities related to or derived from molecule quantities. For example, in cases where protein quantities are determined by performing native or top down MS, the input quantities may comprise protein quantities. Alternatively, or additionally, the input quantities may comprise peptide quantities, such as in cases where the quantified proteins are digested prior to MS analysis (e.g., bottom up proteomics or middle down proteomics). For example, peptide quantities can be used to infer an input protein quantity by averaging, selecting the median, or selecting the maximum measured quantity from the peptides derived from the protein. The skilled person, guided by the teachings in the present application, will appreciate that other methods of inferring the protein quantity from peptide quantity can be used.

A machine learning algorithm can be trained using the features such that the machine learning algorithm generates output values that account for quantitative differences between the quantities and the reference quantities. In some embodiments, the quantitative differences can comprise deviation and/or noise associated with protein quantities measured using a surface. Without being bound to a particular theory, it is contemplated that the derivative of the quantity of a protein with respect to a change in one or more experimental parameters, (e.g., ∂Q/∂X, where ∂ denotes a partial derivative, Q denotes a quantity, and X denotes some parameter), can provide important information related to the thermodynamic, kinetic or other behavior of the protein (and in appropriate cases, of the environment) that influences binding of the protein to a surface. For instance, varying incubation time can provide time-dependent information (e.g., ∂Q/∂t, where t denotes time). Varying protein to nanoparticle ratio can provide thermodynamic information (e.g., ∂Q/∂μ, where μ denotes chemical potential). By providing this information to a machine learning algorithm as numerical features that the machine learning algorithm can process, the machine learning algorithm can learn patterns from the derivative information (e.g., information representing relative or absolute changes) that are associated with certain types or levels of deviations and/or noise associated with the quantity measured using the surface. Once trained, the machine learning can be used to adjust the quantity measured using the surface. The adjusted quantity may be closer to the actual quantity of a protein in the sample than originally measured using a surface.

In some aspects, the present disclosure provides a computer-implemented method for using the machine learning algorithm for protein quantification. A trained machine learning algorithm can be used to process features from new measurements of protein quantities (e.g., measurements extraneous to the input dataset used for training), to adjust quantities of the new measurements. For instance, the computer-implemented method can comprise measuring or receiving initial quantities for target proteins in a target sample. The initial quantities may be obtained, in some embodiments, by contacting target proteins with a surface to generate adsorbed target proteins. The adsorbed target proteins can be quantified using mass spectrometry, which can generate initial quantities of the target proteins. The measurement can be repeated using varying experimental conditions to obtain derivative information for the target proteins. The derivative information can be processed to generate numerical features to be used as input for the machine learning algorithm. The numerical features can be processed using the machine learning algorithm to produce output values. The output values can be used to adjust the initial quantities. The adjusted quantities can be less affected by deviations and/or noise introduced by contacting the target proteins with the surface. As result, the adjusted quantities can be closer to the actual quantities of the target proteins in the target sample. In some cases, a second scale of the method or device comprises a deviation. In some cases, the deviation can be based on a number of charges and flyability of a molecule. In some cases, a first scale of the predicted quantity is different from a second scale of the method or device used to generate the machine learning model.

A target protein (or molecule) can be among the proteins used to generate the training dataset. Alternatively, a target protein can be excluded from the proteins (or molecules) used to generate the training dataset. Without being bound to a particular theory, it is expected that when the derivative information of two different proteins are similar or identical, the adjustment to the protein quantities can be similar or identical. For instance, even if two proteins may have different chemical structures, if their thermodynamic and/or kinetic behavior as elucidated by the derivative information is similar or identical, then the adjustment for the two proteins can also be similar or the same. Therefore, even when the machine learning algorithm may be “blind” to the chemical structure of a protein, the machine learning algorithm can be used to adjust a quantity of the protein when the machine learning algorithm has been trained on sufficient examples of derivative information. Thus, the quantity of the target protein can be adjusted by providing its derivative information to the machine learning algorithm even when the target protein is not in the training dataset used to train the machine learning algorithm.

While the above example has been described using proteins as an example, those skilled in the art will recognize that other classes of molecules can similarly quantified. Some embodiments of the surfaces contemplated above binds to the proteins via adsorption. Adsorption is a phenomenon that can occur with various molecules, including lipids, nucleic acids, sugars, small molecules, polymers, salts.

While the above example has been described using experimental parameters such as incubation time with a surface and available surface area of the surface as examples, those skilled in the art will recognize that other parameters can be varied to provide salient information for similar purposes. The derivative information (e.g., ∂Q/∂X) could be interrogated by varying any physicochemical parameters that can influence the binding with a surface. For instance, changes in the solvent environment (e.g., temperature, dielectric constant of the solvent, ionic strength, pH, crowding agent concentration, concentrations of other molecules, etc.) and characteristics of the surface (e.g., surface area, pKa, roughness, curvature, hydrophobicity, etc.) may provide the derivative information. In some embodiments, the derivative information may be based on varying two or more physicochemical properties (e.g., two, three, four, five, six, or more).

While the above example has been described using adsorption to a surface followed by MS as an example, those skilled in the art will recognize that other technologies for measuring molecule quantities can be used. For instance, quantities of proteins detected by immunoassays (which can rely on specific binding of proteins) can also have relevant derivative information. Protein sequencing technologies can also have relevant derivative information.

In some embodiments, the systems and methods disclosed herein can be used to adjust quantities of proteoforms detected in a sample. The proteoforms can include, but are not limited to, splicing variant, post-translation cleavage, amino acid modification, such as acylation (e.g., acetylation), phosphorylation, ubiquitinylation, glycosylation, oxidation, and the like. For example, quantification of peptides derived from the same protein may be used to infer the quantity of different splicing variants. The machine learning algorithm may receive input datasets with peptide quantities and reference datasets with quantities for the proteoforms.

In some aspects, the present disclosure provides a computer-implemented method for training a machine learning algorithm for molecule quantification. The computer-implemented method can comprise providing an input dataset comprising one or more features. The one or more features can represent changes in quantities for a plurality of molecules with respect to one or more physicochemical parameters. The changes can be measured using at least a first condition.

The computer-implemented method can comprise processing, using the machine learning algorithm, the input dataset to generate an output value. The computer-implemented method can comprise adjusting one or more numerical parameters of the machine learning algorithm. The adjusting can be based on a loss function based at least in part on the output value. The adjusting can result in the output value accounting for a difference between (i) the quantities for at least a portion of the molecules and (ii) reference quantities for a plurality of reference molecules, wherein the reference quantities are measured using at least a second condition.

### Measurement

In some embodiments, a measurement can be preceded by binding a plurality of molecules to a surface. The surface can comprise a sensor element surface. The sensor element surface can comprise a particle surface. The particle surface can be a nanoparticle surface. The particle surface can be a microparticle surface. The particle surface can comprise pores. The binding can comprise adsorption. The binding can be non-specific. The binding can be specific. The plurality of molecules can form a corona on the particle surface.

In some embodiments, measured quantities comprise measured intensities. In some embodiments, reference quantities comprise measured intensities. The measured intensities can be obtained using a variety of methods and/or instrumentation. The measured intensities can comprise mass spectrometry (MS) intensities. The MS intensities can comprise peptide intensities, protein group intensities, or both. The MS intensities can comprise small molecule intensities. The MS intensities can be based on data-independent acquisition (DIA) MS, data-dependent acquisition (DDA) MS, or both. The MS intensities can be based on liquid-chromatography tandem mass spectrometry (LC-MS/MS). The measured intensities can be obtained using a nanopore sensor. The measured intensities can be obtained using an immunoassay. The measured intensities can be obtained using a nucleic acid sequencer. The measured intensities can comprise fluorescence signals. The measured intensities can comprise an induced current. In some embodiments, the measured intensities can be obtained using gas phase separation.

The measured intensities can be obtained using an antibody. The measured intensities can be obtained by binding a molecule in the plurality of molecules to an antibody. The measured intensities can be obtained by binding the molecule to a pair of antibodies. The pair of antibodies can comprise complementary single-stranded nucleic acid sequences attached thereto. When the pair of antibodies bind to the molecule, the complementary nucleic acids can hybridize to form a double stranded nucleic acid. The double stranded nucleic acid can be configured to form a binding complex with a polymerase and a plurality of nucleotides, nucleosides, nucleotide analogs, and/or nucleoside analogs to perform an amplification reaction to produce a detectable signal.

The measured intensities can be obtained using an aptamer. The aptamer can be coupled to a surface via a cleavable linker. The surface can be a particle surface. The cleavable linker can be photocleavable. The measured intensities can be obtained by contacting the molecule and the aptamer with a macromolecular competitor configured to, in a fluid composition, reduce dissociation of a complex comprising the one or more aptamers and the molecule. The macromolecular competitor can be a polyanionic macromolecule.

The measured intensities can be obtained using protein sequencing. The protein sequencing can comprise digesting the plurality of proteins to generate a plurality of protein fragments. The protein sequencing can comprise immobilizing the plurality of protein fragments to a semiconductor substrate. The protein sequencing can comprise contacting the plurality of protein fragments with a plurality of labeled recognizers. The plurality of labeled recognizers can be configured to attach to a predetermined chemical moiety in the plurality of protein fragments at the N-terminus of the plurality of protein fragments. The protein sequencing can comprise exciting the plurality of labeled recognizers to detect the plurality of labeled recognizers, thereby detecting the predetermined chemical moiety. The protein sequencing can comprise removing an amino acid from the N-terminus of the plurality of protein fragments. The protein sequencing can comprise contacting the plurality of protein fragments with a second plurality of labeled recognizers. The protein sequencing can comprise exciting the second plurality of labeled recognizers to detect a second amino acid from the N-terminus of the plurality of protein fragments, thereby performing the protein sequencing.

In some embodiments, the measured intensities can be obtained using a neat measurement condition. In some embodiments, the neat measurement condition does not comprise binding the molecule to the surface. In some embodiments, the measured intensities can be obtained using liquid chromatography mass spectrometry (LC-MS) with a gradient length equal to or greater than 10, 20, 30, 40, 50, 60, 70, 80, 90, 100, 110, or 120 minutes. In some embodiments, the measured intensities can be obtained using LC-MS with a gradient length less than or equal to 10, 20, 30, 40, 50, 60, 70, 80, 90, 100, 110, or 120 minutes.

In some embodiments, a machine learning algorithm is trained using an input dataset. The input dataset can comprise the quantities, the reference quantities, a plurality of differences between the quantities and the reference quantities, or any combination thereof. In some embodiments, the reference molecules are the same as the at least the portion of the molecules used to generate the input dataset. In some embodiments, the reference quantities of the reference molecules are derived from the same sample as the at least the portion of the molecules used to generate the input dataset.

The reference quantities can be set in various ways. In some embodiments, the reference quantities comprise average abundance values of the molecules over a plurality of samples. In some embodiments, the average abundance values are concentration values, intensities values, or relative abundance values. In some embodiments, the reference quantities comprise an aggregate of measurements of samples. In some embodiments, the reference quantity of a reference molecule in the reference molecules is based on a reference signal of another molecule.

In some cases, the reference quantities can be obtained from databases. For example, the reference quantities can be obtained from the Human Plasma Proteome Project (HPPP) or the Proteomics Identifications Database (PRIDE).

In some cases, the reference quantities can be obtained from labeled molecules in a sample. For example, proteins adsorbed on the surface can be labeled with tandem-mass-tag (TMT; e.g., isobaric or non-isobaric labeling such as iTRAQ) and be mixed with TMT labeled proteins obtained from a neat extraction (e.g., proteins without contacting with a surface). In some cases, a sample of known composition can be labeled (e.g., via Stable Isotope Labeling by Amino Acids in Cell Culture, “SILAC”) and be mixed with proteins adsorbed on the surface. Signals obtained from the reference quantities (e.g., quantities of proteins from a sample of known composition, or quantities of proteins measured from a neat extraction method) can be used.

### Physicochemical Parameters

A machine learning algorithm can be trained using an input dataset comprising one or more features that represent changes in quantities for a plurality of molecules with respect to one or more physicochemical parameters (which can provide derivative information). The changes in the quantities for the plurality of molecules with respect to one or more physicochemical parameters can be obtained by measuring the quantities while varying the one or more physicochemical parameters. The one or more physicochemical parameters can comprise: sample to surface ratio, incubation time, pH, salt concentration, ionic strength, solvent composition, solvent dielectric constant, crowding agent concentration, temperature, sample composition, surfactant concentration, concentration of enzymes, activity of enzymes, chemical reactions, concentrations of small molecules, surface chemistry (e.g., hydrophobicity, charge, polymeric, chemical moieties, etc.) or any combination thereof.

The sample to surface ratio can comprise (i) volume of sample to surface area of the surface, (ii) volume of sample to mass of a substrate comprising the surface, (iii) mass of sample to surface area of the surface, or (iv) mass of sample to mass of the substrate comprising the surface.

The one or more physicochemical parameters can comprise a ratio of surface area of the surface to a volume of a sample comprising the plurality of molecules. The ratio can be at least 0.0001, 0.0005, 0.001, 0.005, 0.01, 0.05, 0.1, 0.5, 1, 5, or 10 cm² per μL. The ratio can be at most 0.0001, 0.0005, 0.001, 0.005, 0.01, 0.05, 0.1, 0.5, 1, 5, or 10 cm² per μL. The one or more physicochemical parameters can comprise a ratio of surface area of the surface to a concentration of the plurality of molecules in a sample. The ratio can be at least 0.0001, 0.0005, 0.001, 0.005, 0.01, 0.05, 0.1, 0.5, 1, 5, or 10 cm² per μg/μL. The ratio can be at most 0.0001, 0.0005, 0.001, 0.005, 0.01, 0.05, 0.1, 0.5, 1, 5, or 10 cm² per μg/μL. The one or more physicochemical parameters can comprise a ratio of surface area of the surface to a mass of the plurality of molecules in a sample. The ratio can be at least 0.0001, 0.0005, 0.001, 0.005, 0.01, 0.05, 0.1, 0.5, 1, 5, or 10 cm² per μg. The ratio can be at most 0.0001, 0.0005, 0.001, 0.005, 0.01, 0.05, 0.1, 0.5, 1, 5, or 10 cm² per μg. The one or more physicochemical parameters can comprise a ratio of mass of a substrate comprising the surface to a volume of a sample comprising the plurality of molecules. The ratio can be at least 0.0001, 0.0005, 0.001, 0.005, 0.01, 0.05, 0.1, 0.5, 1, 5, or 10 μg/μL. The ratio can be at most 0.0001, 0.0005, 0.001, 0.005, 0.01, 0.05, 0.1, 0.5, 1, 5, or 10 μg/μL. The one or more physicochemical parameters can comprise a ratio of mass of a substrate comprising the surface to a concentration of the plurality of molecules in a sample. The ratio can be at least 0.0001, 0.0005, 0.001, 0.005, 0.01, 0.05, 0.1, 0.5, 1, 5, or 10 μL⁻¹. The ratio can be at most 0.0001, 0.0005, 0.001, 0.005, 0.01, 0.05, 0.1, 0.5, 1, 5, or 10 μL⁻¹. The one or more physicochemical parameters can comprise a ratio of mass of a substrate comprising the surface to a mass of the plurality of molecules in a sample. The ratio can be at least 0.0001, 0.0005, 0.001, 0.005, 0.01, 0.05, 0.1, 0.5, 1, 5, or 10. The ratio can be at most 0.0001, 0.0005, 0.001, 0.005, 0.01, 0.05, 0.1, 0.5, 1, 5, or 10. The ratio can be varied in a number of experiments to obtain derivative information of the plurality of molecules.

The one or more physicochemical parameters can comprise an incubation time for the plurality of molecules to the surface. The incubation time can be at least 1, 15, 30, 45, or 60 seconds. The incubation time can be at least 1, 15, 30, or 60 minutes. The incubation time can be at least 1, 2, 3, 4, 8, 12, 16, 20, or 24 hours. The incubation time can be at least 1, 2, 3, 4, 5, 6 or 7 days. The incubation time can be at most 1, 2, 3, 4, 5, 6 or 7 days. The incubation time can be at most 1, 2, 3, 4, 8, 12, 16, 20, or 24 hours. The incubation time can be at most 1, 15, 30, or 60 minutes. The incubation time can be at most 1, 15, 30, or 60 seconds. The incubation time can be varied in a number of experiments to obtain derivative information of the plurality of molecules.

The pH can be at least 1, 2, 3, 4, 5, 6, 7, 8, 9, 10, 11, 12, 13, or 14. The pH can be at most 1, 2, 3, 4, 5, 6, 7, 8, 9, 10, 11, 12, 13, or 14. The ion concentration can be at least 0.1, 0.2, 0.3, 0.4, 0.5, 0.6, 0.7, 0.8, 0.9, 1, 1.5, 2, 2.5, 3, 3.5, 4, 4.5, or 5 mols per liter. The ion concentration can be at most 0.1, 0.2, 0.3, 0.4, 0.5, 0.6, 0.7, 0.8, 0.9, 1, 1.5, 2, 2.5, 3, 3.5, 4, 4.5, or 5 mols per liter. In some cases, a solvent can comprise a salt comprising LiF, LiCl, LiBr, LiI, Li₂SO₄, BeF₂, BeCl₂, BeBr₂, BeI₂, BeSO₄, NaF, NaCl, NaBr, NaI, Na₂SO₄, MgF₂, MgCl₂, MgBr₂, MgI₂, MgSO₄, KF, KCl, KBr, KI, K₂SO₄, CaF₂, CaCl₂, CaBr₂, CaI₂, KSO₄, NH₄F, NH₄Cl, NH₄Br, NH₄I, (NH₄)₂SO₄, or any combination thereof. The solvent can comprise water, alcohol, ketone, a buffer, or any combination thereof. In some cases, a solvent may comprise various acids or bases. In some cases, an acid may comprise hydrochloric, acetic acid, sulfuric acid, nitric acid, citric acid, or any combination thereof. In some cases, a base may comprise NaOH, KOH, Ca(OH)₂, NH₄OH, or any combination thereof. The solvent dielectric constant can be at least 1, 5, 10, 15, 20, 25, 30, 35, 40, 45, 50, 55, 60, 65, 70, 75, or 80. The solvent dielectric constant can be at most 1, 5, 10, 15, 20, 25, 30, 35, 40, 45, 50, 55, 60, 65, 70, 75, or 80. The temperature can be at least −20, −15, −10, −5, 0, 5, 10, 15, 20, 25, 30, 35, 40, 45, 50, 55, 65, 70, 75, 80, 85, 90, 95, or 100° C. The temperature can be at most −20, −15, −10, −5, 0, 5, 10, 15, 20, 25, 30, 35, 40, 45, 50, 55, 65, 70, 75, 80, 85, 90, 95, or 100° C. The solvent environment for binding can be varied in a number of experiments to obtain derivative information of the plurality of molecules.

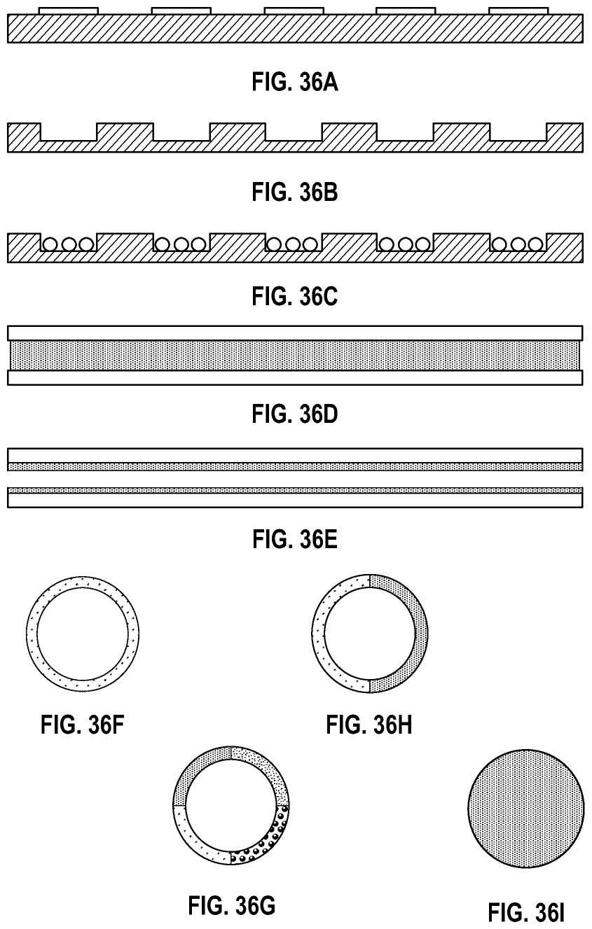

The one or more physicochemical parameters can comprise different types of surfaces. FIG. 36 shows types of surfaces, in accordance with some embodiments. A surface may be functionalized at one or more regions for capturing biomolecules. A surface may comprise one or more wells or depressions for capturing biomolecules. For example, a functionalized surface may be disposed in a 96 well plate or a 384 well plate. A surface may be disposed on one or more particles. In some embodiments, the one or more particles may be disposed in one or more wells or depressions. A surface may be disposed on a plurality of particles packed in a channel or a porous material disposed in a channel. A surface may be disposed on an inner surface of a channel. A surface may comprise 1, 2, 3, 4 or any number of distinct surface regions. In some embodiments, a surface may be disposed on a particle. In some embodiments, a particle may be a porous particle.

A surface may comprise a wide array of physical properties. A physical property of a surface may include surface charge, hydrophobicity, hydrophilicity, acidity, basicity, surface topography, surface curvature, porosity, shape, and any combination thereof.

A surface functionalization may comprise a polymerizable functional group, a positively or negatively charged functional group, a zwitterionic functional group, an acidic or basic functional group, a polar functional group, or any combination thereof. A surface functionalization may comprise carboxyl groups, hydroxyl groups, thiol groups, cyano groups, nitro groups, ammonium groups, alkyl groups, imidazolium groups, sulfonium groups, pyridinium groups, pyrrolidinium groups, phosphonium groups, aminopropyl groups, amine groups, boronic acid groups, N-succinimidyl ester groups, PEG groups, streptavidin, methyl ether groups, triethoxylpropylaminosilane groups, PCP groups, citrate groups, lipoic acid groups, BPEI groups, or any combination thereof. A surface can be the surface of: micelles, liposomes, iron oxide particles, silver particles, gold particles, palladium particles, quantum dots, platinum particles, titanium particles, silica particles, metal or inorganic oxide particles, synthetic polymer particles, copolymer particles, terpolymer particles, polymeric particles with metal cores, polymeric particles with metal oxide cores, polystyrene sulfonate particles, polyethylene oxide particles, polyoxyethylene glycol particles, polyethylene imine particles, polylactic acid particles, polycaprolactone particles, polyglycolic acid particles, poly(lactide-co-glycolide polymer particles, cellulose ether polymer particles, polyvinylpyrrolidone particles, polyvinyl acetate particles, polyvinylpyrrolidone-vinyl acetate copolymer particles, polyvinyl alcohol particles, acrylate particles, polyacrylic acid particles, crotonic acid copolymer particles, polyethlene phosphonate particles, polyalkylene particles, carboxy vinyl polymer particles, sodium alginate particles, carrageenan particles, xanthan gum particles, gum acacia particles, Arabic gum particles, guar gum particles, pullulan particles, agar particles, chitin particles, chitosan particles, pectin particles, karaya tum particles, locust bean gum particles, maltodextrin particles, amylose particles, corn starch particles, potato starch particles, rice starch particles, tapioca starch particles, pea starch particles, sweet potato starch particles, barley starch particles, wheat starch particles, hydroxypropylated high amylose starch particles, dextrin particles, levan particles, elsinan particles, gluten particles, collagen particles, whey protein isolate particles, casein particles, milk protein particles, soy protein particles, keratin particles, polyethylene particles, polycarbonate particles, polyanhydride particles, polyhydroxyacid particles, polypropylfumerate particles, polycaprolactone particles, polyamine particles, polyacetal particles, polyether particles, polyester particles, poly(orthoester) particles, polycyanoacrylate particles, polyurethane particles, polyphosphazene particles, polyacrylate particles, polymethacrylate particles, polycyanoacrylate particles, polyurea particles, polyamine particles, polystyrene particles, poly(lysine) particles, chitosan particles, dextran particles, poly(acrylamide) particles, derivatized poly(acrylamide) particles, gelatin particles, starch particles, chitosan particles, dextran particles, gelatin particles, starch particles, poly-β-amino-ester particles, poly(amido amine) particles, poly lactic-co-glycolic acid particles, polyanhydride particles, bioreducible polymer particles, and 2-(3-aminopropylamino) ethanol particles, and any combination thereof.

Surfaces can comprise various functionalizations. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group.

A small molecule functionalization may comprise a reactive functional group. Non-limiting examples of the reactive functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a surface can be functionalized to carry a net positive surface charge, a net negative surface charge, an approximately neutral charge. The surface can be a zwitterionic surface.

A surface functionalization may comprise a macromolecular functionalization. A macromolecular functionalization may comprise a biomacromolecule, such as a protein or a polynucleotide (e.g., a 100-mer DNA molecule). A macromolecular functionalization may be comprise a protein, polynucleotide, or polysaccharide, or may be comparable in size to any of the aforementioned classes of species. For example, a macromolecular functionalization may comprise a volume of at least 6, 8, 10, 20, 30, 40, 50, 60, 70, 80, 90, 100, 200, 300, 400, 500, 600, 700, 800, 900, 1000, or 2000 nm³. A macromolecular functionalization may comprise a volume of at most 6, 8, 10, 20, 30, 40, 50, 60, 70, 80, 90, 100, 200, 300, 400, 500, 600, 700, 800, 900, 1000, or 2000 nm³. A macromolecular functionalization may comprise a surface area of at least 15, 30, 50, 80, 100, 200, 300, 400, 500, 600, 700, 800, 900, 1000, or 1500 nm². A macromolecular functionalization may comprise a surface area of at most 15, 30, 50, 80, 100, 200, 300, 400, 500, 600, 700, 800, 900, 1000, or 1500 nm². A macromolecular functionalization may comprise a bait molecule.

Measurements using varying physicochemical conditions can be obtained in serial, in parallel, or a combination thereof. For example, a plurality of partitions or wells can be provided, wherein one of the partitions or the wells can be configured to provide a different physicochemical condition for performing the measurement compared to another. One partition or well can comprise a different solvent, temperature, sample to surface ratio, be used with a different incubation time, etc., compared to another partition or well. In some embodiments, a control sample (e.g., a plasma standard sample) can be provided in a partition or a well to obtain reference quantities.

### Machine Learning

In some cases, quantities of molecules can be processed using methods, including, but not limited to, for example, a wide variety of supervised and unsupervised data analysis, machine learning, deep learning, modeling and clustering approaches including hierarchical cluster analysis (HCA), principal component analysis (PCA), Partial least squares Discriminant Analysis (PLS-DA), random forest, logistic regression, decision trees, support vector machine (SVM), k-nearest neighbors, naive Bayes, linear regression, polynomial regression, SVM for regression, K-means clustering, and hidden Markov models, differential equation and stochastic differential equation models, among others. In some embodiments, the machine learning algorithm may be trained to learn a latent representation of quantities of molecules. In some embodiments, the machine learning algorithm may be supervised learning algorithm.

Input features to a machine learning algorithm may comprise various kinds of information. In some cases, an input feature may comprise a value that represents a physicochemical property of a surface used to assay a biomolecule. A physicochemical property of a particle may comprise various properties disclosed herein, which includes: charge, hydrophobicity, hydrophilicity, amphipathicity, coordinating, reaction class, surface free energy, various functional groups/modifications (e.g., sugar, polymer, amine, amide, epoxy, crosslinker, hydroxyl, aromatic, or phosphate groups). In some cases, an input feature may comprise a value that represents a parameter of a given measurement. A parameter may comprise incubation conditions including temperature, incubation time, pH, buffer type, and any variables in performing a measurement disclosed herein. In some embodiments, the input datasets may include a series of quantity measurements at different conditions, but without any data representing the relative differences between the conditions.

In some cases, a clustering algorithm can refer to a method of grouping samples in a dataset by some measure of similarity. In some cases, samples can be grouped in a set space, for example, element ‘a’ is in set ‘A’. In some cases, samples can be grouped in a continuous space, for example, element ‘a’ is a point in Euclidean space with distance ‘l’ away from the centroid of elements comprising cluster ‘A’. In some cases, samples can be grouped in a graph space, for example, element ‘a’ is highly connected to elements comprising cluster ‘A’. In some cases, clustering can refer to the principle of organizing a plurality of elements into groups in some mathematical space based on some measure of similarity.

In some cases, clustering can comprise grouping any number of molecules or quantities in a dataset by any quantitative measure of similarity. In some cases, clustering can comprise K-means clustering. In some cases, clustering can comprise hierarchical clustering. In some cases, clustering can comprise using random forest models. In some cases, clustering can comprise boosted tree models. In some cases, clustering can comprise using support vector machines. In some cases, clustering can comprise calculating one or more N-1 dimensional surfaces in N-dimensional space that partitions a dataset into clusters. In some cases, clustering can comprise distribution-based clustering. In some cases, clustering can comprise fitting a plurality of prior distributions over the data distributed in N-dimensional space. In some cases, clustering can comprise using density-based clustering. In some cases, clustering can comprise using fuzzy clustering. In some cases, clustering can comprise computing probability values of a data point belonging to a cluster. In some cases, clustering can comprise using constraints. In some cases, clustering can comprise using supervised learning. In some embodiments, clustering can comprise using unsupervised learning.

In some cases, clustering can comprise grouping molecules based on similarity. In some cases, clustering can comprise grouping molecules based on quantitative similarity. In some cases, clustering can comprise grouping molecules based on one or more features of each molecule. In some cases, clustering can comprise grouping molecules based on one or more labels of each molecule. In some cases, clustering can comprise grouping molecules based on Euclidean coordinates in a numerical representation of molecules. In some cases, clustering can comprise grouping molecules based on protein structural groups or functional groups (e.g., protein structures, substructures, or functional groups from protein databases such as Protein Data Bank or CATH Protein Structure Classification database). In some cases, a protein structural group or functional group may comprise protein primary structure, secondary structure, tertiary structure, or quaternary structure. In some cases, a protein structural group or functional group may be based at least partially on alpha helices, beta sheets, relative distribution of amino acids with different properties (e.g., aliphatic, aromatic, hydrophilic, acidic, basic, etc.), structural families (e.g., TIM barrel and beta barrel fold), protein domains (e.g., Death effector domain). In some cases, a protein structural group or functional group may be based at least partially on functional or spatial properties (e.g., functional groups-group of immune globulins, cytokines, cytoskeletal biomolecules, etc.).

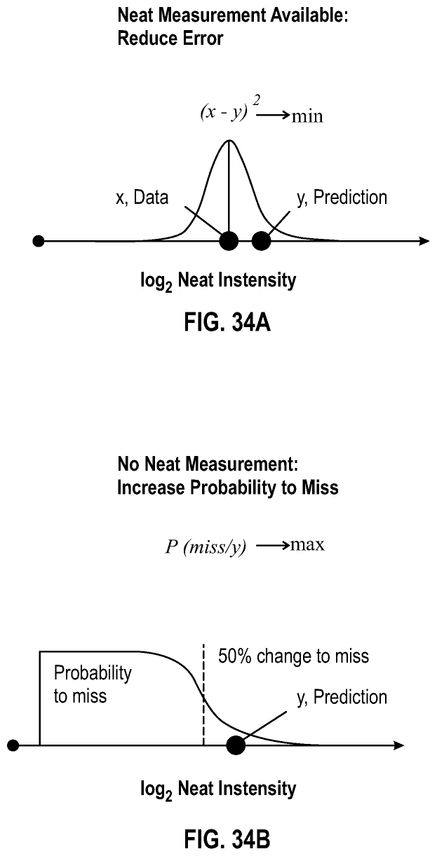

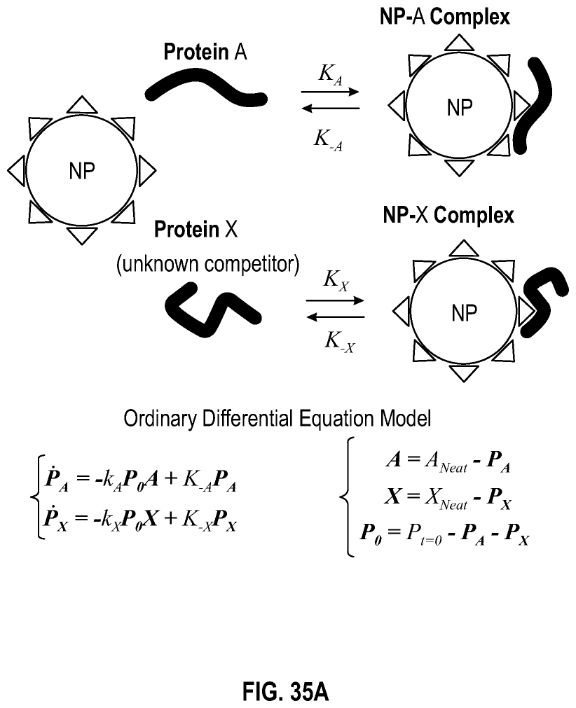

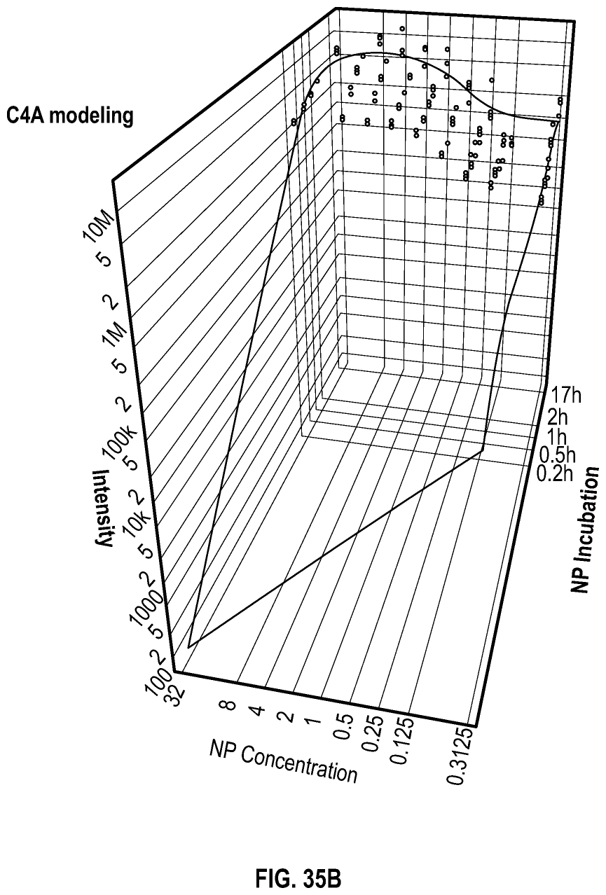

In some embodiments, the machine learning algorithm can be at least partially optimized using a regression loss function when the input dataset comprises a quantity in the quantities and a reference quantity in the reference quantities. For example, FIG. 34A shows an example regression loss function that can be used to optimize the machine learning algorithm when training data has the quantity and the reference quantity that can be used for direct comparison.

In some embodiments, the machine learning algorithm can incorporate the fact that a reference quantity is not contained in the reference quantities of the input data by implementing a specific loss function. In some embodiments, the machine learning algorithm can be at least partially trained using a logistic loss function when the input dataset does not comprise either a quantity in the quantities or a reference quantity in the reference quantities. For example, FIG. 34B shows an example logistic loss function that can be used to optimize the machine learning algorithm when training data has the quantity but not the reference quantity. The logistic loss function can be configured to increase the probability to miss the detection of the reference quantity (such as when the reference quantity is below the limit of detection measured using at least the second condition).

When trained, the machine learning algorithm can generate an output value that can be a normalization value for adjusting the quantities of the plurality of molecules. The normalization value can be the difference between a quantity and a reference quantity. The normalization value can be a ratio between a quantity and a reference quantity. When trained, the machine learning algorithm can generate an output value that is an adjusted quantity.

A trained machine learning algorithm can be used to generate an adjusted quantity of a molecule at a reference condition using a measured quantity of the molecule at another condition. The molecule can be in the training dataset. Alternatively, the molecule can be not in the training dataset. This is possible, for example, in situations where the molecule's derivative information is “close to” or “in between” the derivative information of other molecules in the training dataset. The derivative information of one molecule (e.g., changes in quantities with respect to one or more physicochemical parameters) can be expressed as a point in a coordinate system comprising the one or more physicochemical parameters. For instance, the coordinate system can comprise dimensions such as {∂Q/∂X₁, ∂Q/∂X₂, ∂Q/∂X₃, . . . ∂Q/∂Xₙ}, where ∂Q/∂X₁ can be the derivative information with respect to a first parameter (e.g., P/NP ratio), ∂Q/∂X₂ can be the derivative information with respect to a second parameter (e.g., incubation time), and so forth. The derivative information measured from a molecule can then be visualized as a point in this coordinate system, and the derivative information of all molecules in the training dataset can be visualized as a cloud of points in the coordinate system. Even when a molecule was not explicitly in the training dataset, if its coordinate in the coordinate system between the cloud of points, the machine learning algorithm may generate a useful normalization value. The magnitude of the predicted quantity can be below a detection limit of the method or device used to generate the machine learning model. In some embodiments, a magnitude of the predicted quantity is below a detection limit of the method or device used to generate the input dataset. For example, FIG. 33B shows a normalization value output by the machine learning algorithm that is used to adjust the quantity of the molecule below the detection limit of a mass spectrometer used to generate the training dataset.

A trained machine learning algorithm can be fine-tuned with additional datasets. A new input dataset can be provided, wherein the input dataset comprises features obtained for molecules in a condition different from the conditions in the initial training dataset. The new input dataset can comprise molecules in common with the initial training dataset, or no molecules in common. The new input dataset may be based on a different type of sample compared to the initial training dataset. As a non-limiting example, the machine learning algorithm may be initially trained using quantitative measurements under specific mass spectrometry conditions, and then later undergo additional training using a smaller subset of data obtained under different mass spectrometry conditions to account for changes in noise and/or deviations.

Adjusted quantities of the molecules can be more accurate or closer to the actual quantities in a sample, compared to the initially measured quantities. The adjusted quantities can be on average more accurate by at least 1, 2, 3, 4, 5, 6, 7, 8, 9, 10, 15, 20, 25, 30, 35, 40, 45, or 50 percent. The adjusted quantities can be more accurate by at most 1, 2, 3, 4, 5, 6, 7, 8, 9, 10, 15, 20, 25, 30, 35, 40, 45, or 50 percent. In some embodiments, the adjusted quantities are on average at least 10 percent more accurate. In some embodiments, the adjusted quantities are on average at least 20 percent more accurate. The average can be a mean or a median.

A coefficient of determination between the adjusted quantities of the plurality of molecules and the reference quantities of the pluralities of reference molecules can be at least 0.7, 0.8, 0.85, 0.9, 0.91, 0.92, 0.93, 0.94, 0.95, 0.96, 0.97, 0.98, or 0.99, when the plurality of molecules and the plurality of reference molecules are the same and the coefficient of determination is measured with a k-fold cross validation, wherein k is an integer greater than 1. A coefficient of determination between the adjusted quantities of the plurality of molecules and the reference quantities of the pluralities of reference molecules can be at most 0.7, 0.8, 0.85, 0.9, 0.91, 0.92, 0.93, 0.94, 0.95, 0.96, 0.97, 0.98, or 0.99, when the plurality of molecules and the plurality of reference molecules are the same and the coefficient of determination is measured with a k-fold cross validation, wherein k is an integer greater than 1. The k can be at least 2, 3, 4, 5, 6, 7, 8, 9, 10, 20, 30, 40, 50, 60, 70, 80, 90, or 100. The k can be at most 2, 3, 4, 5, 6, 7, 8, 9, 10, 20, 30, 40, 50, 60, 70, 80, 90, or 100.

A first coefficient of determination between the adjusted quantities of the plurality of molecules and the reference quantities of the pluralities of reference molecules can be greater than a second coefficient of determination between the quantities of the plurality of molecules and the reference quantities of the plurality of reference molecules by at least 0.01, 0.02, 0.03, 0.04, 0.05, 0.06, 0.07, 0.08, 0.09, 0.1, 0.2, 0.3, 0.4, or 0.5 when the plurality of molecules and the plurality of reference molecules are the same and the coefficient of determination is measured with a k-fold cross validation, wherein k is an integer greater than 1. A first coefficient of determination between the adjusted quantities of the plurality of molecules and the reference quantities of the pluralities of reference molecules can be greater than a second coefficient of determination between the quantities of the plurality of molecules and the reference quantities of the plurality of reference molecules by at most 0.01, 0.02, 0.03, 0.04, 0.05, 0.06, 0.07, 0.08, 0.09, 0.1, 0.2, 0.3, 0.4, or 0.5 when the plurality of molecules and the plurality of reference molecules are the same and the coefficient of determination is measured with a k-fold cross validation, wherein k is an integer greater than 1. The k can be at least 2, 3, 4, 5, 6, 7, 8, 9, 10, 20, 30, 40, 50, 60, 70, 80, 90, or 100. The k can be at most 2, 3, 4, 5, 6, 7, 8, 9, 10, 20, 30, 40, 50, 60, 70, 80, 90, or 100.

A mean absolute error (MAE) between the adjusted quantities of the plurality of molecules and the reference quantities of the pluralities of molecules can be at most 1, 2, 3, 4, 5, 6, 7, 8, 9, 10, 15, 20, 25, or 30 percent of the standard deviation of the reference quantities when the plurality of molecules and the plurality of reference molecules are the same and the MAE is measured with a k-fold cross validation, wherein k is an integer greater than 1. A mean absolute error (MAE) between the adjusted quantities of the plurality of molecules and the reference quantities of the pluralities of molecules can be at least 1, 2, 3, 4, 5, 6, 7, 8, 9, 10, 15, 20, 25, or 30 percent of the standard deviation of the reference quantities when the plurality of molecules and the plurality of reference molecules are the same and the MAE is measured with a k-fold cross validation, wherein k is an integer greater than 1. The k can be at least 2, 3, 4, 5, 6, 7, 8, 9, 10, 20, 30, 40, 50, 60, 70, 80, 90, or 100. The k can be at most 2, 3, 4, 5, 6, 7, 8, 9, 10, 20, 30, 40, 50, 60, 70, 80, 90, or 100.

A first mean absolute error (MAE) between the predicted quantities of the plurality of molecules and the reference quantities of the pluralities of molecules can be less than a second MAE between the quantities of the plurality of molecules and the reference quantities of the plurality of molecules by at least 1, 2, 3, 4, 5, 6, 7, 8, 9, 10, 15, 20, 25, or 30 percent of the standard deviation of the reference quantities when the plurality of molecules and the plurality of reference molecules are the same and the MAE is measured with a k-fold cross validation, wherein k is an integer greater than 1. A first mean absolute error (MAE) between the predicted quantities of the plurality of molecules and the reference quantities of the pluralities of molecules can be less than a second MAE between the quantities of the plurality of molecules and the reference quantities of the plurality of molecules by at most 1, 2, 3, 4, 5, 6, 7, 8, 9, 10, 15, 20, 25, or 30 percent of the standard deviation of the reference quantities when the plurality of molecules and the plurality of reference molecules are the same and the MAE is measured with a k-fold cross validation, wherein k is an integer greater than 1. The k can be at least 2, 3, 4, 5, 6, 7, 8, 9, 10, 20, 30, 40, 50, 60, 70, 80, 90, or 100. The k can be at most 2, 3, 4, 5, 6, 7, 8, 9, 10, 20, 30, 40, 50, 60, 70, 80, 90, or 100.

### Biological Sample

The present disclosure includes systems and methods for measuring and adjusting quantities of a biomolecule in a biological sample. In some cases, a biological sample may comprise a cell or be cell-free sample. In some cases, a biological sample may comprise a biofluid, such as blood, serum, plasma, urine, or cerebrospinal fluid (CSF). In some cases, a biofluid may be a fluidized solid, for example a tissue homogenate, or a fluid extracted from a biological sample. A biological sample may be, for example, a tissue sample or a fine needle aspiration (FNA) sample. A biological sample may be a cell culture sample. For example, a biofluid may be a fluidized cell culture extract. In some cases, a biological sample may be obtained from a subject. In some cases, the subject may be a human or a non-human. In some cases, the subject may be a plant, a fungus, or an archaeon. In some cases, a biological sample can contain a plurality of proteins or proteomic data, which may be analyzed after adsorption or binding of proteins to the surfaces of the various sensor element (e.g., particle) types in a panel and subsequent digestion of protein coronas.

In some cases, a biological sample may comprise plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, cell culture media, or any combination thereof. In some cases, a biological sample may comprise multiple biological samples (e.g., pooled plasma from multiple subjects, or multiple tissue samples from a single subject). In some cases, a biological sample may comprise a single type of biofluid or biomaterial from a single source.

In some cases, a biological sample may be diluted or pre-treated. In some cases, a biological sample may undergo depletion (e.g., the biological sample comprises serum) prior to or following contact with a surface disclosed herein. In some cases, a biological sample may undergo physical (e.g., homogenization or sonication) or chemical treatment prior to or following contact with a surface disclosed herein. In some cases, a biological sample may be diluted prior to or following contact with a surface disclosed herein. In some cases, a dilution medium may comprise buffer or salts, or be purified water (e.g., distilled water). In some cases, a biological sample may be provided in a plurality of partitions, wherein each partition may undergo different degrees of dilution. In some cases, a biological sample may comprise may undergo at least about 1.1-fold, 1.2-fold, 1.3-fold, 1.4-fold, 1.5-fold, 2-fold, 3-fold, 4-fold, 5-fold, 6-fold, 8-fold, 10-fold, 12-fold, 15-fold, 20-fold, 30-fold, 40-fold, 50-fold, 75-fold, 100-fold, 200-fold, 500-fold, or 1000-fold dilution.

In some cases, the biological sample may comprise a plurality of biomolecules. In some cases, a plurality of biomolecules may comprise polyamino acids. In some cases, the polyamino acids comprise peptides, proteins, or a combination thereof. In some cases, the plurality of biomolecules may comprise nucleic acids, carbohydrates, polyamino acids, or any combination thereof. A biological sample may comprise a member of any class of biomolecules, where “classes” may refer to any named category that defines a group of biomolecules having a common characteristic (e.g., proteins, nucleic acids, carbohydrates).

The one or more features can be obtained from a sample comprising the plurality of molecules. The sample can comprise at most about 1000, 100, 10, 1, 0.1, 0.01, or 0.001 nanograms of biomolecules. The sample can comprise at most about 1000, 100, 10, 1, 0.1, 0.01, or 0.001 nanograms of biomolecules per mL of the sample. The sample can comprise from at most about 1000, 100, 10, or 1 cell. The sample can comprise at most about 1000, 100, 10, 1, 0.1, 0.01, or 0.001 microliters. The sample can comprise a complex biological sample. The sample can comprise a plurality of biomolecules. The sample can comprise a plurality of proteins. The sample can comprise a plurality of proteoforms. The plurality of proteoforms can comprise a splicing variant. The plurality of proteoforms can comprise an allelic variant. The plurality of proteoforms can comprise a post-translational cleavage variant. The plurality of proteoforms can comprise a phosphorylated variant. The sample can comprise a plurality of lipids. The sample can comprise a plurality of nucleic acids. The sample can comprise a plurality of metabolites. The sample can comprise a plurality of secreted molecules.

### Proteomic Analysis

As used herein, “proteomic analysis”, “protein analysis”, and the like, may refer to any system or method for analyzing proteins in a sample, including the systems and methods disclosed herein. The present disclosure systems and methods for assaying using one or more surface. In some cases, a surface may comprise a surface of a high surface-area material, such as nanoparticles, particles, or porous materials. As used herein, a “surface” may refer to a surface for assaying polyamino acids. When a particle composition, physical property, or use thereof is described herein, it shall be understood that a surface of the particle may comprise the same composition, the same physical property, or the same use thereof, in some cases. Similarly, when a surface composition, physical property, or use thereof is described herein, it shall be understood that a particle may comprise the surface to comprise the same composition, the same physical property, or the same use thereof.

Materials for particles and surfaces may include metals, polymers, magnetic materials, and lipids. In some cases, magnetic particles may be iron oxide particles. Examples of metallic materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron, cadmium, or any alloys thereof. In some cases, a particle disclosed herein may be a magnetic particle, such as a superparamagnetic iron oxide nanoparticle (SPION). In some cases, a magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material).

The present disclosure describes panels of particles or surfaces. In some cases, a panel may comprise more than one distinct surface types. Panels described herein can vary in the number of surface types and the diversity of surface types in a single panel. For example, surfaces in a panel may vary based on size, polydispersity, shape and morphology, surface charge, surface chemistry and functionalization, and base material. In some cases, panels may be incubated with a sample to be analyzed for polyamino acids, polyamino acid concentrations, nucleic acids, nucleic acid concentrations, or any combination thereof. In some cases, polyamino acids in the sample adsorb to distinct surfaces to form one or more adsorption layers of biomolecules. The identity of the biomolecules and concentrations thereof in the one or more adsorption layers may depend on the physical properties of the distinct surfaces and the physical properties of the biomolecules. Thus, each surface type in a panel may have differently adsorbed biomolecules due to adsorbing a different set of biomolecules, different concentrations of a particular biomolecules, or a combination thereof. Each surface type in a panel may have mutually exclusive adsorbed biomolecules or may have overlapping adsorbed biomolecules.

In some cases, panels disclosed herein can be used to identify the number of distinct biomolecules disclosed herein over a wide dynamic range in a given biological sample. For example, a panel may enrich a subset of biomolecules in a sample, which can be identified over a wide dynamic range at which the biomolecules are present in a sample (e.g., a plasma sample). In some cases, the enriching may be selective—e.g., biomolecules in the subset may be enriched but biomolecules outside of the subset may not enriched and/or be depleted. In some cases, the subset may comprise proteins having different post-translational modifications. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some cases, the panel including any number of distinct particle types disclosed herein, enriches and identifies a single protein or protein group by binding different domains, sequences, or epitopes of the protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group. In some cases, a panel including any number of distinct particle types disclosed herein, may enrich and identify biomolecules over a dynamic range of at least 5, 6, 7, 8, 9, 10, 15, or 20 magnitudes. In some cases, a panel including any number of distinct particle types disclosed herein, may enrich and identify biomolecules over a dynamic range of at most 5, 6, 7, 8, 9, 10, 15, or 20 magnitudes.

A panel can have more than one surface type. Increasing the number of surface types in a panel can be a method for increasing the number of proteins that can be identified in a given sample.

A particle or surface may comprise a polymer. The polymer may constitute a core material (e.g., the core of a particle may comprise a particle), a layer (e.g., a particle may comprise a layer of a polymer disposed between its core and its shell), a shell material (e.g., the surface of the particle may be coated with a polymer), or any combination thereof. Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). The polymer may comprise a cross link. A plurality of polymers in a particle may be phase separated or may comprise a degree of phase separation.

Examples of lipids that can be used to form the particles or surfaces of the present disclosure include cationic, anionic, and neutrally charged lipids. For example, particles and/or surfaces can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, cholesterol, and any combination thereof.

A particle panel may comprise a combination of particles with silica and polymer surfaces. For example, a particle panel may comprise a SPION coated with a thin layer of silica, a SPION coated with poly(dimethyl aminopropyl methacrylamide) (PDMAPMA), and a SPION coated with poly(ethylene glycol) (PEG). A particle panel consistent with the present disclosure could also comprise two or more particles selected from the group consisting of silica coated SPION, an N-(3-Trimethoxysilylpropyl) diethylenetriamine coated SPION, a PDMAPMA coated SPION, a carboxyl-functionalized polyacrylic acid coated SPION, an amino surface functionalized SPION, a polystyrene carboxyl functionalized SPION, a silica particle, and a dextran coated SPION. A particle panel consistent with the present disclosure may also comprise two or more particles selected from the group consisting of a surfactant free carboxylate microparticle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N-[3-(Dimethylamino) propyl] methacrylamide-co-[2-(methacryloyloxy)ethyl]dimethyl-(3-sulfopropyl) ammonium hydroxide, P (DMAPMA-co-SBMA) coated particle. A particle panel consistent with the present disclosure may comprise silica-coated particles, N-(3-Trimethoxysilylpropyl) diethylenetriamine coated particles, poly(N-(3-(dimethylamino) propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof.

A particle panel consistent with the present disclosure may comprise a silica functionalized particle, an amine functionalized particle, a silicon alkoxide functionalized particle, a carboxylate functionalized particle, and a benzyl or phenyl functionalized particle. A particle panel consistent with the present disclosure may comprise a silica functionalized particle, an amine functionalized particle, a silicon alkoxide functionalized particle, a polystyrene functionalized particle, and a saccharide functionalized particle. A particle panel consistent with the present disclosure may comprise a silica functionalized particle, an N-(3-Trimethoxysilylpropyl) diethylenetriamine functionalized particle, a PDMAPMA functionalized particle, a dextran functionalized particle, and a polystyrene carboxyl functionalized particle. A particle panel consistent with the present disclosure may comprise 5 particles including a silica functionalized particle, an amine functionalized particle, a silicon alkoxide functionalized particle. In some embodiments, the particle panel comprises a silica functionalized particle, an amine functionalized particle, and a carboxyl functionalized particle.

Distinct surfaces or distinct particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group. A small molecule functionalization may comprise a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle.

A particle may comprise a single surface such as a specific small molecule, or a plurality of surface functionalizations, such as a plurality of different small molecules. Surface functionalization can influence the composition of a particle's biomolecule corona. Such surface functionalization can include small molecule functionalization or macromolecular functionalization. A surface functionalization may be coupled to a particle material such as a polymer, metal, metal oxide, inorganic oxide (e.g., silicon dioxide), or another surface functionalization.

A surface functionalization may comprise a small molecule functionalization, a macromolecular functionalization, or a combination of two or more such functionalizations. In some cases, a macromolecular functionalization may comprise a biomacromolecule, such as a protein or a polynucleotide (e.g., a 100-mer DNA molecule). A macromolecular functionalization may comprise a protein, polynucleotide, or polysaccharide, or may be comparable in size to any of the aforementioned classes of species. In some cases, a surface functionalization may comprise an ionizable moiety. In some cases, a surface functionalization may comprise pKa of at least about 1, 2, 3, 4, 5, 6, 7, 8, 9, 10, 11, 12, 13, or 14. In some cases, a surface functionalization may comprise pKa of at most about 1, 2, 3, 4, 5, 6, 7, 8, 9, 10, 11, 12, 13, or 14. In some cases, a small molecule functionalization may comprise a small organic molecule such as an alcohol (e.g., octanol), an amine, an alkane, an alkene, an alkyne, a heterocycle (e.g., a piperidinyl group), a heteroaromatic group, a thiol, a carboxylate, a carbonyl, an amide, an ester, a thioester, a carbonate, a thiocarbonate, a carbamate, a thiocarbamate, a urea, a thiourea, a halogen, a sulfate, a phosphate, a monosaccharide, a disaccharide, a lipid, or any combination thereof. For example, a small molecule functionalization may comprise a phosphate sugar, a sugar acid, or a sulfurylated sugar.

In some cases, a macromolecular functionalization may comprise a specific form of attachment to a particle. In some cases, a macromolecule may be tethered to a particle via a linker. In some cases, the linker may hold the macromolecule close to the particle, thereby restricting its motion and reorientation relative to the particle, or may extend the macromolecule away from the particle. In some cases, the linker may be rigid (e.g., a polyolefin linker) or flexible (e.g., a nucleic acid linker). In some cases, a linker may be at least about 0.5, 1, 2, 3, 4, 5, 6, 7, 8, 9, 10, 15, 20, 25, or 30 nm in length. In some cases, a linker may be at most about 0.5, 1, 2, 3, 4, 5, 6, 7, 8, 9, 10, 15, 20, 25, or 30 nm in length. As such, a surface functionalization on a particle may project beyond a primary corona associated with the particle. In some cases, a surface functionalization may also be situated beneath or within a biomolecule corona that forms on the particle surface. In some cases, a macromolecule may be tethered at a specific location, such as at a protein's C-terminus, or may be tethered at a number of possible sites. For example, a peptide may be covalent attached to a particle via any of its surface exposed lysine residues.

In some cases, a particle may be contacted with a biological sample (e.g., a biofluid) to form a biomolecule corona. In some cases, a biomolecule corona may comprise at least two biomolecules that do not share a common binding motif. The particle and biomolecule corona may be separated from the biological sample, for example by centrifugation, magnetic separation, filtration, or gravitational separation. The particle types and biomolecule corona may be separated from the biological sample using a number of separation techniques. Non-limiting examples of separation techniques include comprises magnetic separation, column-based separation, filtration, spin column-based separation, centrifugation, ultracentrifugation, density or gradient-based centrifugation, gravitational separation, or any combination thereof. A protein corona analysis may be performed on the separated particle and biomolecule corona. A protein corona analysis may comprise identifying one or more proteins in the biomolecule corona, for example by mass spectrometry. In some cases, a single particle type may be contacted with a biological sample. In some cases, a plurality of particle types may be contacted to a biological sample. In some cases, the plurality of particle types may be combined and contacted to the biological sample in a single sample volume. In some cases, the plurality of particle types may be sequentially contacted to a biological sample and separated from the biological sample prior to contacting a subsequent particle type to the biological sample. In some cases, adsorbed biomolecules on the particle may have compressed (e.g., smaller) dynamic range compared to a given original biological sample.

In some cases, the particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. In some cases, the biomolecule corona on each particle type used for serial interrogation of a sample may be analyzed by protein corona analysis. The biomolecule content of the supernatant may be analyzed following serial interrogation with one or more particle types.

In some cases, a method of the present disclosure may identify a large number of unique biomolecules (e.g., proteins) in a biological sample (e.g., a biofluid). In some cases, a surface disclosed herein may be incubated with a biological sample to adsorb at least 1000, 2000, 3000, 4000, 5000, 6000, 7000, 8000, 9000, or 10000 unique biomolecules. In some cases, a surface disclosed herein may be incubated with a biological sample to adsorb at most 1000, 2000, 3000, 4000, 5000, 6000, 7000, 8000, 9000, or 10000 unique biomolecules. In some cases, a surface disclosed herein may be incubated with a biological sample to adsorb at least 1000, 2000, 3000, 4000, 5000, 6000, 7000, 8000, 9000, or 10000 unique biomolecule groups. In some cases, a surface disclosed herein may be incubated with a biological sample to adsorb at most 1000, 2000, 3000, 4000, 5000, 6000, 7000, 8000, 9000, or 10000 unique biomolecule groups. In some cases, several different types of surfaces can be used, separately or in combination, to identify large numbers of proteins in a particular biological sample. In other words, surfaces can be multiplexed in order to bind and identify large numbers of biomolecules in a biological sample.

In some cases, a method of the present disclosure may identify a large number of unique proteoforms in a biological sample. In some cases, a method may identify at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a method may identify at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb at least 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb at most 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, several different types of surfaces can be used, separately or in combination, to identify large numbers of proteins in a particular biological sample. In other words, surfaces can be multiplexed in order to bind and identify large numbers of biomolecules in a biological sample.

Biomolecules collected on particles may be subjected to further analysis. In some cases, a method may comprise collecting a biomolecule corona or a subset of biomolecules from a biomolecule corona. In some cases, the collected biomolecule corona or the collected subset of biomolecules from the biomolecule corona may be subjected to further particle-based analysis (e.g., particle adsorption). In some cases, the collected biomolecule corona or the collected subset of biomolecules from the biomolecule corona may be purified or fractionated (e.g., by a chromatographic method). In some cases, the collected biomolecule corona or the collected subset of biomolecules from the biomolecule corona may be analyzed (e.g., by mass spectrometry).

In some cases, the panels disclosed herein can be used to identify a number of proteins, peptides, protein groups, or protein classes using a protein analysis workflow described herein (e.g., a protein corona analysis workflow). In some cases, the panels disclosed herein can be used to identify at least 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 unique proteins. In some cases, the panels disclosed herein can be used to identify at most 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 unique proteins. In some cases, the panels disclosed herein can be used to identify at least 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 protein groups. In some cases, the panels disclosed herein can be used to identify at most 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 protein groups. In some cases, the panels disclosed herein can be used to identify at least 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, or 1000000 peptides. In some cases, the panels disclosed herein can be used to identify at most 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, or 1000000 peptides. In some cases, a peptide may be a tryptic peptide. In some cases, a peptide may be a semi-tryptic peptide. In some cases, protein analysis may comprise contacting a sample to distinct surface types (e.g., a particle panel), forming adsorbed biomolecule layers on the distinct surface types, and identifying the biomolecules in the adsorbed biomolecule layers (e.g., by mass spectrometry). Feature intensities, as disclosed herein, may refer to the intensity of a discrete spike (“feature”) seen on a plot of mass to charge ratio versus intensity from a mass spectrometry run of a sample. In some cases, these features can correspond to variably ionized fragments of peptides and/or proteins. In some cases, using the data analysis methods described herein, feature intensities can be sorted into protein groups. In some cases, protein groups may refer to two or more proteins that are identified by a shared peptide sequence. In some cases, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). In some cases, if the peptide sequence is unique to a single protein (Protein 1), a protein group could be the “ZZX” protein group having one member (Protein 1). In some cases, each protein group can be supported by more than one peptide sequence. In some cases, protein detected or identified according to the instant disclosure can refer to a distinct protein detected in the sample (e.g., distinct relative other proteins detected using mass spectrometry). In some cases, analysis of proteins present in distinct coronas corresponding to the distinct surface types in a panel yields a high number of feature intensities. In some cases, this number decreases as feature intensities are processed into distinct peptides, further decreases as distinct peptides are processed into distinct proteins, and further decreases as peptides are grouped into protein groups (two or more proteins that share a distinct peptide sequence).

In some cases, the methods disclosed herein include isolating one or more particle types from a sample or from more than one sample (e.g., a biological sample or a serially interrogated sample). The particle types can be rapidly isolated or separated from the sample using a magnet. Moreover, multiple samples that are spatially isolated can be processed in parallel. In some cases, the methods disclosed herein provide for isolating or separating a particle type from unbound protein in a sample. In some cases, a particle type may be separated by a variety of means, including but not limited to magnetic separation, centrifugation, filtration, or gravitational separation. In some cases, particle panels may be incubated with a plurality of spatially isolated samples, wherein each spatially isolated sample is in a well in a well plate (e.g., a 96-well plate). In some cases, the particle in each of the wells of the well plate can be separated from unbound protein present in the spatially isolated samples by placing the entire plate on a magnet. In some cases, this simultaneously pulls down the superparamagnetic particles in the particle panel. In some cases, the supernatant in each sample can be removed to remove the unbound protein. In some cases, these steps (incubate, pull down) can be repeated to effectively wash the particles, thus removing residual background unbound protein that may be present in a sample.

In some cases, the systems and methods disclosed herein may also elucidate protein classes or interactions of the protein classes. In some cases, a protein class may comprise a set of proteins that share a common function (e.g., amine oxidases or proteins involved in angiogenesis); proteins that share common physiological, cellular, or subcellular localization (e.g., peroxisomal proteins or membrane proteins); proteins that share a common cofactor (e.g., heme or flavin proteins); proteins that correspond to a particular biological state (e.g., hypoxia related proteins); proteins containing a particular structural motif (e.g., a cupin fold); proteins that are functionally related (e.g., part of a same metabolic pathway); or proteins bearing a post-translational modification (e.g., ubiquitinated or citrullinated proteins). In some cases, a protein class may contain at least 2 proteins, 5 proteins, 10 proteins, 20 proteins, 40 proteins, 60 proteins, 80 proteins, 100 proteins, 150 proteins, 200 proteins, or more.

In some cases, the proteomic data of the biological sample can be identified, measured, and quantified using a number of different analytical techniques. For example, proteomic data can be generated using SDS-PAGE or any gel-based separation technique. In some cases, peptides and proteins can also be identified, measured, and quantified using an immunoassay, such as ELISA. In some cases, proteomic data can be identified, measured, and quantified using mass spectrometry, high performance liquid chromatography, LC-MS/MS, Edman Degradation, immunoaffinity techniques, and other protein separation techniques.

In some cases, an assay may comprise protein collection of particles, protein digestion, and mass spectrometric analysis (e.g., MS, LC-MS, LC-MS/MS). In some cases, the digestion may comprise chemical digestion, such as by cyanogen bromide or 2-Nitro-5-thiocyanatobenzoic acid (NTCB). In some cases, the digestion may comprise enzymatic digestion, such as by trypsin or pepsin. In some cases, the digestion may comprise enzymatic digestion by a plurality of proteases. In some cases, the digestion may comprise a protease selected from among the group consisting of trypsin, chymotrypsin, Glu C, Lys C, elastase, subtilisin, proteinase K, thrombin, factor X, Arg C, papaine, Asp N, thermolysine, pepsin, aspartyl protease, cathepsin D, zinc mealloprotease, glycoprotein endopeptidase, proline, aminopeptidase, prenyl protease, caspase, kex2 endoprotease, or any combination thereof. In some cases, the digestion may cleave peptides at random positions. In some cases, the digestion may cleave peptides at a specific position (e.g., at methionines) or sequence (e.g., glutamate-histidine-glutamate). In some cases, the digestion may enable similar proteins to be distinguished. For example, an assay may resolve 8 distinct proteins as a single protein group with a first digestion method, and as 8 separate proteins with distinct signals with a second digestion method. In some cases, the digestion may generate an average peptide fragment length of 8 to 15 amino acids. In some cases, the digestion may generate an average peptide fragment length of 12 to 18 amino acids. In some cases, the digestion may generate an average peptide fragment length of 15 to 25 amino acids. In some cases, the digestion may generate an average peptide fragment length of 20 to 30 amino acids. In some cases, the digestion may generate an average peptide fragment length of 30 to 50 amino acids.

In some cases, an assay may rapidly generate and analyze proteomic data. In some cases, beginning with an input biological sample (e.g., a buccal or nasal smear, plasma, or tissue), a method of the present disclosure may generate and analyze proteomic data in less than about 1, 2, 3, 4, 5, 6, 7, 8, 12, 16, 20, 24, or 48 hours. In some cases, the analyzing may comprise identifying a protein group. In some cases, the analyzing may comprise identifying a protein class. In some cases, the analyzing may comprise quantifying an abundance of a biomolecule, a peptide, a protein, protein group, or a protein class. In some cases, the analyzing may comprise identifying a ratio of abundances of two biomolecules, peptides, proteins, protein groups, or protein classes. In some cases, the analyzing may comprise identifying a biological state.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl) diethylenetriamine coated SPION, a poly(N-(3-(dimethylamino) propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo (ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 μm in diameter, a silica amino functionalized microparticle of about 0.1-0.39 μm in diameter, a Jeffamine surface particle of about 0.1-0.39 μm in diameter, a polystyrene microparticle of about 2.0-2.9 μm in diameter, a silica particle, a carboxylated particle with an original coating of about 50 nm in diameter, a particle coated with a dextran based coating of about 0.13 μm in diameter, or a silica silanol coated particle with low acidity. In some cases, a particle may lack functionalized specific binding moieties for specific binding on its surface. In some cases, a particle may lack functionalized proteins for specific binding on its surface. In some cases, a surface functionalized particle does not comprise an antibody or a T cell receptor, a chimeric antigen receptor, a receptor protein, or a variant or fragment thereof. In some cases, the ratio between surface area and mass can be a determinant of a particle's properties. The particles disclosed herein can have surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. Small particles (e.g., with diameters of 50 nm or less) can have significantly higher surface area to mass ratios, stemming in part from the higher order dependence on diameter by mass than by surface area. In some cases (e.g., for small particles), the particles can have surface area to mass ratios of 200 to 1000 cm²/mg, 500 to 2000 cm²/mg, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg. A particle may comprise a wide array of physical properties. A physical property of a particle may include composition, size, surface charge, hydrophobicity, hydrophilicity, amphipathicity, surface functionality, surface topography, surface curvature, porosity, core material, shell material, shape, zeta potential, and any combination thereof. A particle may have a core-shell structure. In some cases, a core material may comprise metals, polymers, magnetic materials, paramagnetic materials, oxides, and/or lipids. In some cases, a shell material may comprise metals, polymers, magnetic materials, oxides, and/or lipids.

### Proteomic Information

The computer-implemented systems and methods disclosed herein can be used to adjust protein quantities included with, or used to infer, additional proteomic information. In some cases, proteomic information or data can refer to information about substances comprising a peptide and/or a protein component. In some cases, proteomic information may comprise primary structure information, secondary structure information, tertiary structure information, or quaternary information about the peptide or a protein. In some cases, proteomic information may comprise information about protein-ligand interactions, wherein a ligand may comprise any one of various biological molecules and substances that may be found in living organisms, such as, nucleotides, nucleic acids, amino acids, peptides, proteins, monosaccharides, polysaccharides, lipids, phospholipids, hormones, or any combination thereof.

In some cases, proteomic information may comprise information about a single cell, a tissue, an organ, a system of tissues and/or organs (such as cardiovascular, respiratory, digestive, or nervous systems), or an entire multicellular organism. In some cases, proteomic information may comprise information about an individual (e.g., an individual human being or an individual bacterium), or a population of individuals (e.g., human beings with diagnosed with cancer or a colony of bacteria). Proteomic information may comprise information from various forms of life, including forms of life from the Archaea, the Bacteria, the Eukarya, the Protozoa, the Chromista, the Plantae, the Fungi, or from the Animalia. In some cases, proteomic information may comprise information from viruses.

In some cases, proteomic information may comprise information relating exons and introns in the code of life. In some cases, proteomic information may comprise information regarding variations in the primary structure, variations in the secondary structure, variations in the tertiary structure, or variations in the quaternary structure of peptides and/or proteins. In some cases, proteomic information may comprise information regarding variations in the expression of exons, including alternative splicing variations, structural variations, or both. In some cases, proteomic information may comprise conformation information, post-translational modification information, chemical modification information (e.g., phosphorylation), cofactor (e.g., salts or other regulatory chemicals) association information, or substrate association information of peptides and/or proteins.

In some cases, proteomic information may comprise information related to various proteoforms in a sample. In some cases, a proteomic information may comprise information related to peptide variants, protein variants, or both. In some cases, a proteomic information may comprise information related to splicing variants, allelic variants, post-translation modification variants, or any combination thereof.

In some cases, splicing variant (in some cases also referred to as “alternative splicing” variants, “differential splicing” variants, or “alternative RNA splicing” variants) may refer to a protein that is expressed by an alternative splicing process. In some cases, an alternative splicing process may express one or more splicing variants from a set of exons via different combinations of exons. In some cases, a combination may comprise a different sequence of exons compared to another combination. In some cases, a combination may comprise a different subset of exons compared to another combination. In some cases, a splicing variant may comprise a reordered amino acid sequence of another splicing variant.

In some cases, an allelic variant may refer to a protein that is expressed from a gene comprising a mutation compared to a reference gene. In some cases, the reference gene may be the gene of a cell, an individual, or a population of individuals. In some cases, the mutation may be a base substitution, a base deletion, or a base insertion of a genetic sequence of the gene compared to a genetic reference of the reference gene. In some cases, an allelic variant may comprise an amino acid substitution in an amino acid sequence of another allelic variant.

In some cases, a post-translation modification may refer to a protein that is modified after expression. A protein may be modified by various enzymes. In some cases, an enzyme that can modify a protein may be a kinase, a protease, a ligase, a phosphatase, a transferase, a phosphotransferase, or any other enzyme for performing the any one of modifications disclosed herein.

In some cases, peptide variants or protein variants may comprise a post-translation modification. In some cases, the post-translational modification comprises acylation, alkylation, prenylation, flavination, amination, deamination, carboxylation, decarboxylation, nitrosylation, halogenation, sulfurylation, glutathionylation, oxidation, oxygenation, reduction, ubiquitination, SUMOylation, neddylation, myristoylation, palmitoylation, isoprenylation, farnesylation, geranylgeranylation, glypiation, glycosylphosphatidylinositol anchor formation, lipoylation, heme functionalization, phosphorylation, phosphopantetheinylation, retinylidene Schiff base formation, diphthamide formation, ethanolamine phosphoglycerol functionalization, hypusine formation, beta-Lysine addition, acetylation, formylation, methylation, amidation, amide bond formation, butyrylation, gamma-carboxylation, glycosylation, polysialylation, malonylation, hydroxylation, iodination, nucleotide addition, phosphate ester formation, phosphoramidate formation, adenylation, uridylylation, propionylation, pyroglutamate formation, gluthathionylation, sulfenylation, sulfinylation, sulfonylation, succinylation, sulfation, glycation, carbonylation, isopeptide bond formation, biotinylation, carbamylation, oxidation, pegylation, citrullination, deamidation, eliminylation, disulfide bond formation, proteolytic cleavage, isoaspartate formation, racemization, protein splicing, chaperon-assisted folding, or any combination thereof.

In some cases, proteomic information may be encoded as digital information. In some cases, the proteomic information may comprise one or more elements that represents the proteomic information. In some cases, an element may represent a primary structure information, secondary structure information, tertiary structure information, or quaternary information about a peptide or a protein. In some cases, an element may represent protein-ligand interactions for a peptide or a protein. In some cases, an element may represent a source of a peptide or protein (e.g., a specific cell, tissue, organ, organism, individual, or population of inidividuals). In some cases, an element may represent a type of proteoform. In some cases, an element may be a number, a vector, an array, or any other datatypes provided herein.

### Non-Specific Binding

A surface may bind biomolecules through variably selective adsorption (e.g., adsorption of biomolecules or biomolecule groups upon contacting the particle to a biological sample comprising the biomolecules or biomolecule groups, which adsorption is variably selective depending upon factors including e.g., physicochemical properties of the particle) or non-specific binding. Non-specific binding can refer to a class of binding interactions that exclude specific binding. Examples of specific binding may comprise protein-ligand binding interactions, antigen-antibody binding interactions, nucleic acid hybridizations, or a binding interaction between a template molecule and a target molecule wherein the template molecule provides a sequence or a 3D structure that favors the binding of a target molecule that comprise a complementary sequence or a complementary 3D structure, and disfavors the binding of a non-target molecule(s) that does not comprise the complementary sequence or the complementary 3D structure.

Non-specific binding may comprise one or a combination of a wide variety of chemical and physical interactions and effects. Non-specific binding may comprise electromagnetic forces, such as electrostatics interactions, London dispersion, Van der Waals interactions, or dipole-dipole interactions (e.g., between both permanent dipoles and induced dipoles). Non-specific binding may be mediated through covalent bonds, such as disulfide bridges. Non-specific binding may be mediated through hydrogen bonds. Non-specific binding may comprise solvophobic effects (e.g., hydrophobic effect), wherein one object is repelled by a solvent environment and is forced to the boundaries of the solvent, such as the surface of another object. Non-specific binding may comprise entropic effects, such as in depletion forces, or raising of the thermal energy above a critical solution temperature (e.g., a lower critical solution temperature). Non-specific binding may comprise kinetic effects, wherein one binding molecule may have faster binding kinetics than another binding molecule.

Non-specific binding may comprise a plurality of non-specific binding affinities for a plurality of targets (e.g., at least 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 20,000, 30,000, 40,000, 50,000 different targets adsorbed to a single particle). The plurality of targets may have similar non-specific binding affinities that are within about one, two, or three magnitudes (e.g., as measured by non-specific binding free energy, equilibrium constants, competitive adsorption, etc.). This may be contrasted with specific binding, which may comprise a higher binding affinity for a given target molecule than non-target molecules.

Biomolecules may adsorb onto a surface through non-specific binding on a surface at various densities. In some cases, biomolecules or proteins may adsorb at a density of at least about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm². In some cases, biomolecules or proteins may adsorb at a density of at most about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm².

Adsorbed biomolecules may comprise various types of proteins. In some cases, adsorbed proteins may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins. In some cases, adsorbed proteins may comprise at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins.

In some cases, proteins in a biological sample may comprise at least about 1, 2, 3, 4, 5, 6, 7, 8, 9, 10, 15, 20, 25, or 30 orders of magnitudes in concentration. In some cases, proteins in a biological sample may comprise at most about 1, 2, 3, 4, 5, 6, 7, 8, 9, 10, 15, 20, 25, or 30 orders of magnitudes in concentration.

### Definitions

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “minimize”, “maximize”, “optimize”, “reduce”, “decrease”, “increase”, and the like, when used in the context of training a machine learning algorithm, can refer to the process of adjusting one or more parameters of a machine learning algorithm such that the value of a loss function is adjusted towards a defined objective (e.g., minimizing a difference between a machine learning output and examples).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The following illustrative examples are representative of embodiments of the systems and methods described herein and are not meant to be limiting in any way.

### Example 1

Sample preparation steps, such as protein extraction, depletion, proteolytic digestion, or peptide fractionation, as well as the LC-MS/MS methods can affect identification and quantification of analytes in a sample. In some cases, plasma itself is not the biologically active specimen of interest, but is rather a surrogate for whole blood. For many biological studies, it is important to obtain the relative differences between proteins in a sample, and also the linearity of response of those proteins to a measurement technique. In silico adjustment of a readout to match more closely the in vivo composition of a sample can be useful to derive more accurate and biologically-relevant estimates on the sample composition.

NP-coronas can quantitatively compress the dynamic range of proteomes to measurable levels. This can provide an informative but compressed representation of the molecular composition (concentrations) of a sample. The physicochemical property and biological activity of the resulting NP-proteins conglomerate can be a function of the NP surface design and the molecular composition and concentrations of the biosample. A machine learning model can be trained, which can be constructed without any a priori assumptions on the underlying physics of corona formation mechanisms, to predict neat biosample composition from the NP dilution and time-course data.

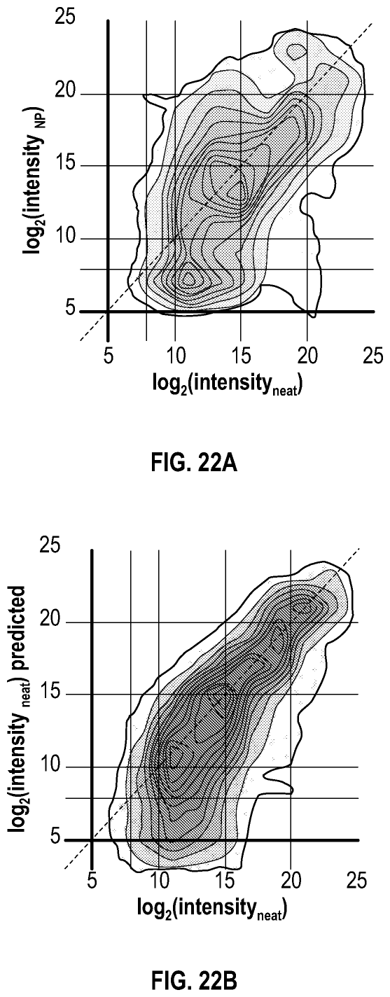

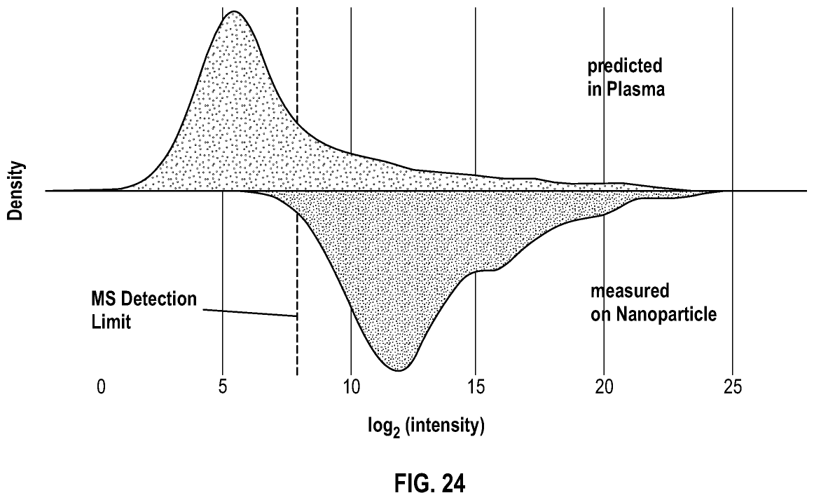

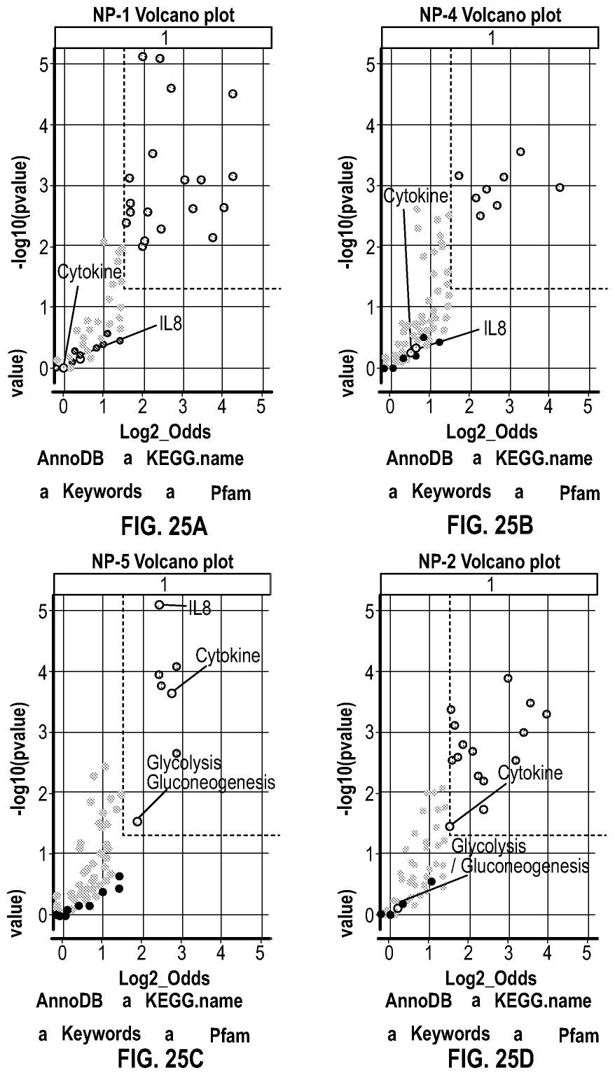

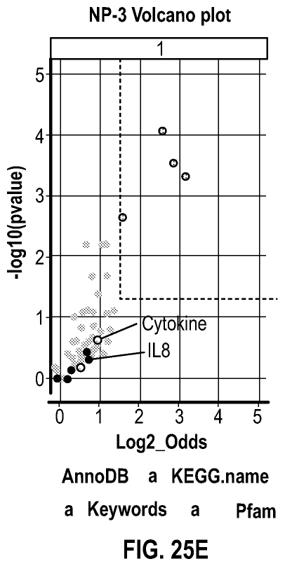

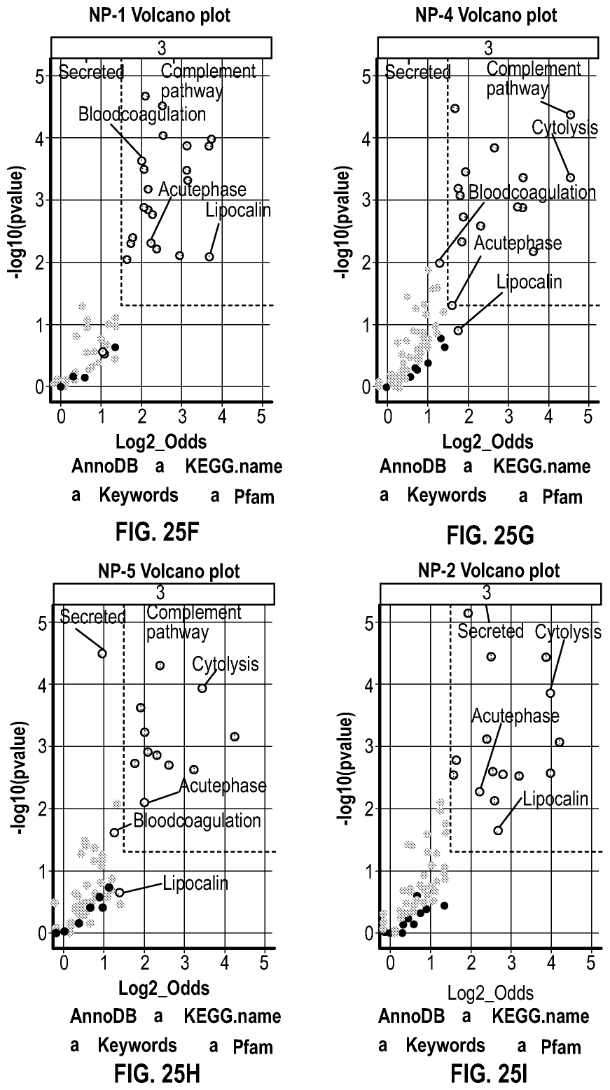

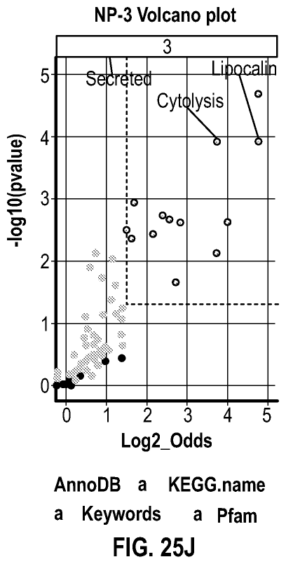

This example describes training a machine learning algorithm to predict protein concentration in the original plasma from protein intensity profiles obtained vi MS across NP conditions. The results show that, in comparison to the protein intensity measured in NP corona at 1 h, P/NP=10/1, the trained machine learning algorithm can predict adjusted values that are improved in correlation with the measured neat intensities from 58% to 79% and from 42% to 64% for protein concentrations reported in HPPP (as shown in FIGS. 22A-22B). Some of the reconstructed neat protein intensities fall below the detection threshold of the mass spectrometer. The detection threshold of the mass spectrometer is indicated as dotted grey lines in FIGS. 22A-22B. This demonstrates the ability to infer neat plasma compositions beyond the dynamic range capacity of conventional scalable plasma proteomics workflow (FIG. 24). These results demonstrate the feasibility of combining ML with quantitative deep NP workflows to protein coronas and reconstruct the low-abundance fraction of human plasma proteome quantitatively and accurately.

### Nanoparticle Engineering and Protein Corona Investigation

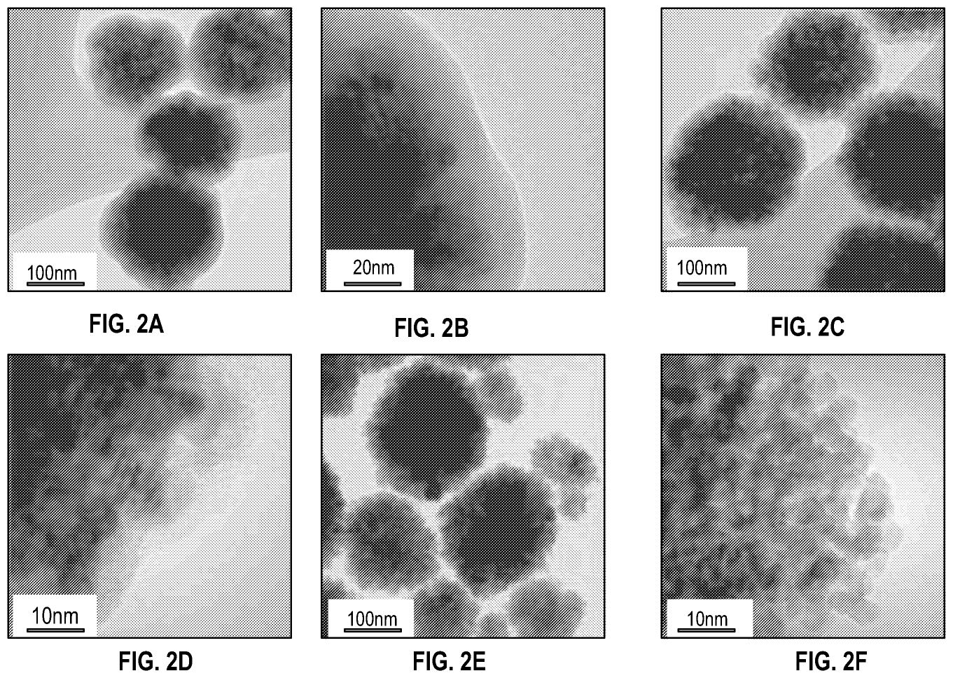

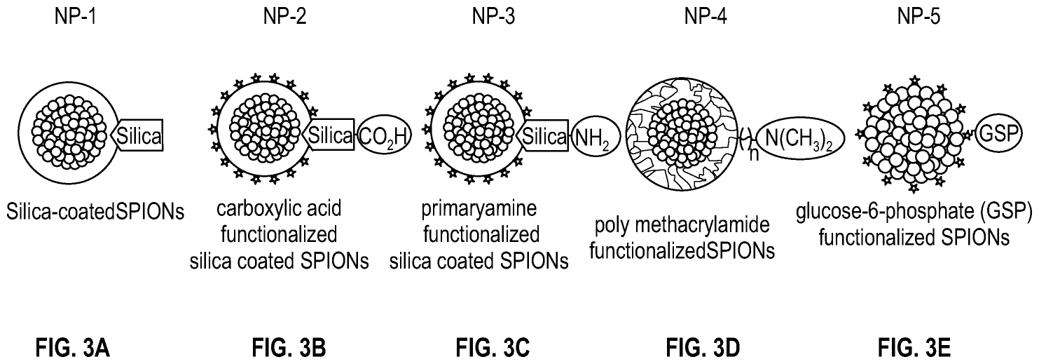

To investigate nano-bio interactions and NP corona formation kinetics, a series of distinct NPs were engineered, characterized, and compared. They were subjected to an automated high throughput deep proteome profiling workflow (FIG. 1A). Three NP classes with different surface chemical moieties (FIGS. 2A-2F and FIGS. 3A-3E) were compared. Those three NP classes include: (1) silica-coated SPIONs with core-shell structure and installed carboxylic acid-functionalized silane (NP-2), primary amine-functionalized silane (NP-3), and unfunctionalized surface (NP-1), representing three different types of surfaces of silica-coated SPIONs; (2) SPIONs coated with a polymethacrylamide (NP-4); and (3) glucose-6-phosphate (GSP)-functionalized SPIONs (NP-5). Importantly, the encapsulated SPIONs common to all five types of NPs enabled precise and scalable integration into fully automated, magnet-assisted end-to-end sample preparation, from corona formation to protein and peptide isolation for downstream proteome analysis.

Transmission electron microscopy (TEM), X-ray Photoelectron Spectroscopy (XPS), dynamic light scattering (DLS), and zeta potential measurements were performed to characterize the physiochemical properties of these NPs and to confirm successful synthesis of all five target NPs shown in FIGS. 3A-3E. TEM images of NP-1, NP-2, and NP-3 confirmed a core-shell structure with an average silica shell thickness of ˜14 nm (FIGS. 2A-2F). TEM images of NP-4 and NP-5 showed that amorphous polymer layers and small SPIONs (˜5-10 nm) are the predominant features present at the surface of these samples, respectively.

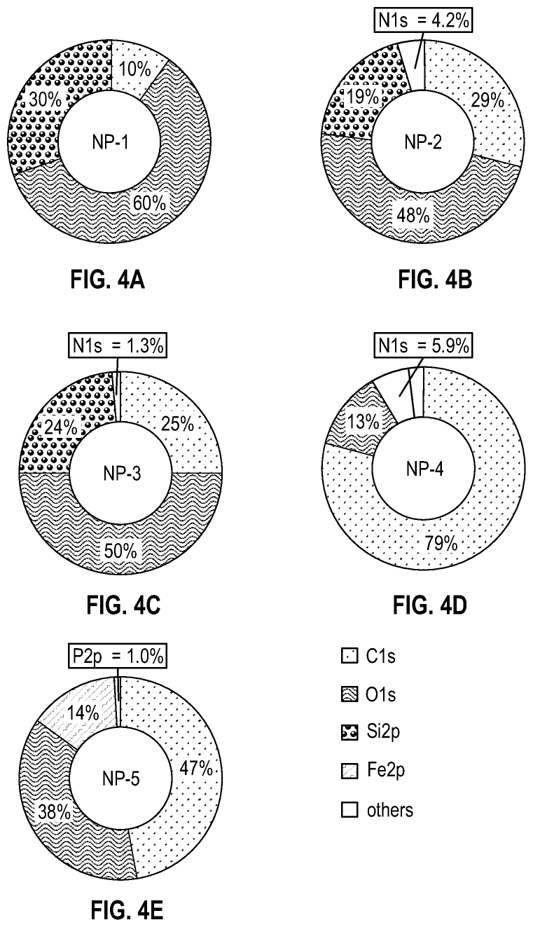

XPS performed on the NP powders showed installation of target functional groups to the surface of SPIONs (FIGS. 4A-4E). XPS spectra of NP-1, NP-2, and NP-3 showed no evidence of the Fe2p signal which further confirmed that these particles are thoroughly coated with silica. The presence of N1s and P2p signals in the spectra of silica- and GSP-functionalized SPIONs (NP-2, NP-3, and NP-5) confirmed the installation of target molecules to the surface of these particles. XPS of NP-4 also showed a prominent N1s signal along with a significant amount of carbon confirming the presence of polymethacrylamide at the surface, consistent with the TEM results shown in FIGS. 2C and 2D.

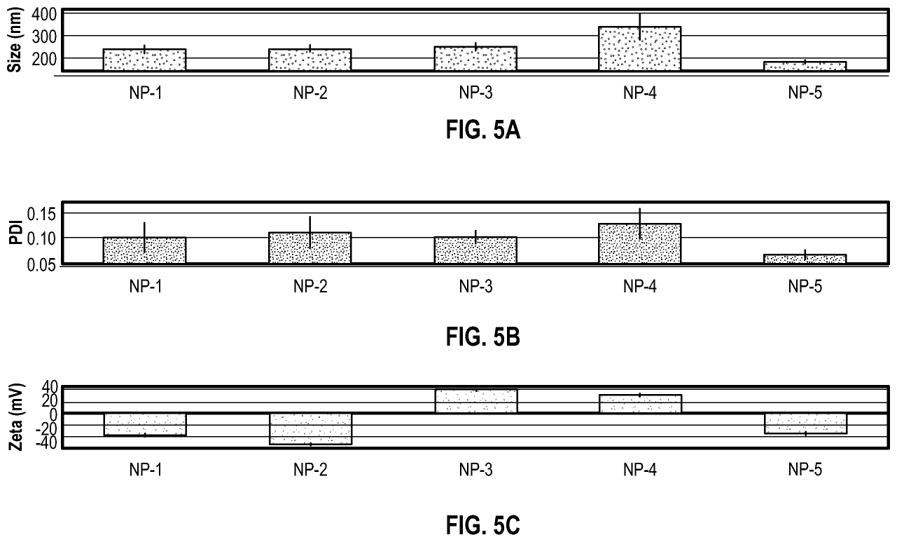

Additionally, DLS recorded on aqueous dispersion following synthesis showed that the NPs have hydrodynamic sizes in a range of approximately 200 nm to 350 nm, with polydispersity index (PDI) values varying from 0.05 to 0.15 (FIG. 5B). The zeta potential of NPs measured in 5% PBS reveals that NP-3 and NP-4 were positively charged (unlike NP-1), demonstrating that their surfaces were successfully modified with primary and tertiary amine functional groups, respectively (FIG. 4C). Additionally, a negative zeta potential value was measured for NP-2 and NP-5, which primarily resulted from the presence of surface carboxylic acid and phosphate functional groups, respectively. Overall, in-depth characterization including TEM, XPS, DLS, and zeta potential measurements confirmed the distinct characteristics of the NPs used herein.

### Tuning Nano-Bio Interactions

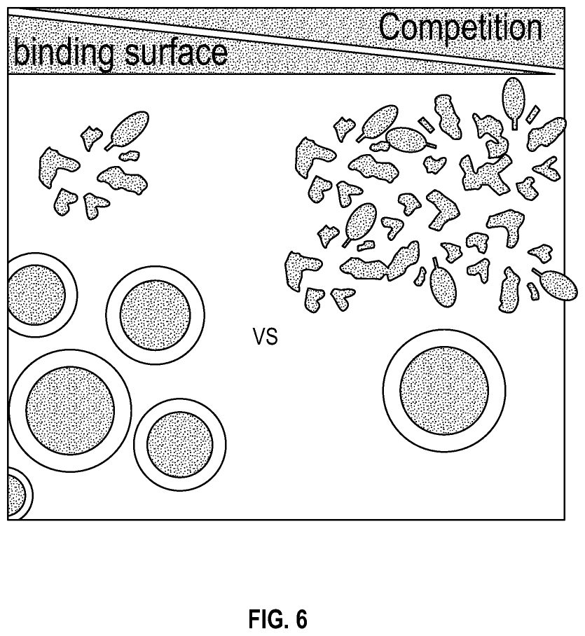

To investigate the relation among protein corona composition, physicochemical properties of NPs, and protein-binding competition, protein coronas formed at different NP-plasma ratios were quantitatively dissected (FIG. 6). For this experiment, a single pooled human plasma of deidentified healthy individuals was investigated. Protein-coronas were isolated and processed in a fully automated fashion using the Seer PROTEOGRAPH™. Peptides were identified and quantified with a trapped ion mobility LC-MS/MS pipeline (timsTOF-Pro), and raw data were processed using DIA-NN applying 1% FDR cutoff at the protein and peptide levels, yielding 2,081 proteins and 14,194 peptides (1,918 and 12,747 consistently identified in all replicates of at least one tested condition, respectively) across the five investigated NPs.

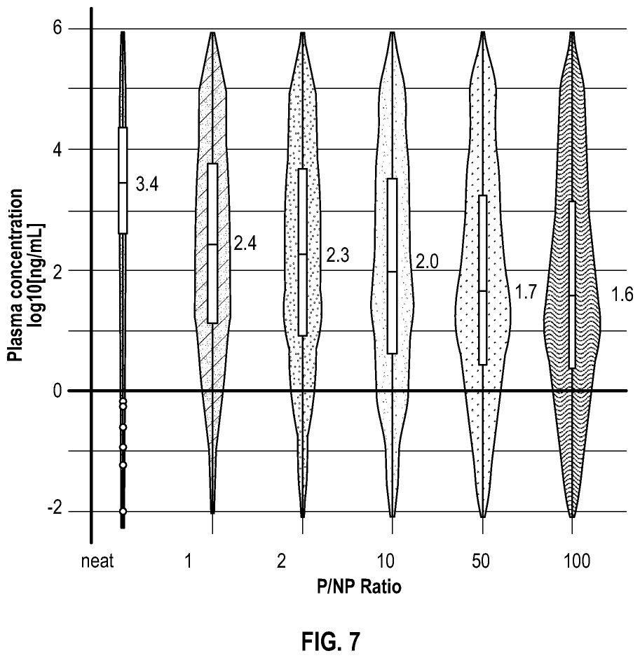

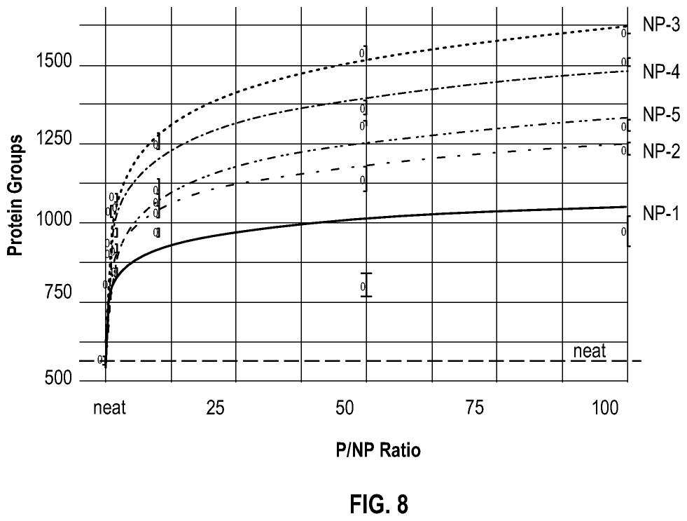

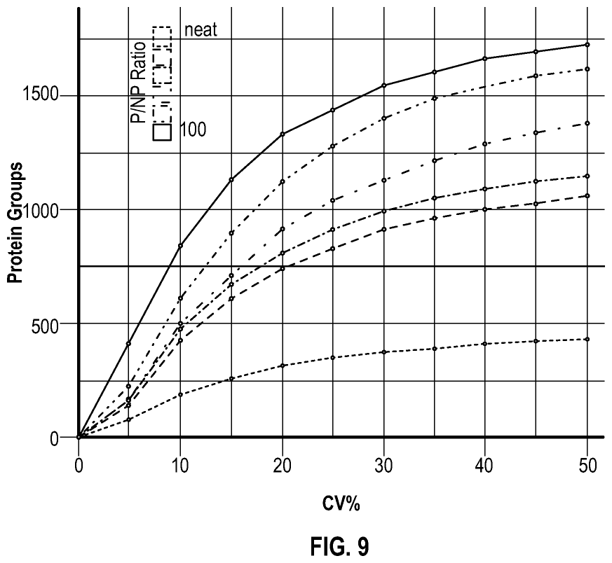

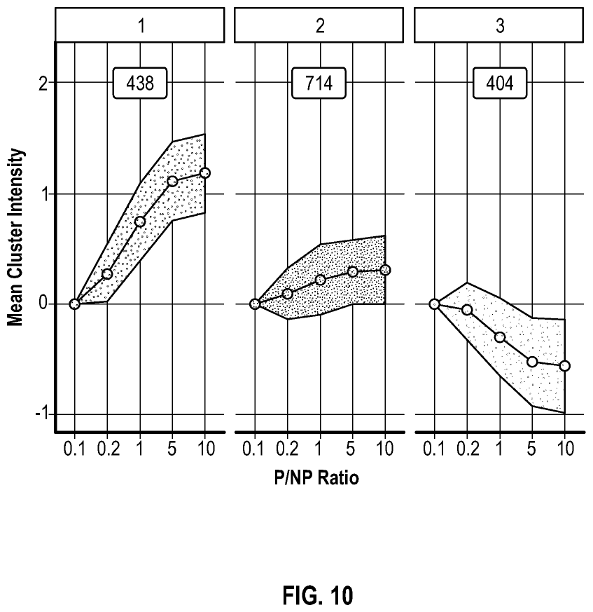

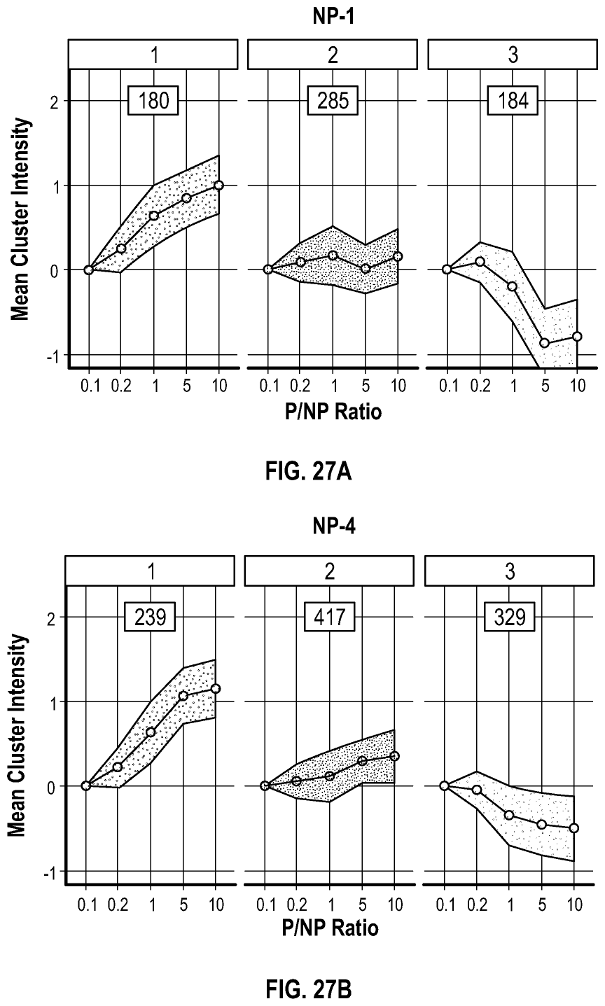

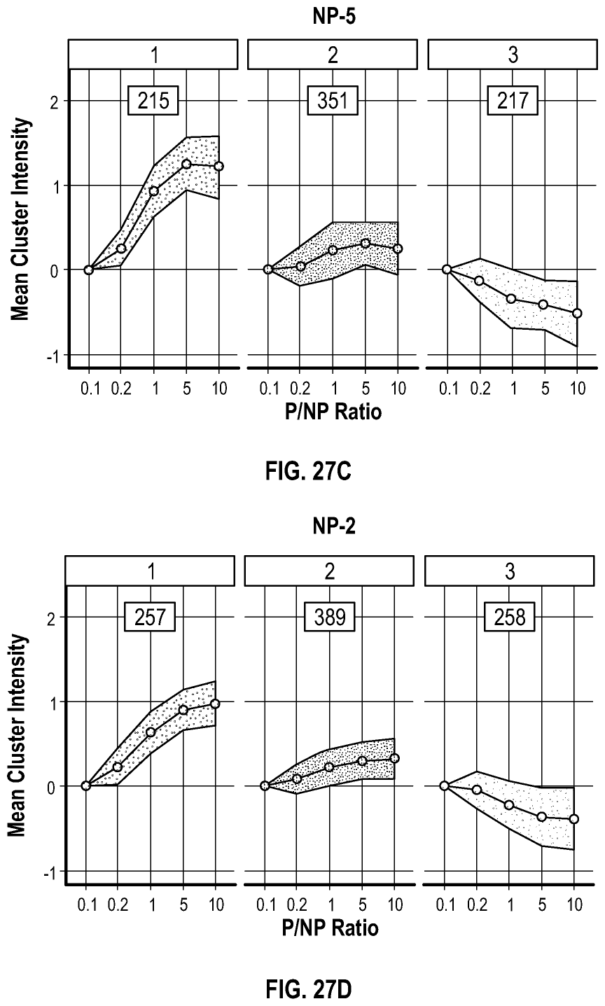

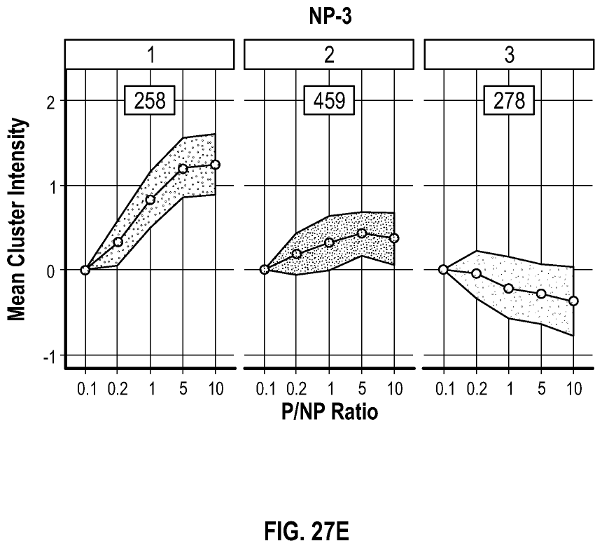

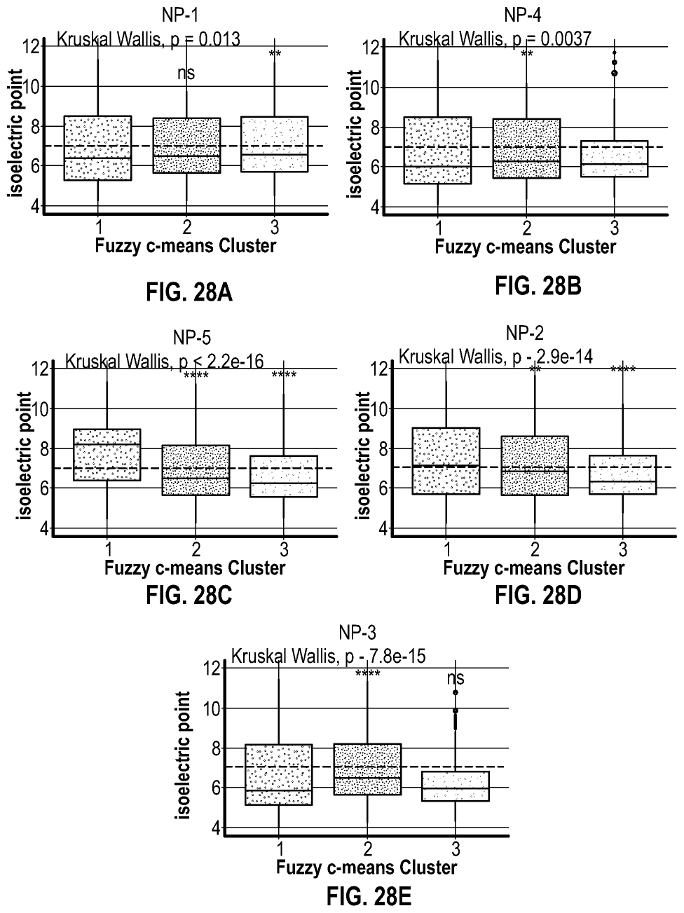

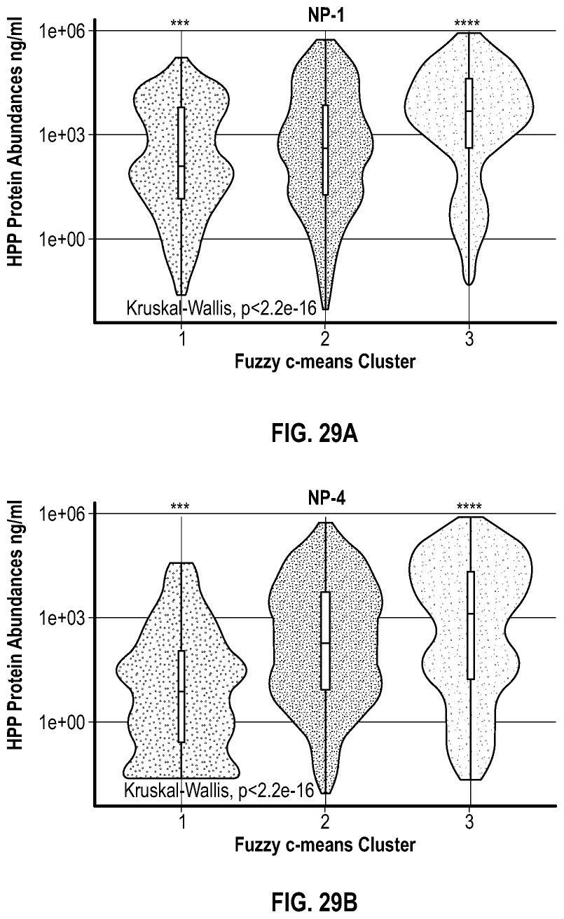

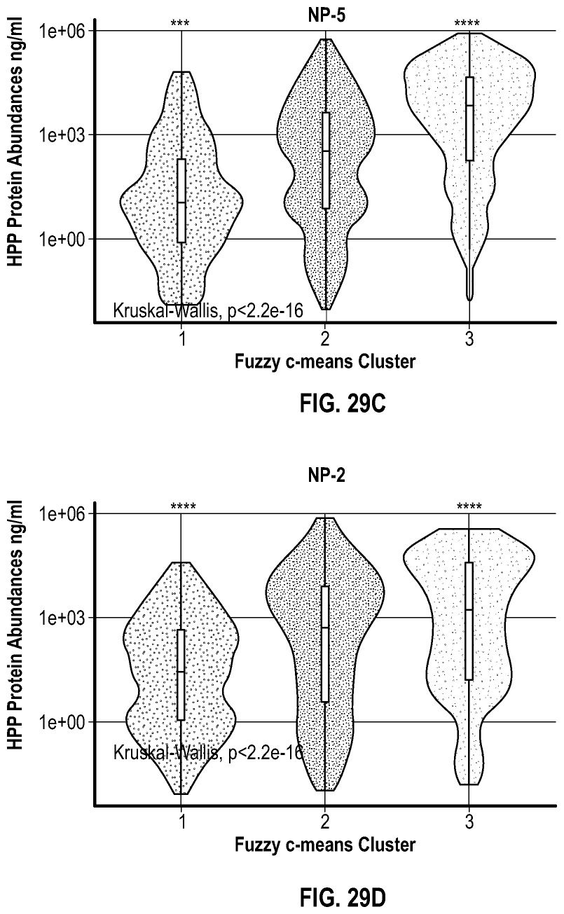

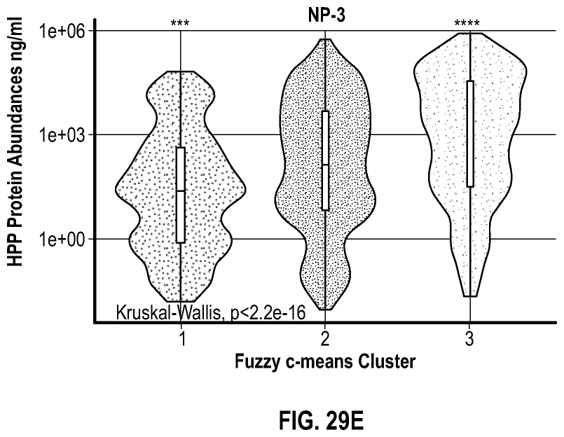

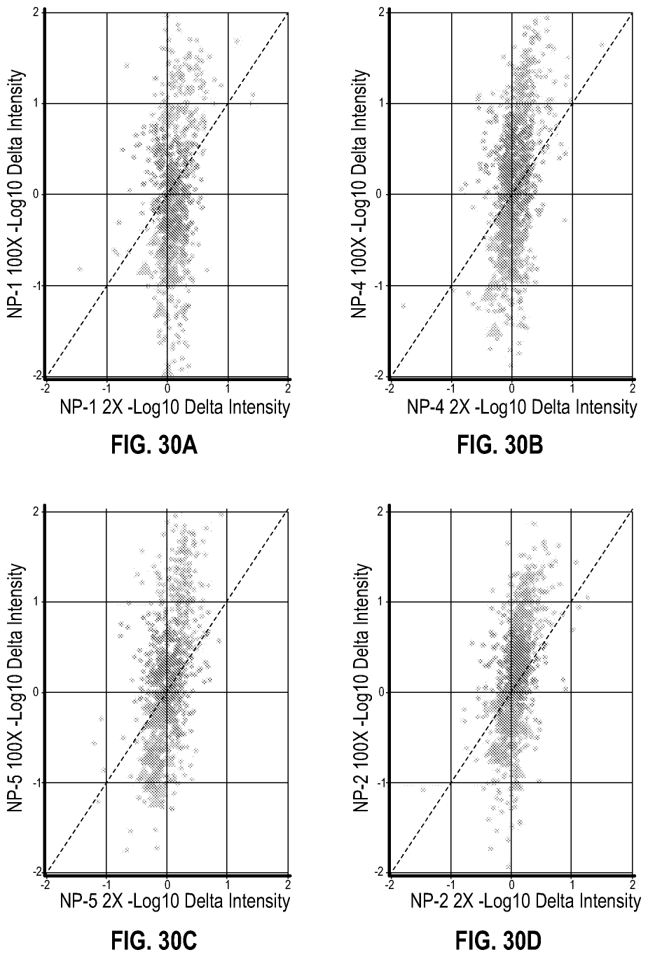

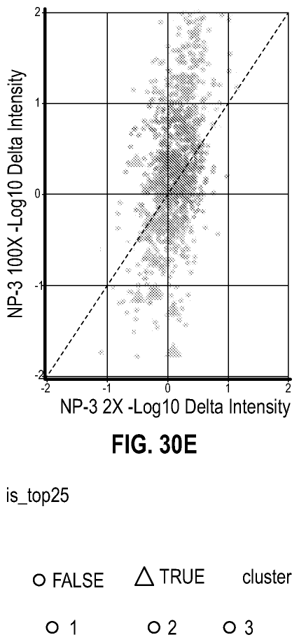

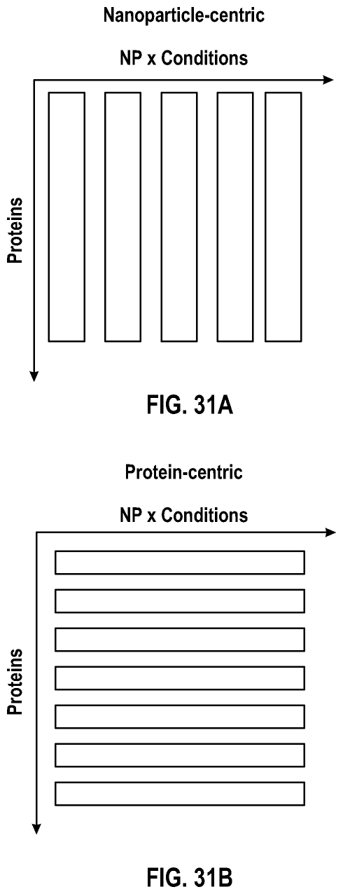

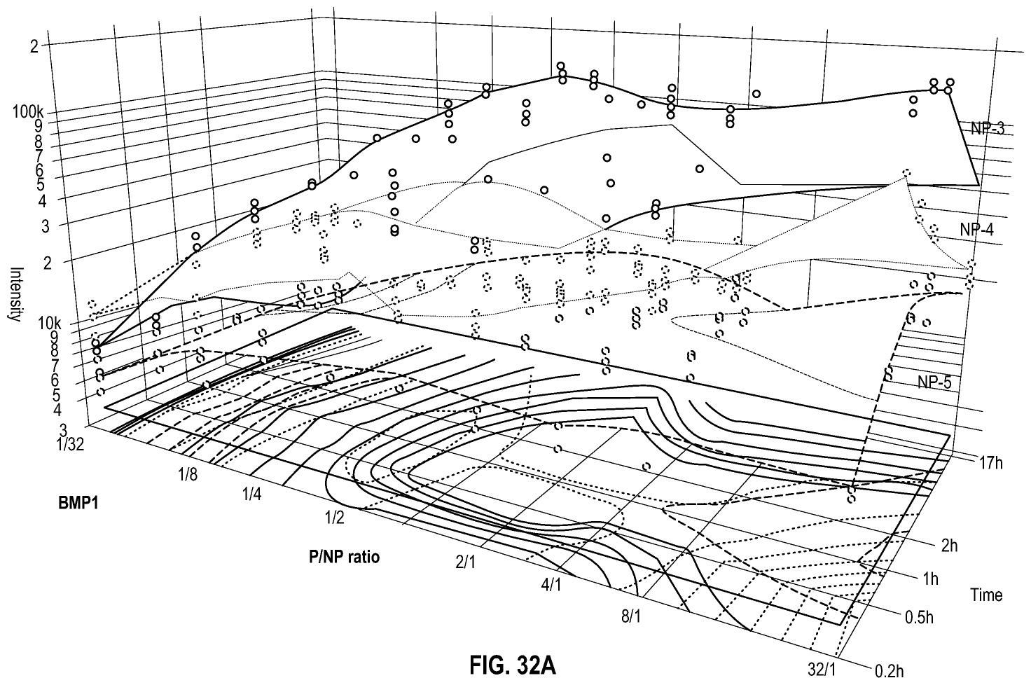

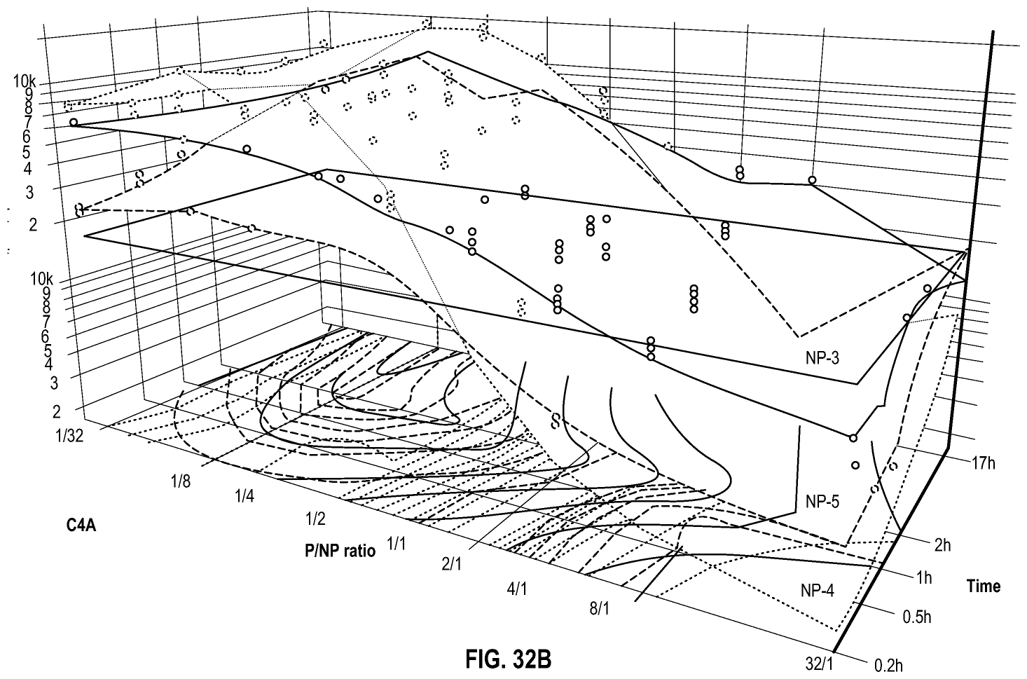

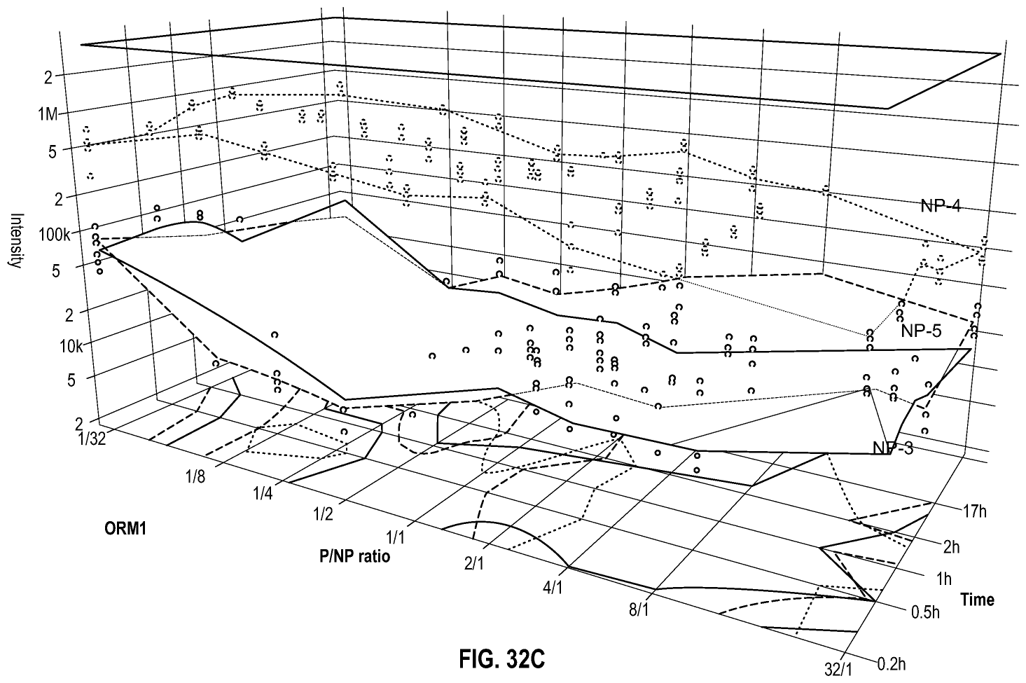

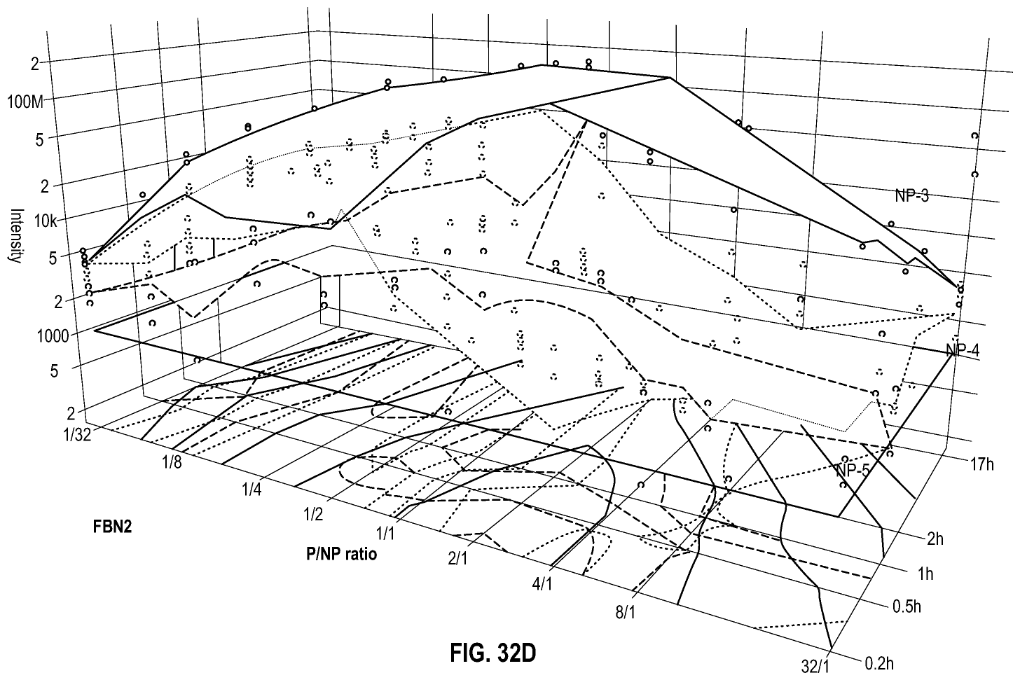

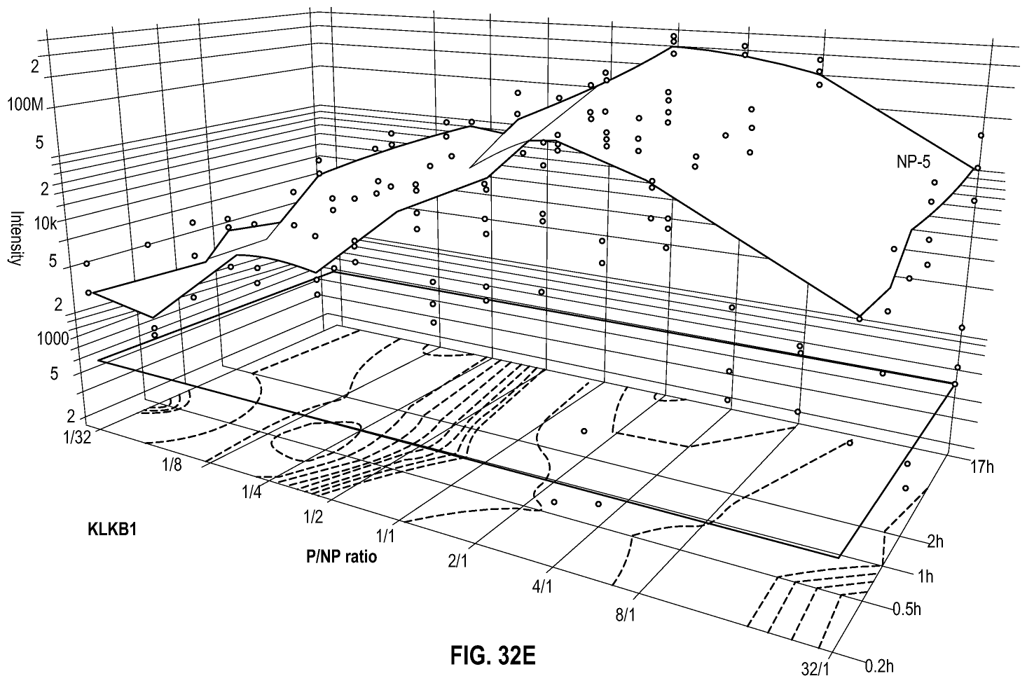

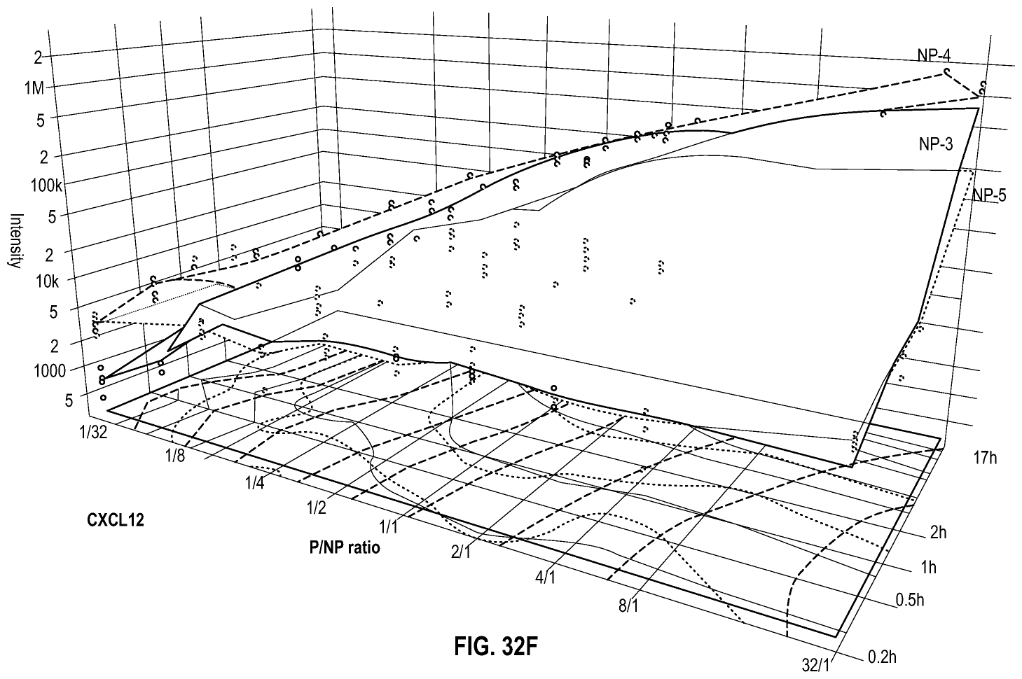

As effective binding surface area shrinks, lower abundance proteins were increasingly captured by individual NPs (FIG. 7), and the overall number of proteins identified and quantified (protein IDs) increased (FIG. 8). This improvement in individual NP performance compared to neat plasma ranged from 1.7× to 2.8× (NP-1 and NP-3, respectively), and comparing individual protein to nanoparticle-surface ratio (P/NP) ratios, it was found that between 1.2× (NP-1) and 1.7× (NP-3) improvements for protein identifications across a 1-100× P/NP ratio range. Then, the extent to which additional protein groups are quantified reproducibly was evaluated (FIG. 9). Higher P/NP ratios generally yielded more proteins at high precision, with hundreds of proteins being quantified with a coefficient of variation (CV)<10%. The changes to individual corona proteins by increasing the P/NP ratio for the panel of 5NPs (FIGS. 10-12) and for individual NPs (FIGS. 27A-27E, FIGS. 28A-28E, FIGS. 29A-29E, and FIGS. 30A-30E) were examined. It was found that hundreds of proteins steadily increased or decreased in their intensity (clusters 1 and 3 in FIG. 10 and FIGS. 27A-27E), indicating overall consistent remodeling of protein coronas as a function of the Vroman effect.

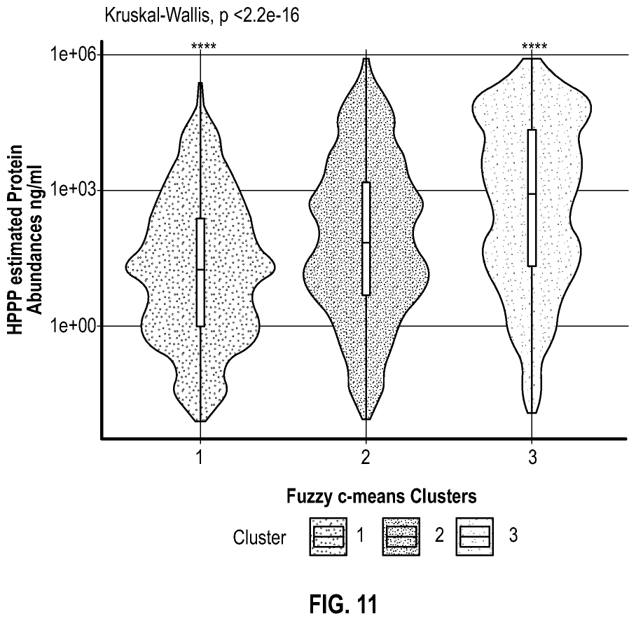

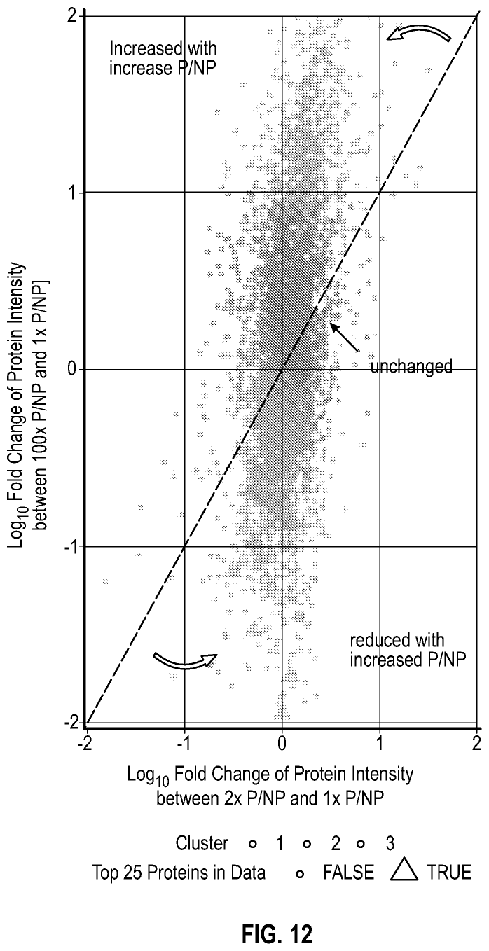

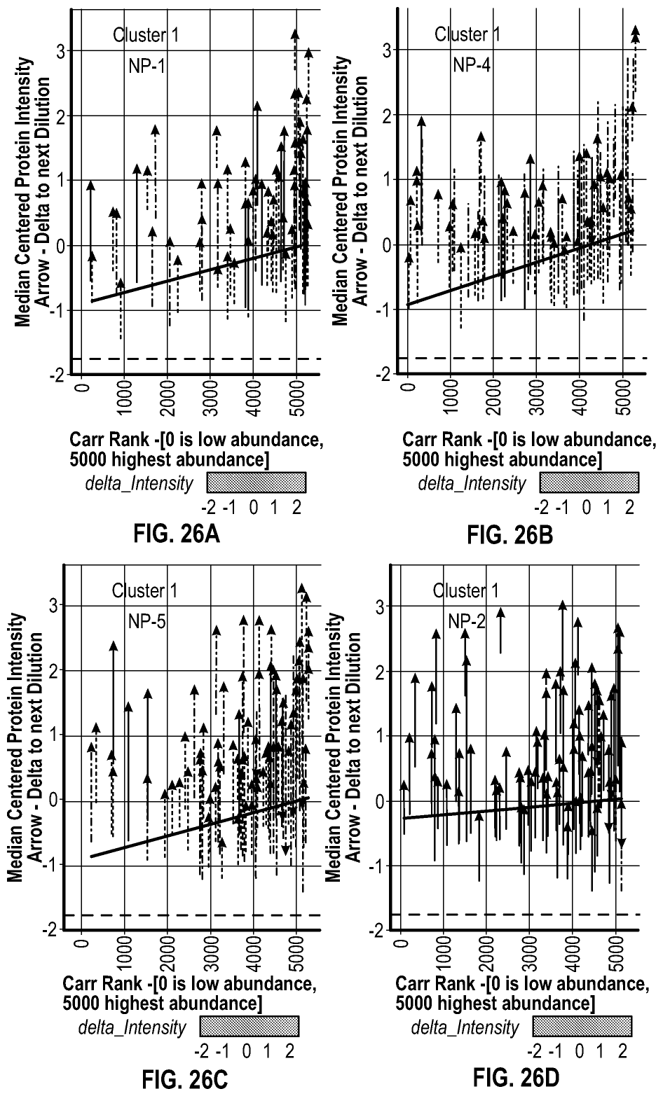

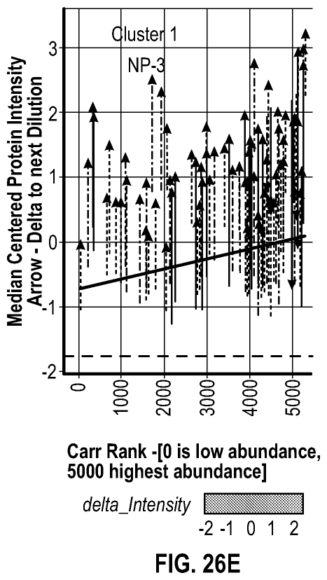

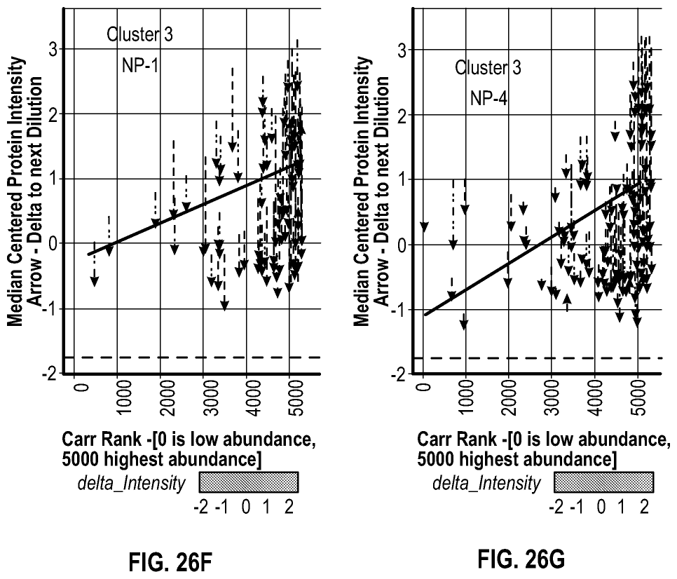

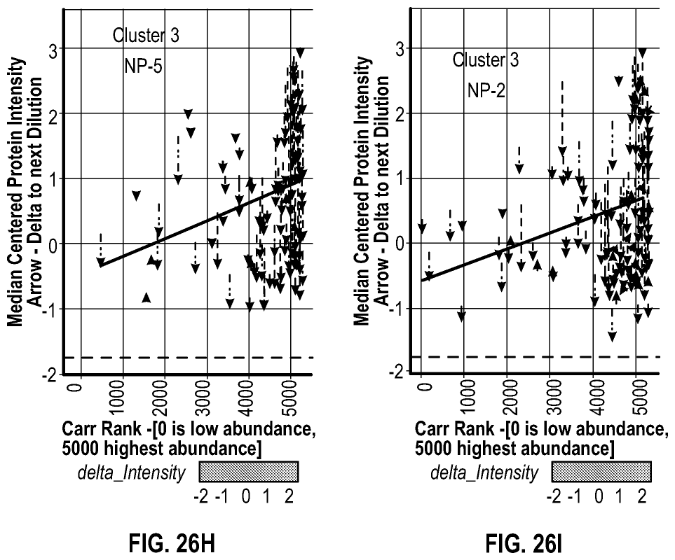

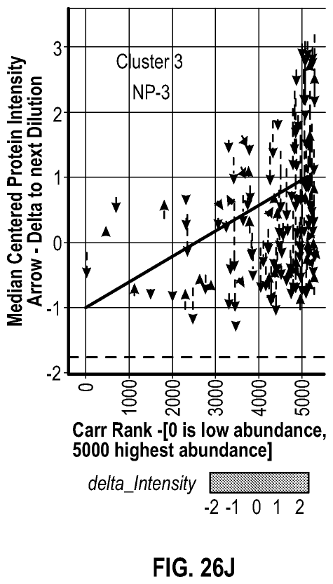

Consistent with an efficient dynamic range compression that boosts protein identification rates for downstream mass spectrometry detectors, signal reduction was stronger in the high-abundance range of the proteome (FIG. 11 and FIGS. 26A-26J). It was also evident when comparing a weak Vroman effect to a strong Vroman effect (FIG. 12). The divergent form the diagonal in FIG. 12 depicts the increasing dynamic range compression with a preferential enrichment of lower abundance proteins and depletion of high abundance proteins. In fact, the top 40 most abundant proteins according to the reference database were almost exclusively reduced in intensity (below diagonal line). Given that protein intensities were log-normally distributed, reducing the concentration of only a few high-abundance proteins dramatically reduced the signal redundancy and renders a large fraction of low-abundance proteins visible to the detector.

### Quantitative Dissection of Nano-Bio Interactions in Complex Bio Samples

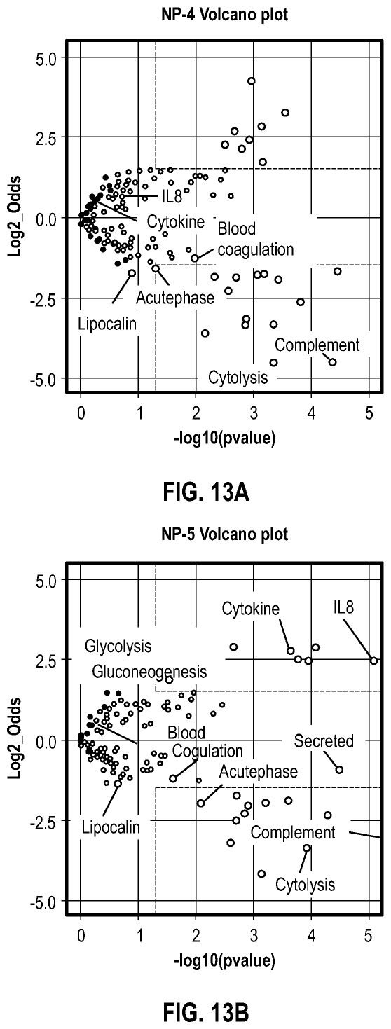

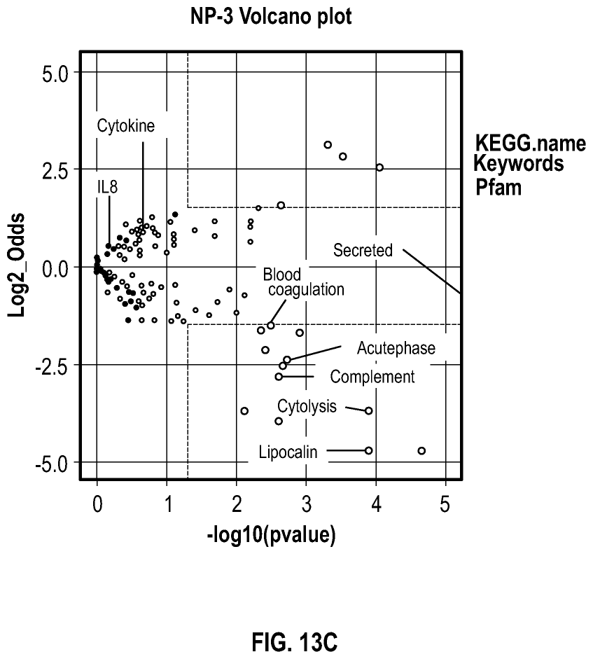

To investigate how a stronger Vroman effect translates to differential sequestration of functional classes of proteins, functional protein pathways that are enriched or depleted by tuning P/NP were determined (FIGS. 13A-13C). The NPs investigated showed common trends for depletion (varying effect) size of a some high-abundance protein families, such as ‘Acutephase’ and ‘Complement,’ while other the functional groups, such ‘cytokine’ and ‘IL8,’ show distinct enrichment patterns.

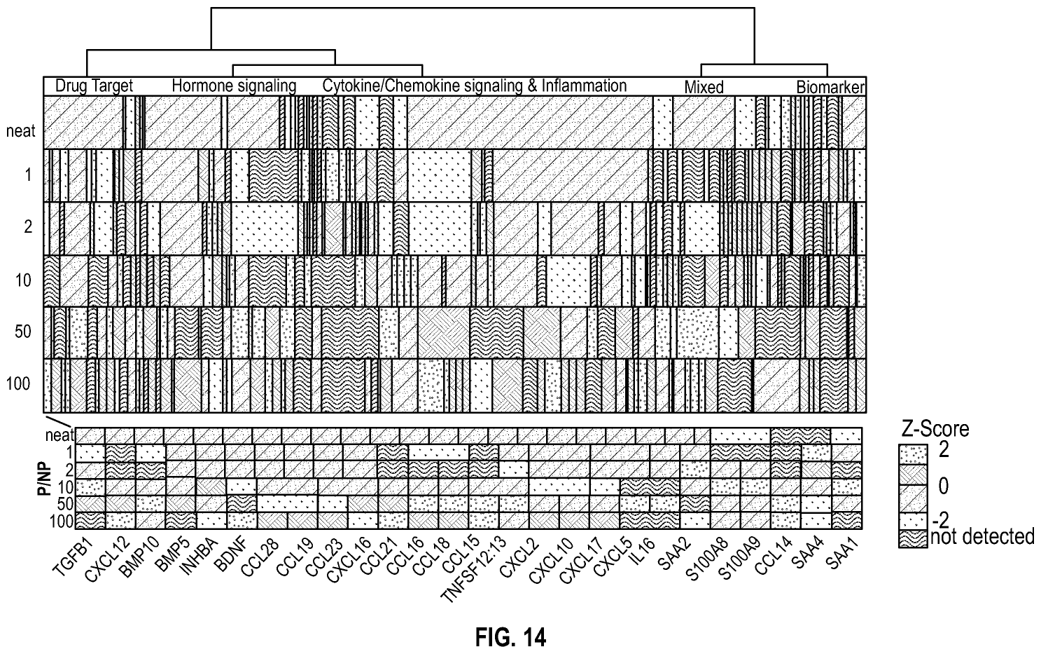

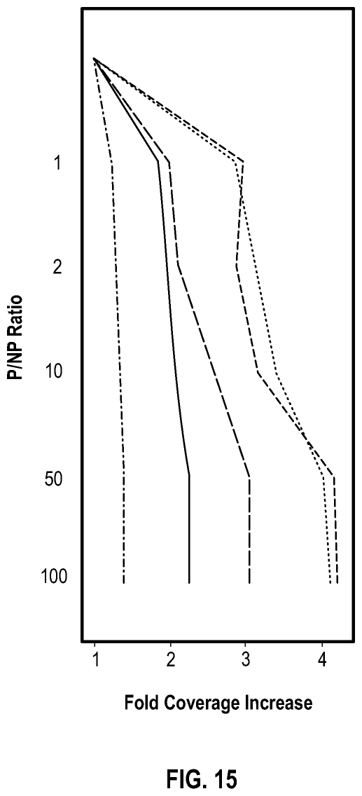

To determine whether the increased ID performance translates to better detection of clinically relevant proteins, the coverage of biological pathways as a function of P/NP was examined. FIG. 14 and FIG. 15 show the detection of protein biomarkers, protein drug targets, and proteins involved in hormone signaling, cytokine/chemokine signaling, and inflammation based on Uniprot Keywords, GOBP, GOMF, and KEGG terms. The heatmap confirmed improved coverage of cytokine/chemokine and hormone signaling proteins at the panel level (across all NPs) as the P/NP ratio increased. Compared to neat plasma, it was observed an improved coverage of each of these groups by 2- to 4-fold. Specifically, chemokines (e.g., CCL14-19, CXCL1, 2, 5), which are problematic to capture using a standard neat plasma workflow, were increasingly identified in the biosample at higher P/NP ratios. Chemokines are an important class of cytokines that can exert immune regulatory roles e.g., by guiding migration of immune cells (chemoattraction). Consistent identification of these low-abundance proteins is of high relevance to study immune homeostasis and for a variety of acute and chronic diseases.

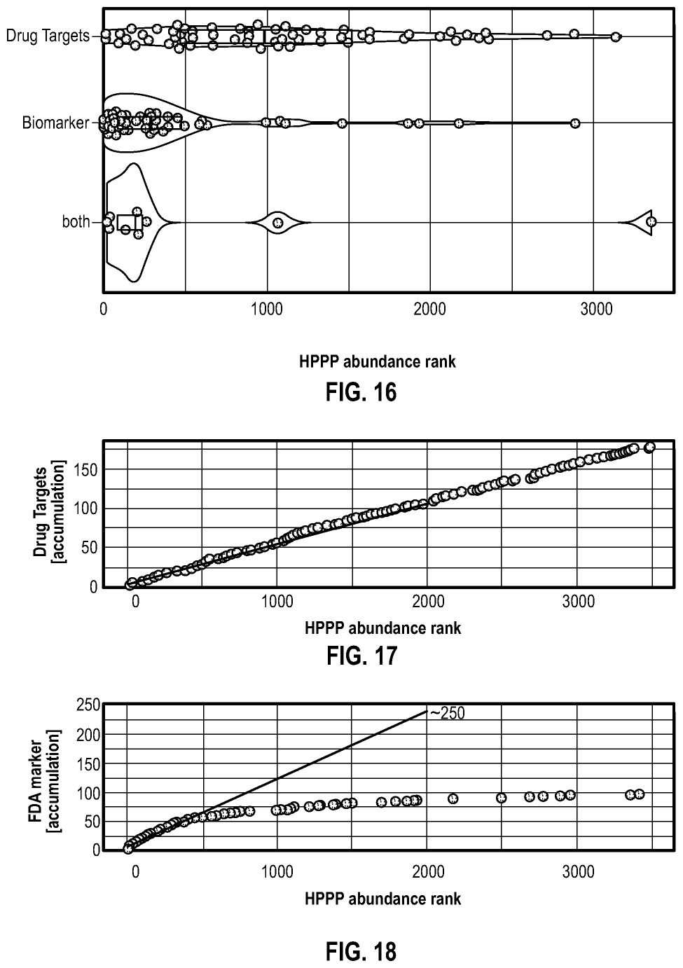

A moderate increase of proteins annotated as FDA approved biomarkers (1.5-fold) was found, which led to taking a closer look at this set of proteins as well as protein drug targets. Proteins that were quantified from these databases to the abundance rank estimation provided with the human plasma proteome project database, HPPP, were mapped. Compared to protein drug targets, protein biomarkers identified in the instant application were much more skewed to the high abundance range (FIG. 16). To estimate the utility of reproducible, large-scale access to the low abundance plasma proteome with NPs, the number of novel biomarkers by extrapolating from the biomarker frequency among the 500 most abundant proteins was estimated (FIG. 18). From this analysis, about 150 so far undetected protein biomarkers among the next 1500 proteins were estimated, which was more than double the number of biomarkers annotated in this dataset to date. In contrast, proteins that are known drug targets and are often studied in cell lines with thousands instead of hundreds of proteins being accessible with conventional proteomics workflows, are more evenly distributed across the abundance range of HPPP (FIG. 17).

Overall, the data demonstrates that tuning the Vroman effect and competitive binding can elicit significant and NP-specific protein corona remodeling, resulting in individual protein and protein functional group and annotation changes. These diversified protein coronas can not only improve ID performance in proteomics assays, but can efficiently capture a larger proportion of the druggable plasma proteome in addition to accessing an untapped pool of understudied proteins with biomarker potential.

### Modeling NP-Corona Kinetics and Neat Plasma Inference

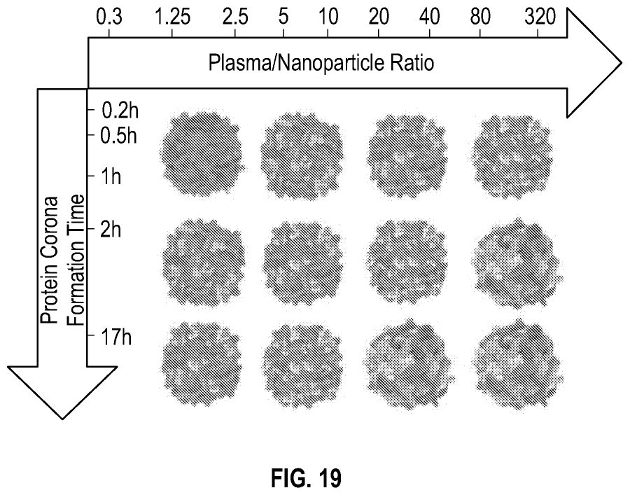

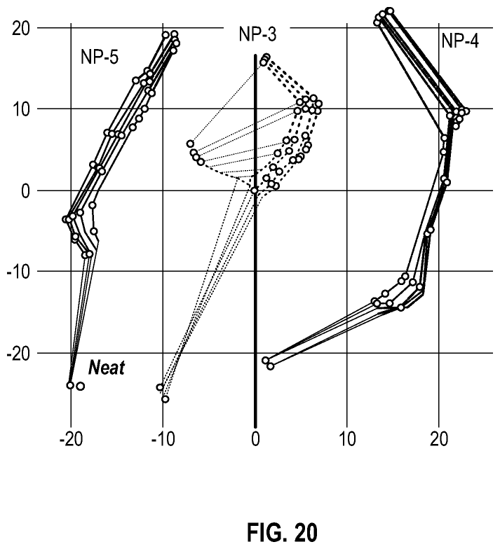

To understand the dynamics of corona formation, NP corona profiling for NP-5, NP-3, and NP-4 particles were expanded, and the protein corona formation time course data (10 min, 1 h, 2 h, 17 h) for each P/NP ratio, measuring the intensities of 3184 individual protein groups across 534 samples, was added (FIG. 19). Then, the UMAP method was used to overview the differences and similarities between the intensity profiles of corona proteins at each condition (FIG. 20). It could be immediately observed that the replicates cluster tightly by condition, confirming the reproducibility of the Proteograph™ workflow. It was noted that, along with the type of NP used, the P/NP ratio is the second major factor of the variance in corona composition. The profiles for lower P/NP ratios (less competition, weaker Vroman effect), as expected, located closer to the neat plasma. In comparison, time (with exception of the 17 h timepoint for NP-3) had minor impact on corona profiles, suggesting that some corona proteins approach near-equilibrium state within minutes.

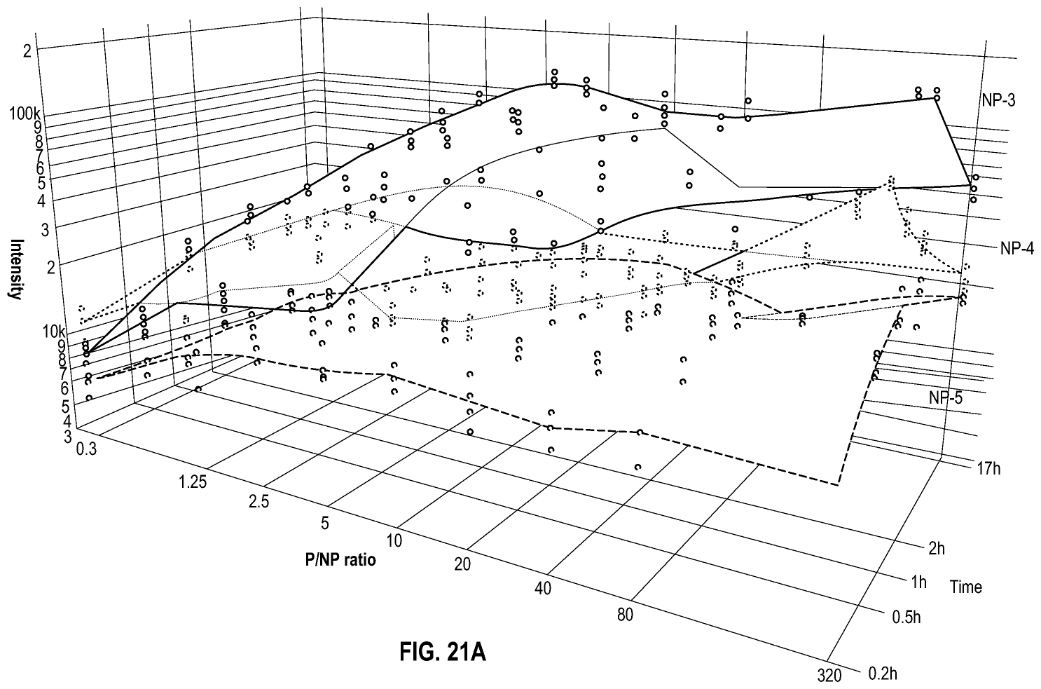

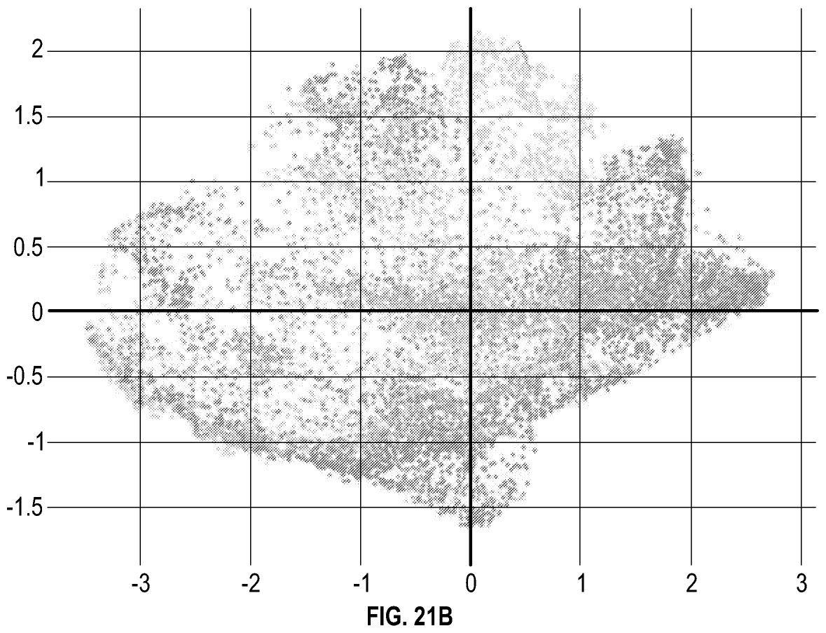

These rich data also allowed looking at the intensity profile of a specific protein across all NPs, P/NP ratios and timepoints (FIG. 21A). By employing UMAP once again, this time to compare the intensity profiles of individual proteins (FIG. 21B), a complex landscape, indicating a wide diversity of NP-protein interaction modes across plasma proteome, was observed. Importantly, the clouds of intensity profiles obtained by different NPs largely overlapped, suggesting that, while NPs have unique binding specificities, the dynamics of their interaction with proteins can be governed by the same mechanisms.

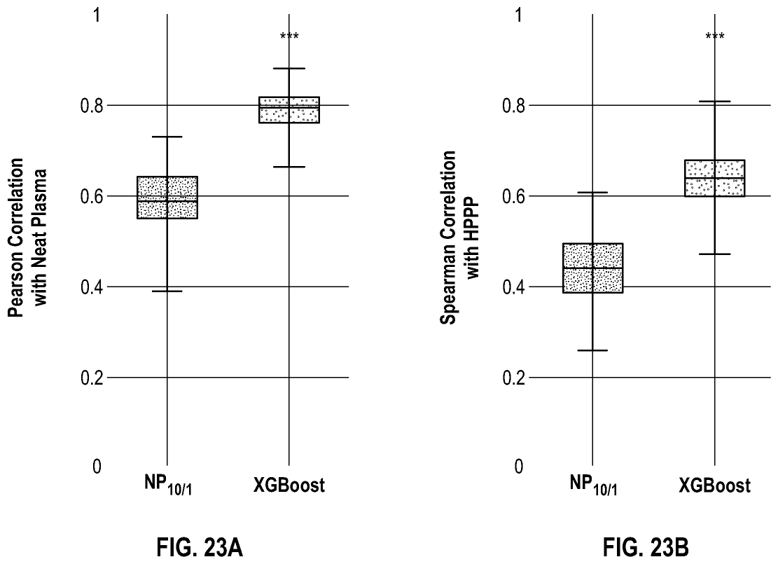

The XGBoost software library was used to train a machine learning algorithm to predict protein concentration in the original plasma from its intensity profile across NP conditions. The algorithm was trained using input protein measurements across all NPs, P/NP ratios and timepoints, and reference data measured from neat plasma or reported in the HPPP database. In comparison to the protein intensity measured in NP coronae at 1 h, P/NP=10/1, the predictor improved the correlation with the measured neat intensities from 58% to 79% and from 42% to 64% for protein concentrations reported in HPPP (FIGS. 22A, 22B, 23A, and 23B). A significant improvement in accuracy was observed by about 25%. The machine learning model demonstrated decompressing the biological information contained in protein coronas and reconstructing the original biosample composition, to extent that the original biosample composition was measured with a reference neat measurement.

Overall, the reconstructed neat protein intensities fell below the detection threshold of the mass spectrometer, which demonstrate the ability to infer neat plasma compositions beyond the dynamic range capacity of conventional scalable plasma proteomics workflow (FIG. 24). These results demonstrate the feasibility of combining ML with quantitative deep NP workflows to protein coronas and reconstruct the low-abundance fraction of human plasma proteome quantitatively and accurately.

### Nanoparticle (NP) Characterization

Dynamic light scattering (DLS) and zeta potential measurements were performed using a Zetasizer Nano ZS from Malvern Instruments. Solutions of NPs (as synthesized) were diluted to a concentration of 5 mg/mL (using 18 m (2 water) and sonicated for 10 min prior to testing. Samples were then diluted to approximately 0.02 wt % in DI water and % 5 PBS (pH=7.4) for DLS and zeta potential tests, respectively. DLS was performed at 25° C. in disposable polystyrene semi-micro cuvettes (from VWR). Zeta potential was also measured using disposable folded capillary cells from Malvern at 25° C. following 1 min temperature equilibration time. All reported DLS, PDI, and zeta values are an average of 3 automatic runs performed on each NP sample.

Low- and high-resolution NP imaging was performed using a FEI Tecnai transmission electron microscope (TEM) with an accelerating voltage of 200 kV. The TEM grids were prepared by drop-casting 2 μL of NP dispersions (0.1 mg/mL) in water-methanol mixture (25/75 v/v %) on lacey holey grids from Ted Pella, followed by about 24 h of sample drying in a vacuum desiccator. The shell thicknesses of NPs were measured by image J software through analyzing over 50 individual particles from multiple regions of the TEM grid.

XPS was performed by using a ThermoScientific ESCALAB 250e III on the NP fine powders kept sealed and stored under desiccation. All measurements were performed following uniform deposition of NP samples on the carbon tape from Ted pella Inc. A monochromatic A1 K-alpha X-ray source (50 W and 15 kV) was used over a 500 μm² scan area with a pass energy of 150 eV, and all binding energies were referenced to the C-C peak at 284.8 eV. The atomic concentration of elements at the surface was determined by averaging the results from 3× survey scans performed on three different locations of each sample.

### Proteograph™ Assay

Protein corona preparation and proteomic analysis. In the dilution series experiment, NPs were reconstituted in deionized water to the appropriate concentration for each P/NP ratio ranging from 1× to 100×. In the time series experiment, which was done using NP3-5, the P/NP ranged from 0.3× to 32×, and the corona formation incubation time ranged from 10 min to overnight (17 h). At P/NP=1, the concentrations for NP1-5 were 60, 170, 60, 40, and 40 μg/μL, respectively. The same volume of plasma and NP were mixed together to form the protein corona, resulting in 30, 85, 30, 20, and 20 μg/μL for NP1-5 at 1×, respectively. The corona formation, wash, protein lysis and alkylation, digestion, and peptide cleanup were done on Proteograph as described previously (Blume et al., 2020a). After peptide elution, peptide concentration was measured by a quantitative fluorometric peptide assay kit from Thermo Fisher Scientific (Waltham, MA). The peptides were then dried using a Speed Vac. Finally, the dried peptides were reconstituted in the provided reconstitution buffer to a concentration of 0.125 μg/μL.

### Data-Independent Acquisition LC-MS/MS

For Data-Independent Acquisition (diaPASEF), 500 ng of peptides in 4 μL of reconstitution buffer was used (constant mass MS injection). Each sample was subjected to an UltiMate3000 nanoLC system coupled with a Bruker timsTOF Pro mass spectrometer using a trap-and-elute. First, the peptides were loaded on a Acclaim™ PepMap™ 100 C18 (0.3 mm ID×5 mm) trap column and then separated on a 50 cm uPACIM analytical column (PharmaFluidics, Belgium) at a flow rate of 1 μL/min using a gradient of 5-25% solvent B (0.1% FA, 100% ACN) mixed into solvent A (0.1% FA, 100% water) over 22 min, resulting in a 33 min total run time. The mass spectrometer was operated in diaPASEF mode using ion mobility range of 0.57-1.47 cm²/V·s with 100 ms accumulation time.

### DIA Raw Data Processing

DIA data was processed using the DIA-NN analytical software (version 1.8) in library-free mode with the “-relaxed-prot-inf” tag enabled. Mass accuracy and MSI accuracy were set to 10 ppm as it was recommended for diaPASEF datasets. The rest of the parameters were set to default. The FDR cutoff at both precursor and protein level (Lib.Q. Value and Lib.PG.Q. Value) was set to 0.01. Two replicates of the 100 P/NP ratio for NP-3 have been removed from any downstream analysis; one of them was excluded due to issues with the total ion chromatogram and the other one was an outlier in terms of peptide yield compared to the average yield for this condition (7 μg vs. 3 μg).

### Data Analysis and Visualization

Data was analyzed in R (v4.0.5) and visualized with ggplot2.

### (1) Clustering

Ratios between the lowest and subsequent dilution median centered protein intensities were computed to generated protein intensities relative to the 1× dilution. These relative intensities were plotted across the dilution series. Ppclust is used to partition data using the Fuzzy C-means clustering. Proteins are partitioned based on bootstrapped I=300 clustering results with n=3 centers. The proteins were assigned to clusters based on maximal average membership probability for each NP across the dilution series. A minimal cluster probability of 0.5 was used to filter proteins that are not strongly partitioned into any cluster. A clustered median intensity for all proteins in the cluster is shown as a trace with confidence bands showing the mean standard error are computed using R. Median proteins intensity traces within each cluster are plotted for each particle.

### (2) Enrichment Analysis

Generalized hypergeometric tests for enrichment of Uniprot, Keywords, gene ontology (GOMF) molecular function terms, database of protein families Pfam, and Kyoto encyclopedia of genes, genomes (KEGG) pathways were used for proteins represented in the increasing, constant and decreasing clusters filtered for a Benjamini Hochberg FDR of 5%.

### (3) UMAP Embedding of the NP Concentration and Corona Formation Time Variation Dataset

The analysis was performed in Julia (v1.7.2). The protein group intensities data were taken from the pg_matrix.tsv DIANN output table. The missing intensity values were imputed using the same method as in Perseus. The UMAP embedding was done with the uwot R package (v0.1.11). The MS samples UMAP 2D embedding was done using the log₂-transformed protein group intensities with the default UMAP method parameters, except spread 3 and min_dist 0.2. For the UMAP 2D embedding of protein group intensity profiles for individual NPs, the log₂-transformed intensities of each protein group-NP pair were adjusted by subtracting the log₂-transformed intensity of that protein group at the reference condition (P/NP ratio of 10/1, 1 h NP incubation time) to correct for the differences in abundance across protein groups. The parameters of uwot were standard, except n_neighbors=50.

(4) Predicting Neat Protein Abundance from NP Concentration and Corona Formation Time Variation Data

For training the neat intensity prediction model, the same log₂-transformed protein group intensities adjusted by the reference condition as for the UMAP embedding were used. 75% of the protein group-NP pairs were randomly assigned to the training data, the remaining 25% were assigned to the testing data.

For training the model XGBoost (v 1.5.2) via the XGBoost.jl Julia package (v1.5.2) was used. For protein groups with quantified neat plasma intensity, the classic squared error loss function was used. To handle the cases of protein groups with missed intensities in the neat plasma, the used custom loss function was

$$loss = Logistic ( a {log}_{2} Intensity + b ) ,$$

which corresponds to the probability of detecting the MS signal with expected Intensity. The a and b parameters of the sigmoid curve were estimated from the technical replicates data of the same MS instrument. XGBoost was run with the default parameters, except gamma=2 and max_depth=2.

For estimating the performance of the prediction method, 100 independent samples from the test data with measured neat intensities were subjected, drawing 100 random protein group-NP pairs (without replacement) at each iteration. For each protein group, the Pearson correlation between its measured neat intensity and its intensity at the reference NP condition or between the neat intensity and the intensity predicted by the trained XGBoost model was calculated. The significance was estimated by the Mann-Whitney test. The same procedure was applied to estimate the significance of the Spearman rank correlation improvement between the intensities reported in the HPPP database (v2021) and the reference NP condition intensities or predicted intensities, respectively.

### Databases

MarkerDB is an online database of molecular biomarkers (chemical, protein, DNA and karyotypic). The instant application contained analysis to the protein biomarkers, where 87% are clinically approved, and the other 13% are classified as investigative, research-use only, or pre-clinical. MarkerDB extracted all protein biomarker data from primary literature. From the 142 protein biomarkers in MarkerDB, the 105 proteins that matched to the HPPP via UniprotID were used.

Pharos (https://pharos.nih.gov/) is a web interface used to browse The Target Central Resource Database (TCRD) (http://juniper.health.unm.edu/tcrd/). The TCRD combines data from a variety of over 50 sources including target expression data from GTex, the Human Protein Atlas, UniProt, and TISSUES. The proteins within this database are classified based on the target development levels (TDL), Tclin, Tchem, Tbio and Tdark. In the instant application, only those protein targets labeled Tclin, which are known protein targets for approved drugs and is in total 659 human proteins, were used. Of these 659, 27 were target proteins in plasma as determined by the target expression data, 80 matched to our dataset) and 188 matched to the HPPP.

The FDA-approved protein biomarkers were matched to the HPPP for the concentration found in plasma.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A computer-implemented method for training a machine learning algorithm for molecule quantification comprising: providing an input dataset comprising one or more features that represent changes in quantities for a plurality of molecules with respect to one or more physicochemical parameters, wherein the changes are measured using at least a first condition; processing, using the machine learning algorithm, the input dataset to generate an output value; and adjusting one or more numerical parameters of the machine learning algorithm based on a loss function based at least in part on the output value, such that the output value accounts for a difference between (i) the quantities for at least a portion of the plurality of molecules and (ii) reference quantities for a plurality of reference molecules, wherein the reference quantities are measured using at least a second condition.

Embodiment 2. The computer-implemented method of Embodiment 1, wherein the first condition comprises binding the plurality of molecules to a surface.

Embodiment 3. The computer-implemented method of Embodiment 2, wherein the surface comprises a sensor element surface.

Embodiment 4. The computer-implemented method of Embodiment 3, wherein the sensor element surface comprises a particle surface.

Embodiment 5. The computer-implemented method of Embodiment 4, wherein the particle surface is a nanoparticle surface.

Embodiment 6. The computer-implemented method of Embodiment 4, wherein the particle surface is a microparticle surface.

Embodiment 7. The computer-implemented method of any one of Embodiments 4-6, wherein the particle surface comprises pores.

Embodiment 8. The computer-implemented method of any one of Embodiments 2-7, wherein the binding is via adsorption.

Embodiment 9. The computer-implemented method of any one of Embodiments 2-8, wherein the binding is non-specific.

Embodiment 10. The computer-implemented method of any one of Embodiments 2-9, wherein the binding is specific.

Embodiment 11. The computer-implemented method of any one of Embodiments 4-10, wherein the plurality of molecules forms a corona on the particle surface.

Embodiment 12. The computer-implemented method of any one of Embodiments 1-11, wherein the quantities comprise measured intensities.

Embodiment 13. The computer-implemented method of Embodiment 12, wherein the measured intensities comprise mass spectrometry (MS) intensities.

Embodiment 14. The computer-implemented method of Embodiment 13, wherein the MS intensities comprise peptide intensities, protein group intensities, peptide group intensities, or combinations thereof.

Embodiment 15. The method of any one of Embodiments 1-14, wherein the plurality of molecules comprises a plurality of proteins.

Embodiment 16. The method of Embodiment 15, wherein the input dataset comprises measured intensities of a plurality of peptides, wherein the plurality of peptides is derived from the plurality of proteins.

Embodiment 17. The computer-implemented method of any one of Embodiments 13-16, wherein the MS intensities comprise small molecule intensities.

Embodiment 18. The computer-implemented method of any one of Embodiments 13-17, wherein the MS intensities are based on data-independent acquisition (DIA) MS, data-dependent acquisition (DDA) MS, or both.

Embodiment 19. The computer-implemented method of any one of Embodiments 13-18, wherein the MS intensities are based on liquid-chromatography tandem mass spectrometry (LC-MS/MS).

Embodiment 20. The computer-implemented method of any one of Embodiments 12-19, wherein the measured intensities comprise the fluorescence signals.

Embodiment 21. The computer-implemented method of any one of Embodiments 12-20, wherein the measured intensities comprise an induced current.

Embodiment 22. The computer-implemented method of any one of Embodiments 12-21, wherein the measured intensities are obtained using a nanopore sensor.

Embodiment 23. The computer-implemented method of any one of Embodiments 12-22, wherein the measured intensities are obtained using an immunoassay.

Embodiment 24. The computer-implemented method of any one of Embodiments 12-23, wherein the quantities are determined using a nucleic acid sequencer.

Embodiment 25. The computer-implemented method of any one of Embodiments 1-24, wherein the reference quantities comprise measured intensities.

Embodiment 26. The computer-implemented method of Embodiment 25, wherein the measured intensities comprise mass spectrometry (MS) intensities.

Embodiment 27. The computer-implemented method of Embodiment 26, wherein the MS intensities comprise peptide intensities, protein group intensities, or both.

Embodiment 28. The computer-implemented method of Embodiment 26 or 27, wherein the MS intensities comprise small molecule intensities.

Embodiment 29. The computer-implemented method of any one of Embodiments 26-28, wherein the MS intensities are based on data-independent acquisition (DIA) MS, data-dependent acquisition (DDA) MS, or both.

Embodiment 30. The computer-implemented method of any one of Embodiments 26-28, wherein the MS intensities are based on liquid-chromatography tandem mass spectrometry (LC-MS/MS).

Embodiment 31. The computer-implemented method of any one of Embodiments 25-30, wherein the measured intensities comprise the fluorescence signals.

Embodiment 32. The computer-implemented method of any one of Embodiments 25-31, wherein the measured intensities comprise an induced current.

Embodiment 33. The computer-implemented method of any one of Embodiments 25-32, wherein the measured intensities are obtained using a nanopore sensor.

Embodiment 34. The computer-implemented method of any one of Embodiments 25-33, wherein the measured intensities are obtained using an immunoassay.

Embodiment 35. The computer-implemented method of any one of Embodiments 25-34, wherein the quantities are determined using a nucleic acid sequencer.

Embodiment 36. The computer-implemented method of any one of Embodiments 1-35, wherein the one or more physicochemical parameters comprise: sample to surface ratio, incubation time, pH, salt concentration, ionic strength, solvent composition, solvent dielectric constant, crowding agent concentration, temperature, sample composition, surfactant concentration, concentration of enzymes, activity of enzymes, chemical reactions, concentrations of small molecules, surface chemistry, or any combination thereof.

Embodiment 37. The computer-implemented method of Embodiment 36, the one or more physicochemical parameters comprise the sample to surface ratio.

Embodiment 38. The computer-implemented method of Embodiment 37, wherein the sample to surface ratio comprises (i) volume of sample to surface area of the surface, (ii) volume of sample to mass of a substrate comprising the surface, (iii) mass of sample to surface area of the surface, or (iv) mass of sample to mass of the substrate comprising the surface.

Embodiment 39. The computer-implemented method of Embodiment 38, wherein the one or more physicochemical parameters comprise a ratio of surface area of the surface to a volume of a sample comprising the plurality of molecules.

Embodiment 40. The computer-implemented method of Embodiment 38 or 39, wherein the one or more physicochemical parameters comprise a ratio of surface area of the surface to a concentration of the plurality of molecules in a sample.

Embodiment 41. The computer-implemented method of any one of Embodiments 38-40, wherein the one or more physicochemical parameters comprise a ratio of surface area of the surface to a mass of the plurality of molecules in a sample.

Embodiment 42. The computer-implemented method of any one of Embodiments 38-41, wherein the one or more physicochemical parameters comprise a ratio of mass of a substrate comprising the surface to a volume of a sample comprising the plurality of molecules.

Embodiment 43. The computer-implemented method of any one of Embodiments 38-42, wherein the one or more physicochemical parameters comprise a ratio of mass of a substrate comprising the surface to a concentration of the plurality of molecules in a sample.

Embodiment 44. The computer-implemented method of any one of Embodiments 38-43, wherein the one or more physicochemical parameters comprise a ratio of mass of a substrate comprising the surface to a mass of the plurality of molecules in a sample.

Embodiment 45. The computer-implemented method of Embodiment 36, wherein the one or more physicochemical parameters comprise surface chemistry.

Embodiment 46. The computer-implemented method of any one of Embodiments 38-44, wherein the one or more physicochemical parameters comprise an incubation time for the plurality of molecules to the surface.

Embodiment 47. The computer-implemented method of Embodiment 46, wherein the one or more features represent changes in quantities for the plurality of molecules with respect incubation time when the incubation time is at least 1, 15, 30, or 60 seconds.

Embodiment 48. The computer-implemented method of Embodiment 47, wherein the one or more features represent changes in quantities for the plurality of molecules with respect incubation time when the incubation time is at least 1, 15, 30, or 60 minutes.

Embodiment 49. The computer-implemented method of Embodiment 48, wherein the one or more features represent changes in quantities for the plurality of molecules with respect incubation time when the incubation time is at least 1, 2, 3, 4, 8, 12, 16, 20, or 24 hours.

Embodiment 50. The computer-implemented method of Embodiment 49, wherein the one or more features represent changes in quantities for the plurality of molecules with respect incubation time when the incubation time is at least 1, 2, 3, 4, 5, 6 or 7 days.

Embodiment 51. The computer-implemented method of any one of Embodiments 46-50, wherein the one or more features represent changes in quantities for the plurality of molecules with respect incubation time when the incubation time is at most 1, 2, 3, 4, 5, 6 or 7 days.

Embodiment 52. The computer-implemented method of Embodiment 51, wherein the one or more features represent changes in quantities for the plurality of molecules with respect incubation time when the incubation time is at most 1, 2, 3, 4, 8, 12, 16, 20, or 24 hours.

Embodiment 53. The computer-implemented method of Embodiment 52, wherein the one or more features represent changes in quantities for the plurality of molecules with respect incubation time when the incubation time is at most 1, 15, 30, or 60 minutes.

Embodiment 54. The computer-implemented method of Embodiment 53, wherein the one or more features represent changes in quantities for the plurality of molecules with respect to incubation time when the incubation time is at most 1, 15, 30, or 60 seconds.

Embodiment 55. The computer-implemented method of any one of Embodiments 1-54, wherein the input dataset comprises a first plurality of quantities measured at the first condition.

Embodiment 56. The computer-implemented method of any one of Embodiments 1-55, wherein the input dataset comprises a second plurality of quantities measured at the second condition.

Embodiment 57. The computer-implemented method of any one of Embodiments 1-56, wherein the plurality of molecules comprise at least 20, 40, 60, 80, 100, 200, 300, 400, 500, 600, 700, 800, 900, 1000, 2000, 3000, 4000, 5000, 6000, 7000, 8000, 9000, 10000, 11000, 12000, 13000, 14000, 15000, 16000, 17000, 18000, 19000, or 20000 molecules.

Embodiment 58. The computer-implemented method of any one of Embodiments 1-57, wherein the plurality of molecules comprise at most 20, 40, 60, 80, 100, 200, 300, 400, 500, 600, 700, 800, 900, 1000, 2000, 3000, 4000, 5000, 6000, 7000, 8000, 9000, 10000, 11000, 12000, 13000, 14000, 15000, 16000, 17000, 18000, 19000, or 20000 molecules.

Embodiment 59. The computer-implemented method of any one of Embodiments 1-58, wherein the one or more features comprise at least 2, 3, 4, 5, 6, 7, 8, 9, 10, 20, 30, 40, 50, 60, 70, 80, 90, or 100 features for each molecule in the plurality of molecules.

Embodiment 60. The computer-implemented method of any one of Embodiments 1-59, wherein the one or more features comprise at most 2, 3, 4, 5, 6, 7, 8, 9, 10, 20, 30, 40, 50, 60, 70, 80, 90, or 100 features for each molecule in the plurality of molecules.

Embodiment 61. The computer-implemented method of any one of Embodiments 1-60, wherein the one or more physicochemical parameters comprise at least 2, 3, 4, 5, 6, 7, 8, 9, 10, 20, 30, 40, 50, 60, 70, 80, 90, or 100 physicochemical parameters.

Embodiment 62. The computer-implemented method of any one of Embodiments 1-61, wherein the one or more physicochemical parameters comprise at most 2, 3, 4, 5, 6, 7, 8, 9, 10, 20, 30, 40, 50, 60, 70, 80, 90, or 100 physicochemical parameters.

Embodiment 63. The computer-implemented method of any one of Embodiments 1-62, wherein the output value is a normalization value for adjusting the quantities of the plurality of molecules using the first condition to predicted quantities of the molecules using the second condition.

Embodiment 64. The computer-implemented method of Embodiment 63, wherein the normalization value is the difference between a quantity and a reference quantity.

Embodiment 65. The computer-implemented method of Embodiment 63, wherein the normalization value is a ratio between a quantity and a reference quantity.

Embodiment 66. The computer-implemented method of any one of Embodiments 1-65, wherein the output value is a reference quantity.

Embodiment 67. The computer-implemented method of any one of Embodiments 1-66, further comprising predicting a predicted quantity of a molecule at the second condition using a measured quantity of the molecule at the first condition, wherein the molecule is not in the input dataset.

Embodiment 68. The computer-implemented method of Embodiment 67, wherein a magnitude of the predicted quantity is below a detection limit of the method or device used to generate the input dataset.

Embodiment 69. The computer-implemented method of Embodiment 68, wherein a first scale of the predicted quantity is different from a second scale of the method or device used to generate the machine learning model.

Embodiment 70. The computer-implemented method of Embodiment 69, wherein the second scale of the method or device comprises a deviation.

Embodiment 71. The computer-implemented method of Embodiment 70, wherein the method or device comprises MS, and wherein the deviation is based on a number of charges and flyability.

Embodiment 72. The computer-implemented method of any one of Embodiments 1-71, wherein the output value is a corrected or tuned quantity.

Embodiment 73. The computer-implemented method of any one of Embodiments 1-72, wherein the corrected quantity is a corrected MS intensity.

Embodiment 74. The computer-implemented method of any one of Embodiments 1-73, wherein the adjusting comprises at least partially optimizing a mean squared error loss function when the input dataset comprises a quantity in the quantities and a reference quantity in the reference quantities.

Embodiment 75. The computer-implemented method of any one of Embodiments 1-74, wherein the adjusting comprises at least partially optimizing a logistic loss function when the input dataset does not comprise either a quantity in the quantities or a reference quantity in the reference quantities.

Embodiment 76. The computer-implemented method of any one of Embodiments 1-75, further comprising receiving a second input dataset comprising: (a) a second set of features that represent a second set of changes in a second set of quantities for a second plurality of molecules with respect to the one or more physicochemical parameters, wherein the second set of changes are measured using at least a third condition; (b) processing, using the machine learning algorithm, the second input dataset to generate a second output value; and adjusting the one or more numerical parameters of the machine learning algorithm based on a second loss function based at least in part on the second output value.

Embodiment 77. The computer-implemented method of Embodiment 76, wherein the second plurality of molecules comprises no molecules in common with the plurality of molecules.

Embodiment 78. The computer-implemented method of Embodiment 76, wherein the second plurality of molecules comprises one or more molecules in common with the plurality of molecules.

Embodiment 79. The computer-implemented method of Embodiment 76, wherein the second plurality of molecules comprises one or more molecules not in the plurality of molecules.

Embodiment 80. The computer-implemented method of any one of Embodiments 1-79, wherein the second input dataset comprises the reference quantities.

Embodiment 81. The computer-implemented method of any one of Embodiments 1-80, wherein the input dataset comprises a plurality of differences between the quantities and the reference quantities.

Embodiment 82. The computer-implemented method of any one of Embodiments 1-81, wherein a reference quantity of a reference molecule in the reference molecules and a quantity of a molecule in the molecules have a similar change with respect to the one or more physicochemical parameters.

Embodiment 83. The computer-implemented method of any one of Embodiments 1-82, wherein the reference molecules are the same as the at least the portion of the molecules.

Embodiment 84. The computer-implemented method of any one of Embodiments 1-83, wherein the reference quantities of the reference molecules are derived from the same sample as the at least the portion of the molecules.

Embodiment 85. The computer-implemented method of any one of Embodiments 1-84, wherein the reference quantities comprise average abundance values of the molecules over a plurality of samples.

Embodiment 86. The computer-implemented method of any one of Embodiments 1-85, wherein the average abundance values are concentration values, intensities values, or relative abundance values.

Embodiment 87. The computer-implemented method of any one of Embodiments 1-86, wherein the second condition comprises a neat measurement condition.

Embodiment 88. The computer-implemented method of Embodiment 87, wherein the neat measurement condition does not comprise binding the molecule to the surface.

Embodiment 89. The computer-implemented method of any one of Embodiments 1-88, wherein the reference quantities comprise an aggregate of measurements of samples.

Embodiment 90. The computer-implemented method of any one of Embodiments 1-89, wherein the reference quantity of a reference molecule in the second input dataset is based on a reference signal of another molecule.

Embodiment 91. The computer-implemented method of any one of Embodiments 1-90, wherein the second condition comprises using liquid chromatography with a gradient length equal to or greater than 30 minutes or 2 hours.

Embodiment 92. The computer-implemented method of any one of Embodiments 1-91, wherein the second condition comprises gas phase separation.

Embodiment 93. The computer-implemented method of any one of Embodiments 1-92, wherein the second condition comprises a different ratio of surface area of the surface to a volume of a sample comprising the biomolecule compared to the first condition.

Embodiment 94. The computer-implemented method of any one of Embodiments 1-93, wherein the second condition comprises a different ratio of a surface area of the surface to a concentration of the biomolecule in a sample compared to the first condition.

Embodiment 95. The computer-implemented method of any one of Embodiments 1-94, wherein the second condition comprises a different ratio of a surface area of the surface to a mass of the biomolecule in a sample compared to the first condition.

Embodiment 96. The computer-implemented method of any one of Embodiments 1-95, wherein the second condition comprises a different ratio of a mass of a substrate comprising the surface to a volume of a sample comprising the biomolecule compared to the first condition.

Embodiment 97. The computer-implemented method of any one of Embodiments 1-96, wherein the second condition comprises a different ratio of a mass of a substrate comprising the surface to a concentration of the biomolecule in a sample compared to the first condition.

Embodiment 98. The computer-implemented method of any one of Embodiments 1-97, wherein the second condition comprises a different ratio of a mass of a substrate comprising the surface to a mass of the biomolecule in a sample compared to the first condition.

Embodiment 99. The computer-implemented method of any one of Embodiments 1-98, wherein the second condition comprises a different ratio of the biomolecule to the surface in a sample compared to the first condition.

Embodiment 100. The computer-implemented method of any one of Embodiments 1-99, wherein the second condition comprises the surface with a different surface charge compared to the first condition.

Embodiment 101. The computer-implemented method of any one of Embodiments 1-100, wherein the second condition comprises the surface with a different surface functionalization compared to the first condition.

Embodiment 102. The computer-implemented method of any one of Embodiments 1-101, wherein the second condition comprises a different incubation time for binding the biomolecule to the surface compared to the first condition.

Embodiment 103. The computer-implemented method of any one of Embodiments 1-102, wherein the plurality of molecules comprises a plurality of biomolecules.

Embodiment 104. The computer-implemented method of any one of Embodiments 1-103, wherein the plurality of molecules comprises a plurality of proteins.

Embodiment 105. The computer-implemented method of any one of Embodiments 1-104, wherein the plurality of molecules comprises a plurality of proteoforms.

Embodiment 106. The computer-implemented method of Embodiment 105, wherein the plurality of proteoforms comprises a splicing variant.

Embodiment 107. The computer-implemented method of Embodiment 105 or 106, wherein the plurality of proteoforms comprises an allelic variant.

Embodiment 108. The computer-implemented method of any one of Embodiments 105-107, wherein the plurality of proteoforms comprises a post-translational cleavage variant.

Embodiment 109. The computer-implemented method of any one of Embodiments 105-108, wherein the plurality of proteoforms comprises a phosphorylated variant.

Embodiment 110. The computer-implemented method of any one of Embodiments 1-109, wherein the plurality of molecules comprises a plurality of lipids.

Embodiment 111. The computer-implemented method of any one of Embodiments 1-110, wherein the plurality of molecules comprises a plurality of nucleic acids.

Embodiment 112. The computer-implemented method of any one of Embodiments 1-111, wherein the plurality of molecules comprises a plurality of metabolites.

Embodiment 113. The computer-implemented method of any one of Embodiments 1-112, wherein the plurality of molecules comprises a plurality of secreted molecules.

Embodiment 114. The computer-implemented method of any one of Embodiments 1-113, wherein the first condition, the second condition, or both comprises binding a molecule in the plurality of molecules to an antibody.

Embodiment 115. The computer-implemented method of Embodiment 114, wherein the first condition, the second condition, or both comprises binding the molecule to a pair of antibodies.

Embodiment 116. The computer-implemented method of Embodiment 115, wherein the pair of antibodies comprises complementary single-stranded nucleic acid sequences attached thereto, such that when the pair of antibodies bind to the molecule, the complementary nucleic acids hybridize to form a double stranded nucleic acid.

Embodiment 117. The computer-implemented method of Embodiment 116, wherein the double stranded nucleic acid is configured to form a binding complex with a polymerase and a plurality of nucleotides, nucleosides, nucleotide analogs, and/or nucleoside analogs to perform an amplification reaction to produce a detectable signal.

Embodiment 118. The computer-implemented method of any one of Embodiments 1-117, wherein the first condition, the second condition, or both comprises binding a molecule in the plurality of molecules to an aptamer.

Embodiment 119. The computer-implemented method of Embodiment 118, wherein the one or more aptamers are coupled to a surface via a cleavable linker.

Embodiment 120. The computer-implemented method of Embodiment 119, wherein the surface is a particle surface.

Embodiment 121. The computer-implemented method of Embodiment 120, wherein the cleavable linker is photocleavable.

Embodiment 122. The computer-implemented method of Embodiment 121, wherein the first condition, the second condition, or both comprises contacting the molecule and the aptamer with a macromolecular competitor configured to, in a fluid composition, reduce dissociation of a complex comprising the one or more aptamers and the molecule.

Embodiment 123. The computer-implemented method of Embodiment 122, wherein the macromolecular competitor is a polyanionic macromolecule.

Embodiment 124. The computer-implemented method of any one of Embodiments 1-123, wherein the first condition, the second condition, or both comprises protein sequencing, and the plurality of molecules comprises a plurality of proteins.

Embodiment 125. The computer-implemented method of Embodiment 124, wherein the protein sequencing comprises (i) digesting the plurality of proteins to generate a plurality of protein fragments, (ii) immobilizing the plurality of protein fragments to a semiconductor substrate, (iii) contacting the plurality of protein fragments with a plurality of labeled recognizers, wherein the plurality of labeled recognizers are configured to attach to a predetermined chemical moiety in the plurality of protein fragments at the N-terminus of the plurality of protein fragments, (iv) exciting the plurality of labeled recognizers to detect the plurality of labeled recognizers, thereby detecting the predetermined chemical moiety, (v) removing an amino acid from the N-terminus of the plurality of protein fragments, (vi) contacting the plurality of protein fragments with a second plurality of labeled recognizers, (vii) exciting the second plurality of labeled recognizers to detect a second amino acid from the N-terminus of the plurality of protein fragments, thereby performing the protein sequencing.

Embodiment 126. The computer-implemented method of any one of Embodiments 1-125, wherein the one or more features are obtained from a sample comprising the plurality of molecules.

Embodiment 127. The computer-implemented method of Embodiment 126, wherein the sample comprises at most about 1000, 100, 10, 1, 0.1, 0.01, or 0.001 nanograms of biomolecules.

Embodiment 128. The computer-implemented method of Embodiment 126 or 127, wherein the sample comprises at most about 1000, 100, 10, 1, 0.1, 0.01, or 0.001 nanograms of biomolecules per mL of the sample.

Embodiment 129. The computer-implemented method of any one of Embodiments 126-128, wherein the sample comprises biomolecules from at most about 1000, 100, 10, or 1 cell.

Embodiment 130. The computer-implemented method of any one of Embodiments 126-129, wherein the sample comprises at most about 1000, 100, 10, 1, 0.1, 0.01, or 0.001 microliters.

Embodiment 131. The computer-implemented method of any one of Embodiments 126-130, wherein the sample comprises a complex biological sample.

Embodiment 132. The computer-implemented method of any one of Embodiments 126-131, wherein the sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

Embodiment 133. The computer-implemented method of Embodiment 132, wherein the biological sample comprises plasma or serum.

Embodiment 134. The computer-implemented method of any one of Embodiments 63-133, wherein the predicted quantities of the plurality of molecules is more accurate than the quantities of the plurality of molecules by at least 1, 2, 3, 4, 5, 6, 7, 8, 9, 10, 15, 20, 25, 30, 35, 40, 45, or 50 percent.

Embodiment 135. The computer-implemented method of any one of Embodiments 63-134, wherein a coefficient of determination between the predicted quantities of the plurality of molecules and the reference quantities of the pluralities of reference molecules is at least 0.7, 0.8, 0.85, 0.9, 0.91, 0.92, 0.93, 0.94, 0.95, 0.96, 0.97, 0.98, or 0.99, when the plurality of molecules and the plurality of reference molecules are the same and the coefficient of determination is measured with a k-fold cross validation, wherein k is an integer greater than 1.

Embodiment 136. The computer-implemented method of any one of Embodiments 63-135, wherein a first coefficient of determination between the predicted quantities of the plurality of molecules and the reference quantities of the pluralities of reference molecules is greater than a second coefficient of determination between the quantities of the plurality of molecules and the reference quantities of the plurality of reference molecules by at least 0.01, 0.02, 0.03, 0.04, 0.05, 0.06, 0.07, 0.08, 0.09, 0.1, 0.2, 0.3, 0.4, or 0.5 when the plurality of molecules and the plurality of reference molecules are the same and the coefficient of determination is measured with a k-fold cross validation, wherein k is an integer greater than 1.

Embodiment 137. The computer-implemented method of any one of Embodiments 63-136, wherein a mean absolute error (MAE) between the predicted quantities of the plurality of molecules and the reference quantities of the pluralities of molecules is at most 1, 2, 3, 4, 5, 6, 7, 8, 9, 10, 15, 20, 25, or 30 percent of the standard deviation of the reference quantities when the plurality of molecules and the plurality of reference molecules are the same and the MAE is measured with a k-fold cross validation, wherein k is an integer greater than 1.

Embodiment 138. The computer-implemented method of any one of Embodiments 63-137, wherein a first mean absolute error (MAE) between the predicted quantities of the plurality of molecules and the reference quantities of the pluralities of molecules is less than a second MAE between the quantities of the plurality of molecules and the reference quantities of the plurality of molecules by at least 1, 2, 3, 4, 5, 6, 7, 8, 9, 10, 15, 20, 25, or 30 percent of the standard deviation of the reference quantities when the plurality of molecules and the plurality of reference molecules are the same and the MAE is measured with a k-fold cross validation, wherein k is an integer greater than 1.

Embodiment 139. A computer-implemented method for quantifying a molecule using a machine learning algorithm, comprising: providing an input dataset comprising one or more features representing a quantity of the molecule measured using at least a first condition; processing the input dataset, using the machine learning algorithm trained according to any one of Embodiments 1-138, to generate an adjusted quantity of the molecule at a second condition.

Embodiment 140. The computer-implemented method of Embodiment 139, wherein the input dataset comprises one or more features for a plurality of quantities of a plurality of molecules.

Embodiment 141. The computer-implemented method of Embodiment 139 or 140, wherein the plurality of quantities comprise at least 20, 40, 60, 80, 100, 200, 300, 400, 500, 600, 700, 800, 900, 1000, 2000, 3000, 4000, 5000, 6000, 7000, 8000, 9000, 10000, or 20000 quantities.

Embodiment 142. The computer-implemented method any one of Embodiments 139-141, wherein the plurality of quantities comprise at most 20, 40, 60, 80, 100, 200, 300, 400, 500, 600, 700, 800, 900, 1000, 2000, 3000, 4000, 5000, 6000, 7000, 8000, 9000, 10000, or 20000 quantities.

Embodiment 143. The computer-implemented method any one of Embodiments 139-142, wherein the plurality of molecules comprise at least 20, 40, 60, 80, 100, 200, 300, 400, 500, 600, 700, 800, 900, 1000, 2000, 3000, 4000, 5000, 6000, 7000, 8000, 9000, 10000, or 20000 molecules.

Embodiment 144. The computer-implemented method any one of Embodiments 139-143, wherein the plurality of molecules comprise at most 20, 40, 60, 80, 100, 200, 300, 400, 500, 600, 700, 800, 900, 1000, 2000, 3000, 4000, 5000, 6000, 7000, 8000, 9000, 10000, or 20000 molecules.

Embodiment 145. A computer-implemented method for training a machine learning algorithm for biomolecule quantification comprising: measuring quantities of a plurality of proteins in a sample, by: contacting the plurality of proteins with a surface to generate a plurality of adsorbed proteins; and performing mass spectrometry (MS) using the plurality of adsorbed proteins to obtain the quantities, wherein the quantities comprise a deviation or a noise introduced by the contacting in (i); repeating (a) using a set of different experimental conditions to generate a set of quantities, wherein the set of different experimental conditions are different in (i) ratios of the surface to the plurality of proteins, (ii) incubation time used for the contacting, or (iii) both; measuring reference quantities of a plurality of reference proteins in a reference sample by: performing mass spectrometry using the plurality of reference proteins, without contacting the plurality of reference proteins with the surface, to obtain the reference quantities, such that the reference quantities do not comprise the bias or the noise; processing the set of quantities to generate a first set of features that represent changes in the quantities with respect to the set of different experimental conditions; processing the set of quantities and the reference quantities to generate a second set of features that represent a quantitative difference between the quantities and the reference quantities; processing, using the machine learning algorithm, the first set of features to generate an output value; and adjusting one or more numerical parameters of the machine learning algorithm based on a loss function based at least in part on the output value and the second set of features, such that the output value accounts for the quantitative difference between the quantities and the reference quantities, thereby training the machine learning algorithm.

Embodiment 146. A computer-implemented method for using the machine learning algorithm of Embodiment 145 for molecule quantification, comprising: measuring initial quantities of a plurality of target proteins in a target sample, by: contacting the plurality of target proteins with the surface to generate a plurality of adsorbed target proteins; and performing mass spectrometry (MS) using the plurality of adsorbed target proteins to obtain the initial quantities, wherein the initial quantities comprise the bias or the noise; repeating (h) using the set of different experimental conditions to generate a set of initial quantities; processing the set of initial quantities to generate a third set of features that represent changes in the initial quantities with respect to the set of different experimental conditions; processing, using the machine learning algorithm, the third set of features to generate an output value; and using the output value to adjust the initial quantities to generate adjusted quantities, wherein the adjusted quantities comprise less of the bias or the noise.

Embodiment 147. A computer program product comprising a computer-readable medium having computer-executable code encoded therein, the computer-executable code adapted to be executed to implement any one of the methods of Embodiments 1-146.

Embodiment 148. A non-transitory computer-readable storage media encoded with a computer program including instructions executable by one or more processors to implement any one of the methods of Embodiments 1-146.

Embodiment 149. A computer-implemented system comprising: a digital processing device comprising: at least one processor, an operating system configured to perform executable instructions, a memory, and a computer program including instructions executable by the digital processing device to perform any one of the methods of Embodiments 1-146.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A computer-implemented method for training a machine learning algorithm for molecule quantification comprising:
a. providing an input dataset comprising one or more features that represent changes in quantities for a plurality of molecules with respect to one or more physicochemical parameters, wherein the changes are measured using at least a first condition;
b. processing, using the machine learning algorithm, the input dataset to generate an output value; and
c. adjusting one or more numerical parameters of the machine learning algorithm based on a loss function based at least in part on the output value, such that the output value accounts for a difference between (i) the quantities for at least a portion of the plurality of molecules and (ii) reference quantities for a plurality of reference molecules, wherein the reference quantities are measured using at least a second condition.

2. The computer-implemented method of claim 1, wherein the first condition comprises binding the plurality of molecules to a surface.

3. The computer-implemented method of claim 2, wherein the surface comprises a particle surface.

4. The computer-implemented method of claim 1, wherein the quantities and the reference quantities comprise measured intensities.

5. The computer-implemented method of claim 4, wherein the measured intensities comprise mass spectrometry (MS) intensities.

6. The method of claim 1, wherein the plurality of molecules comprises a plurality of proteins.

7. The method of claim 6, wherein the input dataset comprises measured intensities of a plurality of peptides, wherein the plurality of peptides is derived from the plurality of proteins.

8. The computer-implemented method of claim 5, wherein the MS intensities comprise small molecule intensities.

9. The computer-implemented method of claim 2, wherein the one or more physicochemical parameters comprise a ratio of surface area of the surface to a volume of a sample comprising the plurality of molecules.

10. The computer-implemented method of claim 1, wherein the input dataset comprises a first plurality of quantities measured at the first condition and a second plurality of quantities measured at the second condition.

11. The computer-implemented method of claim 1, wherein the output value is a normalization value for adjusting the quantities of the plurality of molecules using the first condition to predicted quantities of the plurality of molecules using the second condition.

12. The computer-implemented method of claim 1, further comprising predicting a predicted quantity of a molecule at the second condition using a measured quantity of the molecule at the first condition, wherein the molecule is not in the input dataset.

13. The computer-implemented method of claim 12, wherein a magnitude of the predicted quantity is below a detection limit of the method or device used to generate the input dataset.

14. The computer-implemented method of claim 1, wherein the adjusting comprises at least partially optimizing a mean squared error loss function when the input dataset comprises a quantity in the quantities and a reference quantity in the reference quantities.

15. The computer-implemented method of claim 1, wherein the adjusting comprises at least partially optimizing a logistic loss function when the input dataset does not comprise either a quantity in the quantities or a reference quantity in the reference quantities.

16. The computer-implemented method of claim 1, further comprising receiving a second input dataset comprising: (a) a second set of features that represent a second set of changes in a second set of quantities for a second plurality of molecules with respect to the one or more physicochemical parameters, wherein the second set of changes are measured using at least a third condition; (b) processing, using the machine learning algorithm, the second input dataset to generate a second output value; and (c) adjusting the one or more numerical parameters of the machine learning algorithm based on a second loss function based at least in part on the second output value.

17. The computer-implemented method of claim 16, wherein the second plurality of molecules comprises one or more molecules not in the plurality of molecules.

18. The computer-implemented method of claim 16, wherein the second input dataset comprises the reference quantities or a plurality of differences between the quantities and the reference quantities.

19. The computer-implemented method of claim 18, wherein the reference quantity of a reference molecule in the second input dataset is based on a reference signal of another molecule.

20. The computer-implemented method of claim 1, wherein the second condition comprises a neat measurement condition.

21. The computer-implemented method of claim 1, wherein the one or more features are obtained from a sample comprising the plurality of molecules, wherein the sample comprises plasma or serum.

22. A computer-implemented method for quantifying a molecule using a machine learning algorithm, comprising:
(a) providing an input dataset comprising one or more features representing a quantity of the molecule measured using at least a first condition;
(b) processing the input dataset, using the machine learning algorithm trained according to claim 1, to generate an adjusted quantity of the molecule at a second condition.

23. A computer-implemented method for training a machine learning algorithm for biomolecule quantification comprising:
(a) measuring quantities of a plurality of proteins in a sample, by:
(i) contacting the plurality of proteins with a surface to generate a plurality of adsorbed proteins; and
(ii) performing mass spectrometry (MS) using the plurality of adsorbed proteins to obtain the quantities, wherein the quantities comprise a deviation or a noise introduced by the contacting in (i);
(b) repeating (a) using a set of different experimental conditions to generate a set of quantities, wherein the set of different experimental conditions are different in (i) ratios of the surface to the plurality of proteins, (ii) incubation time used for the contacting, or (iii) both;
(c) measuring reference quantities of a plurality of reference proteins in a reference sample by:
(i) performing mass spectrometry using the plurality of reference proteins, without contacting the plurality of reference proteins with the surface, to obtain the reference quantities, such that the reference quantities do not comprise the bias or the noise;
(d) processing the set of quantities to generate a first set of features that represent changes in the quantities with respect to the set of different experimental conditions;
(e) processing the set of quantities and the reference quantities to generate a second set of features that represent a quantitative difference between the quantities and the reference quantities;
(f) processing, using the machine learning algorithm, the first set of features to generate an output value; and
(g) adjusting one or more numerical parameters of the machine learning algorithm based on a loss function based at least in part on the output value and the second set of features, such that the output value accounts for the quantitative difference between the quantities and the reference quantities, thereby training the machine learning algorithm.

24. A computer-implemented method for using the machine learning algorithm of claim 23 for molecule quantification, comprising:
(h) measuring initial quantities of a plurality of target proteins in a target sample, by:
i. contacting the plurality of target proteins with the surface to generate a plurality of adsorbed target proteins; and
ii. performing mass spectrometry (MS) using the plurality of adsorbed target proteins to obtain the initial quantities, wherein the initial quantities comprise the bias or the noise;
(i) repeating (h) using the set of different experimental conditions to generate a set of initial quantities;
(j) processing the set of initial quantities to generate a third set of features that represent changes in the initial quantities with respect to the set of different experimental conditions;
(k) processing, using the machine learning algorithm, the third set of features to generate an output value; and
(l) using the output value to adjust the initial quantities to generate adjusted quantities, wherein the adjusted quantities comprise less of the bias or the noise.
